SEQUENCE LISTING (75 sequences)

SEQ ID NO: 1 - DNA, 101 bp, Capsicum annuum
  variation 51: note = Polymorphism: Susceptible=G;Resistant=A
  source 1..101: mol_type = unassigned DNA
aagatactctgcattgggtccgggctagaagaataagcacgaataaccgcrttccaagaaaatagatttggttgaggaatttgatcgaacacttggtgggc

SEQ ID NO: 2 - DNA, 46 bp, synthetic construct
  misc_feature 1..46: note = sequence of a forward primer for detecting the susceptible alleleof PE-0014628
  source 1..46: mol_type = other DNA
gaaggtcggagtcaacggattagaagaataagcacgaataaccgcg

SEQ ID NO: 3 - DNA, 46 bp, synthetic construct
  misc_feature 1..46: note = sequence of a forward primer for detecting the resistant alleleof PE-0014628
  source 1..46: mol_type = other DNA
gaaggtgaccaagttcatgctagaagaataagcacgaataaccgca

SEQ ID NO: 4 - DNA, 30 bp, synthetic construct
  misc_feature 1..30: note = sequence of a common reverse primer for PE-0014628
  source 1..30: mol_type = other DNA
aattcctcaaccaaatctattttcttggaa

SEQ ID NO: 5 - DNA, 301 bp, Capsicum annuum
  variation 151: note = Polymorphism: Susceptible=T;Resistant=C
  source 1..301: mol_type = unassigned DNA
attttctaggttactggatttaactcaggagaaaatggatttgtttttgcaatgcatttttccgggggtgcctagtttataaagacctaagatgtttcagtcaactgcttaattaggacctttgactgttgtggttatgactgggcccctygccaagatggttttggacaacagagctcttaccacacttgctgctattttgatctacttgatcttgagttgcgctgtttgctgtgctgttcaaagcgacattgactgtctgagatctataaaagattcattagaagatccaaataatact

SEQ ID NO: 6 - DNA, 42 bp, synthetic construct
  misc_feature 1..42: note = sequence of a forward primer for detecting the susceptible alleleof PE-0056542
  source 1..42: mol_type = other DNA
gaaggtcggagtcaacggattgtggttatgactgggcccctt

SEQ ID NO: 7 - DNA, 42 bp, synthetic construct
  misc_feature 1..42: note = sequence of a forward primer for detecting the resistant alleleof PE-0056542
  source 1..42: mol_type = other DNA
gaaggtgaccaagttcatgctgtggttatgactgggcccctc

SEQ ID NO: 8 - DNA, 28 bp, synthetic construct
  misc_feature 1..28: note = sequence of a common reverse primer for PE-0056542
  source 1..28: mol_type = other DNA
taagagctctgttgtccaaaaccatctt

SEQ ID NO: 9 - DNA, 301 bp, Capsicum annuum
  variation 151: note = Polymorphism: Susceptible=G;Resistant=A
  source 1..301: mol_type = unassigned DNA
agatggttttggacaacagagctcttaccacacttgctgctattttgatctacttgatcttgagttgcgctgtttgctgtgctgttcaaagcgacattgactgtctgagatctataaaagattcattagaagatccaaataatactttgarctatacatggaagttcgataatctgacggaaggtttcatttgcaagtttgctgggattcagtgctggcatcctgatgaaaataaggttctgagtatcagtcttcctgacatgggtctcaagggcaggtttccacttggtcttcggaattg

SEQ ID NO: 10 - DNA, 49 bp, synthetic construct
  misc_feature 1..49: note = sequence of a forward primer for detecting the susceptible alleleof PE-0056543
  source 1..49: mol_type = other DNA
gaaggtcggagtcaacggattcattagaagatccaaataatactttgag

SEQ ID NO: 11 - DNA, 54 bp, synthetic construct
  misc_feature 1..54: note = sequence of a forward primer for detecting the resistant alleleof PE-0056543
  source 1..54: mol_type = other DNA
gaaggtgaccaagttcatgctagattcattagaagatccaaataatactttgaa

SEQ ID NO: 12 - DNA, 29 bp, synthetic construct
  misc_feature 1..29: note = sequence of a common reverse primer for PE-0056543
  source 1..29: mol_type = other DNA
atgaaaccttccgtcagattatcgaactt

SEQ ID NO: 13 - DNA, 301 bp, Capsicum annuum
  variation 151: note = Polymorphism: Susceptible=G;Resistant=T
  source 1..301: mol_type = unassigned DNA
ctgttcctgcagagagctatgccaataatcccggactgtgtggagaaccgttagatccttgccagggtacttctaagaaaccccctaccgcaatcattgtcggtgcagctgttggtggggtaagtttaggtgctgtatttttggccatcgktatgttcttctatatgagaaggatatccaggaagaggaagaaggatgatgatcctgaagggaataaatgggcgaagagtattaagggtaaaaaggcaatccaggtaagaattttaacttctctgaatcattaaaaactgtctcgttactg

SEQ ID NO: 14 - DNA, 44 bp, synthetic construct
  misc_feature 1..44: note = sequence of a forward primer for detecting the susceptible alleleof PE-0056544
  source 1..44: mol_type = other DNA
gaaggtgaccaagttcatgctggtgctgtatttttggccatcgg

SEQ ID NO: 15 - DNA, 45 bp, synthetic construct
  misc_feature 1..45: note = sequence of a forward primer for detecting the resistant alleleof PE-0056544
  source 1..45: mol_type = other DNA
gaaggtcggagtcaacggattaggtgctgtatttttggccatcgt

SEQ ID NO: 16 - DNA, 29 bp, synthetic construct
  misc_feature 1..29: note = sequence of a common reverse primer for PE-0056544
  source 1..29: mol_type = other DNA
ttcctcttcctggatatccttctcatata

SEQ ID NO: 17 - DNA, 301 bp, Capsicum annuum
  variation 151: note = Polymorphism: Susceptible=T;Resistant=C
  source 1..301: mol_type = unassigned DNA
tcttttcatagaagcatttaactaatgaattttcacctgctccgctgctcgtgtgtgatttttctcttgttcaagttttcttaattgacttaaggttccttattttcagctttcaatgtttgagaagtctacttcaaaaatgagatttagygatcttatgaaggccactgacagcttcaacaaaaacaatattatcgggtcaggaagaaccgggactttttacaaagcagtacttgatgatggcacttcacttatggttaagaggttgcagaatactcagcactcagagaaagagtttatg

SEQ ID NO: 18 - DNA, 45 bp, synthetic construct
  misc_feature 1..45: note = sequence of a forward primer for detecting the susceptible alleleof PE-0056545
  source 1..45: mol_type = other DNA
gaaggtcggagtcaacggattctgtcagtggccttcataagatca

SEQ ID NO: 19 - DNA, 43 bp, synthetic construct
  misc_feature 1..43: note = sequence of a forward primer for detecting the resistant alleleof PE-0056545
  source 1..43: mol_type = other DNA
gaaggtgaccaagttcatgctgtcagtggccttcataagatcg

SEQ ID NO: 20 - DNA, 30 bp, synthetic construct
  misc_feature 1..30: note = sequence of a common reverse primer for PE-0056545
  source 1..30: mol_type = other DNA
gtttgagaagtctacttcaaaaatgagatt

SEQ ID NO: 21 - DNA, 301 bp, Capsicum annuum
  variation 151: note = Polymorphism: Susceptible=A ; Resistant=C
  source 1..301: mol_type = unassigned DNA
actcagcactcagagaaagagtttatgtccgagatggctacattgggaaatgtaaagcatcgtaatctggttcctcttttaggtttctgcattgccaaaaaagaaaggctgttggtctacaaagacatgccaaatggaaccttgcatgatmggttacattcggtgagtgaaggcgaaaaaactctagagtggcctataagattaaaaataggcattggagcagccaaaggatttgcatggcttcaccacaactgcaatcctcgtatcattcacagaaatattagttctaaatgcatcttgc

SEQ ID NO: 22 - DNA, 44 bp, synthetic construct
  misc_feature 1..44: note = sequence of a forward primer for detecting the susceptible alleleof PE-0056546
  source 1..44: mol_type = other DNA
gaaggtgaccaagttcatgctccttcactcaccgaatgtaacct

SEQ ID NO: 23 - DNA, 44 bp, synthetic construct
  misc_feature 1..44: note = sequence of a forward primer for detecting the resistant alleleof PE-0056546
  source 1..44: mol_type = other DNA
gaaggtcggagtcaacggattccttcactcaccgaatgtaaccg

SEQ ID NO: 24 - DNA, 28 bp, synthetic construct
  misc_feature 1..28: note = sequence of a common reverse primer for PE-0056546
  source 1..28: mol_type = other DNA
ttggtctacaaagacatgccaaatggaa

SEQ ID NO: 25 - DNA, 301 bp, Capsicum annuum
  variation 151: note = Polymorphism: Susceptible=A ; Resistant=C
  source 1..301: mol_type = unassigned DNA
gctactccaaaaggtgatgtgtacagtttcggtgttgtacttctcgagttagttactggtgagaaaccgacctctgtcaccaaagctcccgagacctttaaggggaatttggtggaatggatcacacaactctccggtgaatctaagcttmaagatgcgattgaccattcattgtctggtaaaggttatgacaatgagatcttccaggtccttaaagttgcttgtcgatgtgtgttgtctcctgttcctaaggagaggccgacaatgtttgaagtttaccagcttctgagagccatcggag

SEQ ID NO: 26 - DNA, 46 bp, synthetic construct
  misc_feature 1..46: note = sequence of a forward primer for detecting the susceptible alleleof PE-0056547
  source 1..46: mol_type = other DNA
gaaggtgaccaagttcatgctcaactctccggtgaatctaagctta

SEQ ID NO: 27 - DNA, 45 bp, synthetic construct
  misc_feature 1..45: note = sequence of a forward primer for detecting the resistant alleleof PE-0056547
  source 1..45: mol_type = other DNA
gaaggtcggagtcaacggattaactctccggtgaatctaagcttc

SEQ ID NO: 28 - DNA, 30 bp, synthetic construct
  misc_feature 1..30: note = sequence of a common reverse primer for PE-0056547
  source 1..30: mol_type = other DNA
cattgtcataacctttaccagacaatgaat

SEQ ID NO: 29 - DNA, 301 bp, Capsicum annuum
  variation 151: note = Polymorphism: Susceptible= T; Resistant=A
  source 1..301: mol_type = unassigned DNA
taactgatggtttcagttgagctgcaaaatggaacatcaatatcagtagcttctcgtttcaaccatttacttcctttgctctttagtcacttgcactttagagaagaatgacgtgattcccgatattttatacttagctattctagcatawgggaaacaaaacagatgaatcatgaaactcttcggtagtaaaaaaaattatgtgccaaaatgttggatgtatctttttatggacgatgatccataccatgttcggattcaggatccacttttacgattgcatcctcccagctcttccatt

SEQ ID NO: 30 - DNA, 53 bp, synthetic construct
  misc_feature 1..53: note = sequence of a forward primer for detecting the susceptible alleleof PE-0056549
  source 1..53: mol_type = other DNA
gaaggtcggagtcaacggattccgatattttatacttagctattctagcatat

SEQ ID NO: 31 - DNA, 53 bp, synthetic construct
  misc_feature 1..53: note = sequence of a forward primer for detecting the resistant alleleof PE-0056549
  source 1..53: mol_type = other DNA
gaaggtgaccaagttcatgctccgatattttatacttagctattctagcataa

SEQ ID NO: 32 - DNA, 30 bp, synthetic construct
  misc_feature 1..30: note = sequence of a common reverse primer for PE-0056549
  source 1..30: mol_type = other DNA
gagtttcatgattcatctgttttgtttccc

SEQ ID NO: 33 - DNA, 301 bp, Capsicum annuum
  variation 151: note = Polymorphism: Susceptible=C Resistant=T
  source 1..301: mol_type = unassigned DNA
tttgattttgtttaactcatttgcatgaaatgatatgtttatatgtttgtctagaaatatcacaactattacatttataggtaatatttgagttgcacttatggtatagtgcatttttatttctatttatgtgacattattttttagatayatgtcgaaataatgctggaaaattaatgttcaactctttgaatacatgaaagaaccgaaacttctaccctatttttaagggatggacgcgtgaattagacctaattaagaaatatttatcttacactttttgttttatcgagaagctttt

SEQ ID NO: 34 - DNA, 51 bp, synthetic construct
  misc_feature 1..51: note = sequence of a forward primer for detecting the susceptible alleleof PE-0056550
  source 1..51: mol_type = other DNA
gaaggtgaccaagttcatgctacattaattttccagcattatttcgacatg

SEQ ID NO: 35 - DNA, 53 bp, synthetic construct
  misc_feature 1..53: note = sequence of a forward primer for detecting the resistant alleleof PE-0056550
  source 1..53: mol_type = other DNA
gaaggtcggagtcaacggattgaacattaattttccagcattatttcgacata

SEQ ID NO: 36 - DNA, 30 bp, synthetic construct
  misc_feature 1..30: note = sequence of a common reverse primer for PE-0056550
  source 1..30: mol_type = other DNA
taggtaatatttgagttgcacttatggtat

SEQ ID NO: 37 - DNA, 101 bp, Capsicum annuum
  variation 51: note = Polymorphism: Susceptible=G;Resistant=C
  source 1..101: mol_type = unassigned DNA
ctccaaaaccacagctactaatttgttagatccaaaacttaaaagcttatstttagtcagtcaattaaaaatcccaacaatttaaaagctaagaagtgaag

SEQ ID NO: 38 - DNA, 49 bp, synthetic construct
  misc_feature 1..49: note = sequence of a forward primer for detecting the susceptible alleleof PE-0021476
  source 1..49: mol_type = other DNA
gaaggtcggagtcaacggattgttgggatttttaattgactgactaaac

SEQ ID NO: 39 - DNA, 50 bp, synthetic construct
  misc_feature 1..50: note = sequence of a forward primer for detecting the resistant alleleof PE-0021476
  source 1..50: mol_type = other DNA
gaaggtgaccaagttcatgcttgttgggatttttaattgactgactaaag

SEQ ID NO: 40 - DNA, 27 bp, synthetic construct
  misc_feature 1..27: note = sequence of a common reverse primer for PE-0021476
  source 1..27: mol_type = other DNA
ctccaaaaccacagctactaatttgtt

SEQ ID NO: 41 - DNA, 1557 bp, Capsicum annuum
  source 1..1557: mol_type = unassigned DNA
atgggtggaggtggagaagatgaagggtcattggaatttacaccaacatgggttgttgctggtgtatgcactgtcattgttgcaatatctctaactcttgaacgcctcctccattatactggaaagcatttgaagaagaagaagcaaaagcatctctatgaagcactacagaaagttaaagaagagttgatgctcttggggttcatttcccttttgttaacggtatttcaaagtcgaattgtcaaaatctgtgtgcctcatcgtgtcgtaacacacttacttccatgctcattatcattggagggttcttcacattcaggttcatcgttggggggttctccatctccatctccacctgaagtgcctcgtcctagcaacaattctgttcactatcatcctggtcctcaccatcaacggcatttgcttgcagcagaagagactcctaagggttactgcagtcataaacacaaggtcccgttactatctcttgaggcgttgcatcaccttcacatttttatctttgtcctagctattgtgcatgtgactttttctgttctgactattgtatttggaggagtaaagatacgtcaatggaagagctgggaggatgcaatcgtaaaagtggatcctgaatccgaacatgctcaactgaaaccatcagttactcatgtccatgaacatgatttcatcaggaatcggtttcagggtatcggtaaacaatcactcgttatgggttgggcgcattctttcttcaagcaattttatgcttctgtaaacgagtcagactacagagcgcttcgtttggggttcattatgacacattgcaggggaaatccaagattcaatttctataggtacatggtacgtgctctggaagacgattttacgacagttgtcggtatcaggcaagttggcatacgtatttctggattgctttcgttccttttatatacaaatcttatgcaggaggaagctaacttgatttctgtgtcctttcgttcttcttttcttttcgtgttttttttccagcttctacttgcagttggaacaaagttggagcatgtgattatacagttagctcatgaaattgctgagagacatgtagcagtagaaggtgaattgattgtaacaccatccgatgatcacttttggttcgatcgccctcaaattatcctcttcttgatacattttattctattccaaaatgcttttgaaatagcatttttcttctggattttgttccaatatggctttttctcctgcattatgggaaaatacgtgtttgttattcctcggcttgtcataggggttatcattcaagttttatgcagctatagcacattgccactttatgctctagttacacagatggggagtcattataagaagtcaatgttcgataaacatgttcaagcttgcctcctggattgggctgataaggcaaaaaagaagaagggacttaagattgctagccctgtccattcaattgacggttctgttcaaggatcaccaacgtccgtaagtgggcgcaaagactaccctcaaaatagggtttag

SEQ ID NO: 42 - DNA, 1866 bp, Capsicum annuum
  source 1..1866: mol_type = unassigned DNA
atgactgggccccttgccaagatggttttggacaacagagctcttaccacacttgctgctattttgatctacttgatcttgagttgcgctgtttgctgtgctgttcaaagcgacattgactgtctgagatctataaaagattcattagaagatccaaataatactttgagctatacatggaagttcgataatctgacggaaggtttcatttgcaagtttgctgggattcagtgctggcatcctgatgaaaataaggttctgagtatcagtcttcctgacatgggtctcaagggcaggtttccacttggtcttcggaattgcacctctatgacatcattagatctttcgagcaacaatctccatggaagcattccatctgatatctccaaatatataggtttcgtggtaaccctggatctctcatctaaccaactatcaggggaaatcccagttaatcttgcaaactgctcttttttgaataaccttaaacttgaaaacaaccagtttacgggtcaaatccctccagaaattggcctgcttggtcgcctcaaaacttttaatgtagcaaacaatcgattgactggaccagttccacatttcataaatgcaactgttcctgcagagagctatgccaataatcccggactgtgtggagaaccgttagatccttgccagggtacttctaagaaaccccctaccgcaatcattgtcggtgcagctgttggtggggtaagtttaggtgctgtatttttggccatcggtatgttcttctatatgagaaggatatccaggaagaggaagaaggatgatgatcctgaagggaataaatgggcgaagagtattaagggtaaaaaggcaatccagctttcaatgtttgagaagtctacttcaaaaatgagatttagtgatcttatgaaggccactgacagcttcaacaaaaacaatattatcgggtcaggaagaaccgggactttttacaaagcagtacttgatgatggcacttcacttatggttaagaggttgcagaatactcagcactcagagaaagagtttatgtccgagatggctacattgggaaatgtaaagcatcgtaatctggttcctcttttaggtttctgcattgccaaaaaagaaaggctgttggtctacaaagacatgccaaatggaaccttgcatgataggttacattcggtgagtgaaggcgaaaaaactctagagtggcctataagattaaaaataggcattggagcagccaaaggatttgcatggcttcaccacaactgcaatcctcgtatcattcacagaaatattagttctaaatgcatcttgctggatgtggaatttgaacctaggatatcagattttgggcttgccaggctaatgaatccaattgacactcatttgagtacctttgtcaatggagaattcggggacttcggttatgttgctcctgagtacacacgaactctcatggctactccaaaaggtgatgtgtacagtttcggtgttgtacttctcgagttagttactggtgagaaaccgacctctgtcaccaaagctcccgagacctttaaggggaatttggtggaatggatcacacaactctccggtgaatctaagcttaaagatgcgattgaccattcattgtctggtaaaggttatgacaatgagatcttccaggtccttaaagttgcttgtcgatgtgtgttgtctcctgttcctaaggagaggccgacaatgtttgaagtttaccagcttctgagagccatcggagagcgatatcatttcacaactgatgatgacattttgatgctttcagagagtgatggcggattccaaatggacgagctcattgttgctcaataa

SEQ ID NO: 43 - DNA, 23 bp, synthetic construct
  misc_feature 1..23: note = sequence of a forward primer for amplifying the susceptibleallele of the RZ marker
  source 1..23: mol_type = other DNA
catgtgagcgactcctctgacaa

SEQ ID NO: 44 - DNA, 22 bp, synthetic construct
  misc_feature 1..22: note = sequence of a forward primer for amplifying the resistant alleleof the RZ marker
  source 1..22: mol_type = other DNA
atgtgagcgactcctctgacag

SEQ ID NO: 45 - DNA, 29 bp, synthetic construct
  misc_feature 1..29: note = sequence of a common reverse primer for amplifying the resistantor susceptible allele of the RZ marker
  source 1..29: mol_type = other DNA
cgaggagaatttcctgctaaaattgagtt

SEQ ID NO: 46 - DNA, 101 bp, Capsicum annuum
  variation 51: note = Polymorphism: LT17=C; PBC167=G
  source 1..101: mol_type = unassigned DNA
ttgaaaccctccttcctttcatacattgaaagaaatggtggatggagactsagcaaatcattggtcacatcatacaacctcaagagcatactagaagctga

SEQ ID NO: 47 - DNA, 101 bp, Capsicum annuum
  variation 51: note = Polymorphism: LT17=A; PBC167=G
  source 1..101: mol_type = unassigned DNA
agttgccttggtaccaatgaaattgactcttcttttccttccaagtccacrgctccaggacctgcattcagatccagaccttgccggcttcggcttctgtt

SEQ ID NO: 48 - DNA, 101 bp, Capsicum annuum
  variation 51: note = Polymorphism: LT17=C; PBC167=A
  source 1..101: mol_type = unassigned DNA
ggtggtagaatcctccctcgtcgctctgacggcacgctccgttacatcggmggttttactagagtcctctccgttgatcggtctatttcttattcagagtt

SEQ ID NO: 49 - DNA, 101 bp, Capsicum annuum
  variation 51: note = Polymorphism: LT17=T; PBC167=G
  source 1..101: mol_type = unassigned DNA
tggttctaatgtggtgacaaaggttccatcaagtgaatgttatggtgatgktggtgtaagtgctaacgtgcagaagagtaaagcaaatgaagcaaacgggg

SEQ ID NO: 50 - DNA, 101 bp, Capsicum annuum
  variation 51: note = Polymorphism: LT17=T; PBC167=C
  source 1..101: mol_type = unassigned DNA
gagatagaaaatgaaggtgaacaaaactttggatctccagctgcaccagtycaggaacgaccaattcagagatcatgggttcctccccagcctcctccagt

SEQ ID NO: 51 - DNA, 101 bp, Capsicum annuum
  variation 51: note = Polymorphism: LT17=C; PBC167=A
  source 1..101: mol_type = unassigned DNA
aaaagaatccgccctaactgctttctgctcttcttcttcacattgacttcmtcggcatcatctagaaccaaattcatgtactcatcaaaaccaataatacg

SEQ ID NO: 52 - DNA, 101 bp, Capsicum annuum
  variation 51: note = Polymorphism: LT17=G; PBC167=A
  source 1..101: mol_type = unassigned DNA
ataatgatgtcaccagtctgaactaccgaagacagttctttgcttcaatcratatgctacaagaacaagctgagaaattacttgaagagatgaagcctaag

SEQ ID NO: 53 - DNA, 121 bp, Capsicum annuum
  variation 61: note = Polymorphism: LT17=G; PBC167=A
  source 1..121: mol_type = unassigned DNA
gtccctctttcaagagttaaagaagtggtgtgtgggatttgctttgactactacccgcctratgacataatgtctgttggttgtggtcatcctttttgtactatttgctggaaagcttaca

SEQ ID NO: 54 - DNA, 208 bp, Capsicum annuum
  variation 59: note = Polymorphism: LT17=T; PBC167=G
  source 1..208: mol_type = unassigned DNA
aktaaagagaacacctgtattcaccagtactactacccagaatgggtcaatttacacckatcttggacaaaatgatgacatccatccctcctatcttattgaagcatgctacattcagtcttgcactttgacagataagtgtttcaccacatatccaataagatgcacaccatataacattgatgctggcaagagtatgcacctgcag

SEQ ID NO: 55 - DNA, 121 bp, Capsicum annuum
  variation 61: note = Polymorphism: LT17=C; PBC167=A
  source 1..121: mol_type = unassigned DNA
ggaggcaacaacacaaagtatcccactaccccccactaagatggggatagtgaaacaaggmatagtgtccattcttggatcagactctgacaaaagaaacaaggctgctgcagctgtttca

SEQ ID NO: 56 - DNA, 121 bp, Capsicum annuum
  variation 61: note = Polymorphism: LT17=T; PBC167=C
  source 1..121: mol_type = unassigned DNA
gaacgcttatgggattgttccaggggtaaaacattacgcttgcttagttgatatacttggycgtgcaggtgaactggaagaagctgaagaactgataaataatatgccagtaacccctggt

SEQ ID NO: 57 - DNA, 121 bp, Capsicum annuum
  variation 61: note = Polymorphism: LT17=A; PBC167=G
  source 1..121: mol_type = unassigned DNA
cggatattggagaagatgaaggctcctaaaaagccagatgatgtgcctgagatcacgcctragcagctcttggaagctgatggtttcatctttggtttcccttcgcgttttggtgtgatgg

SEQ ID NO: 58 - DNA, 121 bp, Capsicum annuum
  variation 61: note = Polymorphism: LT17=A; PBC167=G
  source 1..121: mol_type = unassigned DNA
tcggttgcttgcaggttattgcagtattggtgagaccagacaaggaatcaaaaataagaaratactggaattggtaccattacacagcaggaagaattttgatattattggcatcaataaa

SEQ ID NO: 59 - DNA, 101 bp, Capsicum annuum
  variation 51: note = Polymorphism: LT17=G; PBC167=C
  source 1..101: mol_type = unassigned DNA
atgtcttacaaacctccattgacaacaaagattacattgcgggcaaactcstccacgcccatatactccgacttggcctttcctctcatacactcctccta

SEQ ID NO: 60 - DNA, 121 bp, Capsicum annuum
  variation 61: note = Polymorphism: LT17=A; PBC167=T
  source 1..121: mol_type = unassigned DNA
tatacaagasaatgttggaaggggaatgggaaaatggaatatcagctggcacaattgtccwagcagcagtagctttattgcttgatggaacatcattgtatagattcatgaaggcagaggg

SEQ ID NO: 61 - DNA, 121 bp, Capsicum annuum
  variation 61: note = Polymorphism: LT17=A; PBC167=C
  source 1..121: mol_type = unassigned DNA
tgtgtagttgttggcattaaaatttgcttctagcctttcagaaaagacattacgttgtaamcttgtagtgtgtatttaattctcttgacatgaaatgaagtcgagtggacacaacaaaaaa

SEQ ID NO: 62 - DNA, 121 bp, Capsicum annuum
  variation 61: note = Polymorphism: LT17=A; PBC167=G
  source 1..121: mol_type = unassigned DNA
ctaggcctagttgatgggactacacaatggtatgcatgcaaactaagcaaaagatggggcrcgatggaggaaaaagatctgcataccagcaacttgttgggcatatattttagtcatgcta

SEQ ID NO: 63 - DNA, 101 bp, Capsicum annuum
  variation 51: note = Polymorphism: LT17=T; PBC167=C
  source 1..101: mol_type = unassigned DNA
gaatgtttttgcgttttgtggtaccacactctcttgatcatgctcaaatgyttcaaaagcatacatgcatttaaagaatccttcagcagcaaatccagaag

SEQ ID NO: 64 - DNA, 101 bp, Capsicum annuum
  variation 51: note = Polymorphism: LT17=A; PBC167=C
  source 1..101: mol_type = unassigned DNA
tgaatccaaacagatttgggacactgactgggactgatgaactcataagtmttgcctttctttgcctccttttgtttcgttttcttcctttctgttggtta

SEQ ID NO: 65 - DNA, 101 bp, Capsicum annuum
  variation 51: note = Polymorphism: LT17=A; PBC167=G
  source 1..101: mol_type = unassigned DNA
tgatcataatatcaaatctgatgttcaacaatacaatatgcacaagaggaraaaaactggttcataaatggtgaatcaagaaggcaatacgacgagccttt

SEQ ID NO: 66 - DNA, 121 bp, Capsicum annuum
  variation 61: note = Polymorphism: LT17=C; PBC167=T
  source 1..121: mol_type = unassigned DNA
ctcccgtcatctgttaaatgcttcattgcttcagacattattaagtgctagaacttcaggygttcgttcaccttgttatattgtacatgacaatgttgatgcatgtggtagcatttgtttg

SEQ ID NO: 67 - DNA, 101 bp, Capsicum annuum
  variation 51: note = Polymorphism: LT17=C; PBC167=G
  source 1..101: mol_type = unassigned DNA
tactccttcaggtggatctaagggtgaaccaagtggaaattcaggatacasagtcaagctgccatattctctcttgtttacccttgtagttgcagcatcat

SEQ ID NO: 68 - DNA, 210 bp, Capsicum annuum
  variation 89: note = Polymorphism: LT17=A; PBC167=G
  source 1..210: mol_type = unassigned DNA
ttgaattagctggtaatgcaagcaaggatcttaaagtgaagagaatcacccccaggcacttgcaactggccattcgcggagacgaggarcttgacacacttatcaaagggaccattgctggtggtggtgtcatccctcacattcacaagtccctaataaacaaaacctccaaggagtgaatttctccttttttatcttagatgtatcatg

SEQ ID NO: 69 - DNA, 120 bp, Capsicum annuum
  variation 60: note = Polymorphism: LT17=T; PBC167=C
  source 1..120: mol_type = unassigned DNA
ttaaatgttcaacaagagtccattctacagcacaatgagcaagtttggtaaagggccaaygcgatgagcctgtttgctttatatacttacggaggaaaactcaaacatgtgataagtaca

SEQ ID NO: 70 - DNA, 101 bp, Capsicum annuum
  variation 51: note = Polymorphism: LT17=C; PBC167=T
  source 1..101: mol_type = unassigned DNA
gttgcttagtctaagagagacaatagctctttttaagcttaattcaccacyattatctacttgcacttgaaaagtccaaagagaaaaaagggaccaaaact

SEQ ID NO: 71 - DNA, 121 bp, Capsicum annuum
  variation 61: note = Polymorphism: LT17=G; PBC167=A
  source 1..121: mol_type = unassigned DNA
acaatagttttatctccagcactaaaatggttcagcacctgtgttctgtgaagtaatgtgracaattttgtgtaggatttccatgtacctgcaattgaatcacatattacaggcggagtta

SEQ ID NO: 72 - DNA, 101 bp, Capsicum annuum
  variation 51: note = Polymorphism: LT17=A; PBC167=T
  source 1..101: mol_type = unassigned DNA
ctaataatcaagatgagaatattaatatggtgcaatcagcggatgatgatwtacaaatgttgcagcagcagcctactgattttgacccaatggaggatact

SEQ ID NO: 73 - DNA, 121 bp, Capsicum annuum
  variation 61: note = Polymorphism: LT17=A; PBC167=C
  source 1..121: mol_type = unassigned DNA
gtgaagttatggaaataaatgagtagaggatctgatgtcggagcaagtagggtaaagagtmtcgcgtctcatgttgtagtgtattgtcatcataaatttcgatttttctgcttcttagtca

SEQ ID NO: 74 - DNA, 121 bp, Capsicum annuum
  variation 61: note = Polymorphism: LT17=T; PBC167=C
  source 1..121: mol_type = unassigned DNA
ctcaaatagccgggaattcattacattttccccttgttataacattatcaatactgagctyctattcttgactgactatgcactcaggtaggaattcatacatacaaactccccaaaaagg

SEQ ID NO: 75 - DNA, 101 bp, Capsicum annuum; identical to SEQ ID NO: 70
  variation 51: note = Polymorphism: LT17=C; PBC167=T
  source 1..101: mol_type = unassigned DNA

(19) United States
(12) Patent Application Publication
(10) Pub. No.: US 2025/0280784 A1
(43) Pub. Date: Sep. 11, 2025

(54) RESISTANCE TO LEVEILLULA TAURICA IN PEPPER

(71) Applicant: VILMORIN & CIE, Paris (FR)

(72) Inventors: Nicolas BARDOL, Loire-Authion (FR); Julie LEDERER, Saint Rémy de Provence (FR); Sylvie MANIN, Ledenon (FR); Benoît GORGUET, Chappes (FR)

(73) Assignee: VILMORIN & CIE, Paris (FR)

(21) Appl. No.: 18/294,738

(22) PCT Filed: Aug. 5, 2022
(86) PCT No.: PCT/EP2022/072057

§ 371 Date: Feb. 2, 2024

(30) Foreign Application Priority Data
Aug. 6, 2021 (EP) 21306101.3

(51) Int. Cl. A01H 6/82; A01H 1/00; A01H 1/02; A01H 1/04; A01H 1/06; C12N 5/04

(52) U.S. Cl. CPC A01H 6/822; A01H 1/021; A01H 1/045; A01H 1/06; A01H 1/1255; C12N 5/04

(57) ABSTRACT

A Capsicum plant resistant to powdery mildew caused by the fungal pathogen Leveillula taurica, a cell, plant part or seed of the Capsicum plant and to methods for identifying it, and the use of the plants for improving the yield of pepper production in an environment infected by Leveillula taurica and/or for limiting or controlling an infection by Leveillula taurica; wherein, the Capsicum plant includes introgressed in its genome a quantitative trait locus (QTL) conferring resistance to powdery mildew, wherein the QTL is located on chromosome 6 within the genomic interval delimited by the marker PE-0014628 (SEQ ID NO: 1) and the marker PE-0021476 (SEQ ID NO: 37).

Pepper (Capsicum sp.), from the Solanaceae family, is one of the most important vegetable crops. Whilst its species are native to the Americas, it is now widely cultivated throughout the warm, temperate, tropical and subtropical countries, in field or greenhouse production. Pepper production is affected by a variety of diseases, such as anthracnose (C. acutatum), Phytophthora blight (Phytophthora capsici), bacterial spot (Xanthomonas campestris pv. vesicatoria), and bacterial wilt (Ralstonia solanacearum). Powdery mildew is one the most important diseases of pepper. The initial symptoms of powdery mildew on peppers are a powdery-white fungal growth on the undersides of leaves with light-green to yellow blotches on the upper leaf surfaces. These areas turn brown with time, and the affected areas coalesce, causing a general yellowing of the entire leaf. The outer edges of leaves may curl upward. The older leaves lower in the canopy are usually infected first and show symptoms before the younger leaves. Infected leaves drop from the plant prematurely. This loss of photosynthetic leaf area slows plant growth and fruit development. The defoliation also exposes fruit to direct sunlight, which can lead to sunscalding of the fruit.

Leveillula taurica, the fungus that causes powdery mildew on pepper, is an obligate parasite that needs living host tissue to grow and reproduce. The fungus survives during periods between pepper crops on alternate host species. L. taurica is somewhat unusual for a powdery mildew fungus in that it has a very wide host range. The pathogen can infect over 700 species of plants in 59 different families. Susceptible crops and horticultural plants include alfalfa, artichoke, cotton, eggplant, onion, tomato, mesquite, and monkey-flower. Susceptible weed species include sow-thistle, cocklebur, groundsel, desert tobacco, and shepherd's purse. There appears to be some host specificity among isolates of the pathogen as individual isolates may be able to infect some reported host species but not others.

The pathogen is disseminated as spores carried in the wind. Powdery mildew occurs on peppers in both humid and arid environments, as the spores can germinate over the range of relative humidity levels from zero to 100%. However, the highest rates of spore germination occur when nighttime humidity levels are between 90% and 95% and daytime levels are above 85%. The spores can germinate at temperatures from 10° C. to 35° C. with the optimum temperature range is from 15° C. to 25° C. Once germination and infection have occurred, disease development is favored by warm days and cool nights, with the most severe disease levels occurring with daytime temperatures near 30° C. and nighttime temperatures below 25° C. Disease development is suppressed at temperatures above 35° C. While infection is highest under humid conditions, defoliation is favored by drier conditions. Therefore, the impact of the disease is most severe when humidity levels change from humid to dry.

Yield reductions from powdery mildew result, in part, from a loss of photosynthetic area that inhibits plant growth and fruit development. Fewer flowers form on severely infected plants, and flowers may also drop off of infected plants. Additional losses come from sunscalding of the fruit.

With the wide host range of the pathogen and wind dispersal of the spores, crop rotation and management of nearby weed hosts may not provide an adequate level of control without using other means of control. Low light and high humidity favor the development of powdery mildew on peppers, so cultural practices that minimize these conditions can help lower disease severity. Such practices include the selection of sites with well-drained soils and good air circulation; the adjustment of planting densities and row orientations to promote good air circulation and light penetration into the canopy; or the avoidance of excessive fertilization that would result in overly dense canopies. In greenhouse production, increasing the temperature in the greenhouse can lower humidity levels and slow disease development.

A second control method for powdery mildew on peppers is the application of fungicides. Several fungicides are available for controlling powdery mildew on peppers, although chemical control may be hampered by the semi-endoparasitic nature of the fungi. Furthermore, the use of fungicides should be accompanied by appropriate fungicide resistance management strategies to prevent the development of fungicide-resistant strains of the pathogen. In addition, the ban on certain chemical treatments, or their social perception, reduces the ability of agronomic control of the disease.

It is thus desirable to find new pepper germplasms having a moderate to high resistant to the disease. Some sources of resistance have been identified, although most of them showed a low level of resistance. Daubeze et al, 1995 identified a resistant Ethiopian line H3. Lefebvre et al 2003 analyzed doubled haploid lines derived from a cross between H3 and the susceptible line Vania. The plants were evaluated in two different infections conditions and five QTLs distributed on five distinct chromosomes, P5, P6, P9, P10 and P12 were detected, with additive effects and epistatic interactions. However, some of the already identified QTLs may be linked to undesirable traits and/or provide only a partial resistance to the pathogen. Furthermore, the effect of QTLs may be additive, such that there is an interest in finding new QTLs, the effect of which could be cumulated to improve the resistance to the pathogen. There remains thus a strong need to find QTLs and sources of resistance to Leveillula taurica in pepper.

### SUMMARY OF THE INVENTION

The present invention first provides a Capsicum plant resistant to powdery mildew, comprising introgressed in its genome a quantitative trait locus (QTL) conferring resistance to powdery mildew, wherein said QTL is present on chromosome 6.

In some aspects, the invention provides a Capsicum plant resistant to powdery mildew, comprising introgressed in its genome a quantitative trait locus (QTL) conferring resistance to powdery mildew, wherein said QTL is located on chromosome 6 within the chromosomal region delimited by the markers PE-0014628 (SEQ ID NO: 1) and PE-0021476 (SEQ ID NO: 37).

In some aspects, the invention provides a Capsicum plant resistant to powdery mildew, comprising introgressed in its genome a quantitative trait locus (QTL) conferring resistance to powdery mildew, wherein said QTL is present on chromosome 6 in the genome of a seed of Capsicum annuum ST17-1157, deposited at NCIMB under accession number NCIMB 43602.

The present invention also provides a Capsicum plant resistant to powdery mildew, comprising introgressed in its genome a quantitative trait locus (QTL) conferring resistance to powdery mildew, wherein the presence of said QTL on chromosome 6 and conferring said resistance is characterized by at least one allele selected from the group consisting of allele A of PE-0056543 (SEQ ID NO: 9), allele A of PE-0056544 (SEQ ID NO:13), allele C of PE-0056545 (SEQ ID NO: 17) and allele C of PE-0056546 (SEQ ID NO: 21).

The present invention also provides a Capsicum plant resistant to powdery mildew, comprising introgressed in its genome a quantitative trait locus (QTL) conferring resistance to powdery mildew, wherein said QTL encompasses the CA06g26150 gene and/or the CA06g26250 gene, in particular the CA06g26150 gene.

The present invention also provides a Capsicum plant resistant to powdery mildew, wherein said Capsicum plant comprises a mutant allele of the CA06g26150 gene and/or the CA06g26250 gene, in particular the CA06g26150 gene, wherein said mutant allele confers resistance to powdery mildew.

The present invention also provides a Capsicum seed, which can be grown into a Capsicum plant according to the invention.

Also provided is an in vitro cell or tissue culture of regenerable cells of the Capsicum plant according to the invention, wherein the regenerable cells are derived from an embryo, protoplast, meristematic cell, callus, pollen, leaf, anther, stem, petiole, root, root tip, seed, flower, cotyledon, and/or hypocotyl.

Further provided is a method for detecting and/or selecting Capsicum plants comprising in their genome a QTL on chromosome 6 as found in the genome of Capsicum annuum ST17-1157 deposited at the NCIMB under accession number NCIMB 43602, said QTL conferring resistance to powdery mildew, comprising the detection of at least one of the following alleles: allele A of PE-0056543 (SEQ ID NO: 9), allele A of PE-0056544 (SEQ ID NO:13), allele C of PE-0056545 (SEQ ID NO: 17) and allele C of PE-0056546 (SEQ ID NO: 21).

Also provided is the use of a plant or seed of ST17-1157, deposited at NCIMB under accession number NCIMB-43602, or progeny thereof bearing the sequences conferring the resistance to powdery mildew, as a breeding partner in a breeding program for obtaining a Capsicum plant resistant to powdery mildew.

The invention also relates to a method for producing a Capsicum plant resistant to powdery mildew, comprising the steps of
(a) crossing a plant grown from a seed of ST17-1157 deposited at NCIMB under accession number NCIMB 43602 or progeny thereof bearing the QTL conferring the resistance to powdery mildew, and a susceptible Capsicum plant,
(b) selecting one plant resistant to powdery mildew in the progeny thus obtained, said plant bearing the QTL conferring the resistance to powdery mildew;
(c) optionally self-pollinating one or several times the resistant plant obtained at step b) and selecting a plant resistant to powdery mildew in the progeny thus obtained;
(d) optionally backcrossing the resistant plant selected in step b) or c) with a susceptible Capsicum plant and
(e) selecting a plant resistant to powdery mildew,
   wherein selecting plants resistant to powdery mildew in steps b), c) and/or e) comprises the detection of nucleic acid markers, in particular SNP markers.

Further provided is a method for producing Capsicum plants resistant to powdery mildew, comprising the steps of:
(a1) crossing a plant grown from a seed deposited under accession number NCIMB 43602 or progeny thereof bearing the sequences conferring the resistance to powdery mildew and a susceptible Capsicum plant, thus generating the F1 population,
(a2) advancing the F1 population to create F2 population,
(b) selecting one resistant plant in the progeny thus obtained;
c) optionally self-pollinating one or several times the resistant plant obtained at step b) and selecting a plant resistant to white rust in the progeny thus obtained;
(d) optionally backcrossing the resistant plant selected in step b) or c) with a susceptible Capsicum plant, and
(e) selecting a plant resistant to powdery mildew,
   wherein selecting plants resistant to powdery mildew in steps b), c) and/or e) comprises the detection of nucleic acid markers, in particular SNP markers.

Also provided is a method for obtaining Capsicum plants of agricultural interest, resistant to powdery mildew, comprising the steps of:
(a) backcrossing a plant obtained by germinating a seed deposited at the NCIMB under accession number NCIMB 43602 or progeny thereof bearing the sequences conferring the resistance to powdery mildew, with a Capsicum plant susceptible to powdery mildew,
(b) Selecting a plant resistant to powdery mildew, wherein the selection comprises the detection of nucleic acid markers, in particular SNP markers.

The invention also relates to a Capsicum plant resistant to powdery mildew obtainable by a method or use according to the invention. Said Capsicum plant comprises a QTL on chromosome 6 as defined herein, conferring the resistance to powdery mildew.

The invention further concerns a method for identifying a molecular marker linked with a QTL conferring the resistance to powdery mildew, said QTL being present in seeds deposited at the NCIMB under accession number NCIMB 43602, comprising:
(a) identifying a molecular marker in Capsicum genome, in the chromosomal region delimited on chromosome 6 by PE-0014628 (SEQ ID NO: 1) and PE-0021476 (SEQ ID NO: 37); and
(b) determining whether an allele or state of said molecular marker is associated with resistance to powdery mildew in a segregating population comprising Capsicum plants exhibiting said resistance.

Also provided is a method for improving the yield of pepper production in an environment infected by Leveillula taurica, comprising growing a Capsicum plant according to the invention.

Further provided is a method for protecting a field, tunnel, greenhouse or glasshouse of pepper from an infection by Leveillula taurica, comprising growing a Capsicum plant according the invention.

The invention also relates to the use of a Capsicum sp. plant according to according to the invention for limiting or controlling an infection by Leveillula taurica.

Another embodiment of the invention relates to a method of producing pepper fruit comprising:
a) growing a Capsicum plant according to the invention;
b) allowing said plant to set fruit; and
c) harvesting fruit of said plant, preferably at maturity and/or over-maturity; and optionally
d) processing said pepper fruit into a pepper processed food.

### Definitions

As used herein, the term “pepper”, “pepper plant” or “Capsicum plant” relates to any species, variety, cultivar, or population of the Capsicum genus. The Capsicum genus is known to comprise 20-27 species, five of which are domesticated: Capsicum annuum, Capsicum baccatum, Capsicum frutescens, Capsicum chinense, and Capsicum pubescens. The vast majority of commercial varieties of peppers belong the species Capsicum annuum, which comprises sweet peppers (peppers with a no pungency) and hot peppers (peppers with pungency from low level to high level). Sweet peppers include bell peppers, which are fruit of plants in the Grossum cultivar group of the species Capsicum annuum. Sweet pepper plants can be produced in different colors, from immature green color to mature color of red, yellow, orange, white or purple. In particular, the Capsicum plant may be of one of the following types: Dulce Italiano, Lamuyo, Blocky Florida, Open field and blocky protected, Ancho, Anaheim, Marconi, Jalapeno, Cayenne, Charleston or Sivri.

As used herein, the term “plant part” refers to any part of a plant including but not limited to the shoot, root, stem, seeds, fruits, leaves, petals, flowers, ovules, branches, petioles, internodes, pollen, stamen, rootstock, scion and the like.

As used herein, the term “allele” refers to any of several alternative or variant forms of a genetic unit, such as a gene, which are alternative in inheritance because they are positioned at the same locus in homologous chromosomes. Such alternative or variant forms may be the result of single nucleotide polymorphisms, insertions, inversions, translocations or deletions, or the consequence of gene regulation caused, for example, by chemical or structural modification, transcription regulation or post-translational modification/regulation. In a diploid cell or organism such as pepper, the two alleles of a given gene or genetic element typically occupy corresponding loci on a pair of homologous chromosomes.

As used herein, the term “genotype” refers to the genetic makeup of an individual cell, cell culture, tissue, organism (e.g., a plant), or group of organisms.

As used herein, the term “locus” (plural: “loci”) refers to any site that has been defined genetically, this can be a si (Single Nucleotide Polymorphism), by several markers (e.g. SNPs), or by two flanking markers (e.g. SNPs).

As used herein, the terms “Quantitative Trait Locus (QTL)” (plural: “Quantitative Trait Loci”) refer to a genomic region that may comprise one or more genes or regulatory sequences. A QTL may for instance comprise one or more genes of which products confer genetic resistance or tolerance. Alternatively, a QTL may for instance comprise regulatory genes or sequences of which products influence the expression of genes on other loci in the genome of the plant thereby conferring the resistance or tolerance. The QTL of the present invention may be defined by indicating its genetic location in the genome of the respective pathogen-resistant accession using one or more molecular genomic markers. One or more markers, in turn, indicate a specific locus. Distances between loci are usually measured by frequency of crossing-over between loci on the same chromosome. The farther apart two are, the more likely that a crossover will occur between them. Conversely, if two loci are close together, a cross over is less likely to occur between them. As a rule, one centimorgan (cM) is equal to 1% recombination between loci (marker). When a QTL can be indicated by multiple markers, the genetic distance between the end-point markers is indicative of the size of the QTL.

The term “resistance” is as defined by the ISF (International Seed Federation) Vegetable and Ornamental Crops Section for describing the reaction of plants to pests or pathogens, and abiotic stresses for the Vegetable Seed Industry. Specifically, by resistance, it is meant the ability of a plant variety to restrict the growth and development of a specified pest or pathogen and/or the damage they cause when compared to susceptible plant varieties under similar environmental conditions and pest or pathogen pressure. Resistant varieties may exhibit some disease symptoms or damage under heavy pest or pathogen pressure.

By “tolerance” is meant the ability of a plant variety to endure biotic and abiotic stress without serious consequences for growth, appearance and yield.

As used herein, the term “susceptible” refers to a plant that is unable to restrict the growth and development of a specified pest or pathogen.

As used herein, the term “offspring” or “progeny” refers to any plant resulting as progeny from a vegetative or sexual reproduction from one or more parent plants or descendants thereof. For instance, an offspring plant may be obtained by cloning or selfing of a parent plant or by crossing two parent plants and include selfings as well as the F1 or F2 or still further generations. An F1 is a first-generation offspring produced from parents at least one of which is used for the first time as donor of a trait, while offspring of a second generation (F2) or subsequent generations (F3, F4, etc.) are specimens produced from selfing of F1's, F2s, etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

As used herein, the terms “cross”, “crossing” refer to the process by which the pollen of one flower on one plant is applied (artificially or naturally) to the ovule (stigma) of a flower on another plant.

As used herein, the term “heterozygote” refers to a diploid or polyploidy cell or plant having different alleles (forms of a given gene or sequences) present at at least one locus.

As used herein, the term “heterozygous” refers to the presence of different alleles (forms of a given gene or sequences) at a particular locus.

As used herein, the term “homozygote” refers to an individual cell or plant having the same alleles at one or more loci on all homologous chromosomes.

As used herein, the term “homozygous” refers to the presence of identical alleles at one or more loci in homologous chromosomal segments.

As used herein, the term “hybrid” refers to any individual cell, tissue or plant resulting from a cross between parents that differ in one or more genes.

As used herein, the term “inbred” or “line” refers to a relatively true-breeding strain.

As used herein, the term “phenotype” refers to the observable characters of an individual cell, cell culture, organism (e.g. a plant), or group of organisms which results from the interaction between that individual genetic makeup (i.e. genotype) and the environment.

As used herein, the terms “introgression”, “introgressed” and “introgressing” refer to the process whereby genes of one species, variety or cultivar are moved into the genome of another species, variety or cultivar, by crossing those species. The crossing may be natural or artificial. The process may be optionally be completed by backcrossing to the recurrent parent, in which case introgression refers to infiltration of the genes of one species into the gene pool of another through repeated backcrossing of an interspecific hybrid with one of its parents. An introgression may be also described as a heterologous genetic material stably integrated in the genome of a recipient plant.

As used herein, the terms “molecular marker” refer to an indicator that is used in methods for visualizing differences in characteristics of nucleic acid sequences. Examples of such indicators are restriction fragment length polymorphism (RFLP) markers, amplification fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Mapping of molecular markers in the vicinity of an allele is a procedure which can be performed quite easily by the person skilled in the art using common molecular techniques.

As used herein, “marker-based selection” or “marker-assisted selection (MAS)” or “marker-assisted breeding [symbol] undesirable) traits, so that those plants can be used (or avoided) in a selective breeding program.

As used herein, the term “primer” refers to an oligonucleotide which is capable of annealing to the amplification target allowing a DNA polymerase to attach, thereby serving as a point of initiation of DNA synthesis when placed under conditions in which synthesis of primers extension product is induced, i.e., in the presence of nucleotides and an agent for polymerization such as DNA polymerase and at a suitable temperature and pH. The primer is preferably single stranded for maximum efficiency in amplification. Preferably, the primer is an oligodeoxyribonucleotide. The primer must be sufficiently long to prime the synthesis of extension products in the presence of the agent for polymerization. The exact length of the primers will depend on many factors, including temperature and composition (A/T and G/C content) of primer. A pair of bi-directional primers consists of one forward and one reverse primer as commonly used in the art of DNA amplification such as in PCR amplification.

As used herein, a single nucleotide polymorphism (SNP) is a DNA sequence variation occurring when a single AAGCCTA to AAGCTTA, contain a difference in a single nucleotide. In this case there are two alleles: C and T.

In the context of the invention and unless otherwise specified, DNA strand and allele designation and orientation for the markers (i.e. SNPs) disclosed in the present application are mentioned according to the TOP/BOT method developed by Illumina (https://www.illumina.com/documents/products/technotes/technote_topbot.pd).

By “powdery mildew”, it is meant a disease which primarily affects leaves on pepper plants. Symptoms include patchy, white, powdery growth, called sporulation, that enlarges and coalesces to cover the entire lower leaf surface. At times the powdery growth is present on the upper leaf surface as well. Powdery mildew is caused in pepper by the fungal pathogen Leveillula taurica.

In the context of the present application, a chromosomal region delimited by two markers X and Y (e.g. SNPs) refers to the section of the chromosome lying between the positions of these two markers and comprising said markers, therefore the nucleotide sequence of this chromosomal region begins with the nucleotide corresponding to marker X and ends with the nucleotide corresponding to marker Y, i.e. the markers are comprised within the region they delimit.

By association, or genetic association, and more specifically genetic linkage, it is to be understood that a polymorphism of a genetic marker (e.g. a specific allele of the SNP marker) and the phenotype of interest occur simultaneously, i.e. are inherited together, more often than would be expected by chance occurrence, i.e. there is a non-random association of the allele and of the genetic sequences responsible for the phenotype, as a result of their genomic proximity.

### SEQUENCE LISTING

SEQ ID NO: 1 shows a sequence surrounding the marker PE-0014628.

SEQ ID NO: 2 shows the sequence of a forward primer for detecting the susceptible allele of PE-0014628.

SEQ ID NO: 3 shows the sequence of a forward primer for detecting the resistant allele of PE-0014628.

SEQ ID NO: 4 shows the sequence of a common reverse primer for PE-0014628.

SEQ ID NO: 5 shows a sequence surrounding the marker PE-0056542.

SEQ ID NO: 6 shows the sequence of a forward primer for detecting the susceptible allele of PE-0056542.

SEQ ID NO: 7 shows the sequence of a forward primer for detecting the resistant allele of PE-0056542.

SEQ ID NO: 8 shows the sequence of a common reverse primer for PE-0056542.

SEQ ID NO: 9 shows a sequence surrounding the marker PE-0056543.

SEQ ID NO: 10 shows the sequence of a forward primer for detecting the susceptible allele of PE-0056543.

SEQ ID NO: 11 shows the sequence of a forward primer for detecting the resistant allele of PE-0056543.

SEQ ID NO: 12 shows the sequence of a common reverse primer for PE-0056543.

SEQ ID NO: 13 shows a sequence surrounding the marker PE-0056544.

SEQ ID NO: 14 shows the sequence of a forward primer for detecting the susceptible allele of PE-0056544.

SEQ ID NO: 15 shows the sequence of a forward primer for detecting the resistant allele of PE-0056544.

SEQ ID NO: 16 shows the sequence of a common reverse primer for PE-0056544.

SEQ ID NO: 17 shows a sequence surrounding the marker PE-0056545.

SEQ ID NO: 18 shows the sequence of a forward primer for detecting the susceptible allele of PE-0056545.

SEQ ID NO: 19 shows the sequence of a forward primer for detecting the resistant allele of PE-0056545.

SEQ ID NO: 20 shows the sequence of a common reverse primer for PE-0056545.

SEQ ID NO: 21 shows a sequence surrounding the marker PE-0056546.

SEQ ID NO: 22 shows the sequence of a forward primer for detecting the susceptible allele of PE-0056546.

SEQ ID NO: 23 shows the sequence of a forward primer for detecting the resistant allele of PE-0056546.

SEQ ID NO: 24 shows the sequence of a common reverse primer for PE-0056546.

SEQ ID NO: 25 shows a sequence surrounding the marker PE-0056547.

SEQ ID NO: 26 shows the sequence of a forward primer for detecting the susceptible allele of PE-0056547.

SEQ ID NO: 27 shows the sequence of a forward primer for detecting the resistant allele of PE-0056547.

SEQ ID NO: 28 shows the sequence of a common reverse primer for PE-0056547.

SEQ ID NO: 29 shows a sequence surrounding the marker PE-0056549.

SEQ ID NO: 30 shows the sequence of a forward primer for detecting the susceptible allele of PE-0056549.

SEQ ID NO: 31 shows the sequence of a forward primer for detecting the resistant allele of PE-0056549.

SEQ ID NO: 32 shows the sequence of a common reverse primer for PE-0056549.

SEQ ID NO: 33 shows a sequence surrounding the marker PE-0056550.

SEQ ID NO: 34 shows the sequence of a forward primer for detecting the susceptible allele of PE-0056550.

SEQ ID NO: 35 shows the sequence of a forward primer for detecting the resistant allele of PE-0056550.

SEQ ID NO: 36 shows the sequence of a common reverse primer for PE-0056550.

SEQ ID NO: 37 shows a sequence surrounding the marker PE-0021476.

SEQ ID NO: 38 shows the sequence of a forward primer for detecting the susceptible allele of PE-0021476.

SEQ ID NO: 39 shows the sequence of a forward primer for detecting the resistant allele of PE-0021476.

SEQ ID NO: 40 shows the sequence of a common reverse primer for PE-0021476.

SEQ ID NO: 41 shows a coding sequence of the CA06g26150 gene in a susceptible plant (Maor).

SEQ ID NO: 42 shows a coding sequence of the CA06g26250 gene in a susceptible plant (Maor).

SEQ ID NO: 43 shows the sequence of a forward primer for amplifying the susceptible allele of the RZ marker.

SEQ ID NO: 44 shows the sequence of a forward primer for amplifying the resistant allele of the RZ marker.

SEQ ID NO: 45 shows the sequence of a common reverse primer for amplifying the resistant or susceptible allele of the RZ marker.

SEQ ID NOs: 46 to 75 show sequences surrounding, respectively markers PE-0011447, PE-0011448, PE-0019751, PE-0021027, PE-0022402, PE-0018388, PE-0028409, PE-0006573, PE-0001206, PE-0005987, PE-0005367, PE-0006634, PE-0006718, PE-0012892, PE-0004191, PE-0005575, PE-0006320, PE-0016499, PE-0013625, PE-0022671, PE-0005787, PE-0020909, PE-0000401, PE-0003984, PE-0014672, PE-0005702, PE-0021582, PE-0007248, PE-0003669, PE-0014672.

### LEGEND OF THE FIGURES

FIG. 1 shows a genetic distance tree showing, inter alia, the genetic distance of line LT17 with respect to other Capsicum accessions.

FIG. 2 shows the distribution of the AUDPC (Area under the disease progress curve) for the parameter “Global note” measured on a segregating F2 population from the cross between the susceptible line Maor and the LT17 Line.

FIG. 3 shows the logarithm of odds (LOD), across chromosome 6 of the pepper genome, for the phenotype of resistance to Leveillula taurica.

FIG. 4 shows the distribution of disease scoring function to the presence of the QTL on chromosome 6, in two test locations.

FIG. 5: Panel (A) shows the disease scoring in different plant populations: S Check 3 (sensible control), S check (sensible control), IS check (Intermediate sensible control), Parent S (sensible), QTL6 SS (homozygous sensible at QTL6), QTL6 RS (heterozygous at QTL6), QTL6 RR (homozygous at QTL6), F4-RR-deposit-2 (F4 population ST117F04-1157/BLK homozygous resistant at QTL6), R check (resistant control) and Parent R (resistant parent). Panel (B) shows the result of a Tukey's test on the disease scoring in the same different plant populations.

### DETAILED DESCRIPTION OF THE INVENTION

According to a first aspect, the present invention is directed to a Capsicum plant resistant to powdery mildew caused by the fungal pathogen Leveillula taurica, comprising introgressed in its genome a quantitative trait locus (QTL) on chromosome 6, conferring resistance to powdery mildew.

In some embodiments, said QTL is homozygously present in the genome of the plant.

In some embodiments, said QTL is heterozygously present in the genome of the plant.

In some embodiments, said QTL is present on chromosome 6 in the genome of a seed of Capsicum annuum ST17-1157, deposited at NCIMB accession number NCIMB 43602.

In some embodiments, said QTL is located on chromosome 6 within the chromosomal region delimited by markers PE-0014628 (SEQ ID NO: 1) and PE-0021476 (SEQ ID NO: 37). In some embodiments, said QTL is located on chromosome 6 within the chromosomal region delimited by positions 232015212 and 235235179 on the public genome Capsicum annuum cv CM334 v.1.55 (accessible on http://www.solgenomics.net). In particular, said QTL can be located on chromosome 6 within the chromosomal region delimited by markers PE-0056542 (SEQ ID NO: 5) and PE-0056550 (SEQ ID NO: 33). More particularly, said QTL is located on chromosome 6 within the chromosomal region delimited within the chromosomal region delimited by PE-0014628 (SEQ ID NO: 1) and PE-0056550 (SEQ ID NO: 33). More specifically, said QTL is located on chromosome 6 within the chromosomal region delimited by PE-0056542 (SEQ ID NO: 5) and PE-0021476 (SEQ ID NO: 37).

In some embodiments, said QTL on chromosome 6 conferring resistance to powdery mildew is identified by at least one marker selected from the group consisting of PE-0014628 (SEQ ID NO: 1), PE-0056542 (SEQ ID NO: 5), PE-0056543 (SEQ ID NO: 9), PE-0056544 (SEQ ID NO: 13), PE-0056545 (SEQ ID NO: 17), PE-0056546 (SEQ ID NO: 21), PE-0056547 (SEQ ID NO: 25), PE-0056549 (SEQ ID NO: 29), PE-0056550 (SEQ ID NO: 33) and PE-0021476 (SEQ ID NO: 37), preferably at least one marker selected from the group consisting of PE-0056543 (SEQ ID NO: 9), PE-0056544 (SEQ ID NO: 13), PE-0056545 (SEQ ID NO: 17), PE-0056546 (SEQ ID NO: 21), PE-0056547 (SEQ ID NO: 25), PE-0056549 (SEQ ID NO: 29) and PE-0056550 (SEQ ID NO: 33), or any other marker comprised within the chromosomal region delimited by PE-0056542 (SEQ ID NO: 5) and PE-0056550 (SEQ ID NO: 33).

In some embodiments, said QTL on chromosome 6 conferring resistance to powdery mildew is identified by detection of the PE-0014628 marker on chromosome 6. In particular, said detection of the PE-0014628 marker on chromosome 6 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 1, or a fragment thereof including the [A/G] polymorphism at position 51 of SEQ ID NO: 1. For example, the forward primer for detecting the susceptible allele of the PE-0014628 marker, by amplifying a nucleic acid consisting of sequence SEQ ID NO: 1 or its complementary sequence, may consist of the sequence 5′-GAAGGTCGGAGTCAACGGATTAGAAGAATAAGCACGAATAACCGCG-3′ (SEQ ID NO: 2), the forward primer for detecting the resistant allele of the PE-0014628 marker, by amplifying a nucleic acid consisting of sequence SEQ ID NO: 1 or its complementary sequence, may consist of the sequence 5′-GAAGGTGACCAAGTTCATGCTAGAAGAATAAGCACGAATAACCGCA-3′ (SEQ ID NO: 3), and the common reverse primer may consist of the sequence 5′-AATTCCTCAACCAAATCTATTTTCTTGGAA-3′ (SEQ ID NO: 4). Using primers consisting of sequences SEQ ID NO: 2, SEQ ID NO: 3 and SEQ ID NO: 4, detection of an alanine (A) rather than a guanine (G), at position 51 of the sequence SEQ ID NO: 1, or a thymine (T) rather than a cytosine (C) in the complementary strand, indicates the presence of the QTL on chromosome 6 conferring resistance to powdery mildew (see Table 1 below).

In some embodiments, said QTL on chromosome 6 conferring resistance to powdery mildew is identified by detection of the PE-0056542 marker on chromosome 6. In particular, detection of the PE-0056542 marker on chromosome 6 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 5, or a fragment thereof including the [T/C] polymorphism at position 151 of SEQ ID NO: 5. For example, the forward primer for detecting the susceptible allele of the PE-0056542 marker, by amplifying a nucleic acid consisting of sequence SEQ ID NO: 5 or its complementary sequence, may consist of the sequence 5′-GAAGGTCGGAGTCAACGGATTGTGGTTATGACTGGGCCCCTT-3′ (SEQ ID NO: 6), the forward primer for detecting the resistant allele of the PE-0056542 marker, by amplifying a nucleic acid consisting of sequence SEQ ID NO: 5 or its complementary sequence, may consist of the sequence 5′-GAAGGTGACCAAGTTCATGCTGTGGTTATGACTGGGCCCCTC-3′ (SEQ ID NO: 7), and the common reverse primer may consist of the sequence 5′-TAAGAGCTCTGTTGTCCAAAACCATCTT-3′ (SEQ ID NO: 8). Using primers consisting of sequences SEQ ID NO: 6, SEQ ID NO: 7 and SEQ ID NO: 8, detection of a cytosine (C) rather than a thymine (T) at position 151 of the sequence SEQ ID NO: 5, or a guanine (G) rather than an alanine (A) in the complementary strand, indicates the presence of the QTL on chromosome 6 conferring resistance to powdery mildew (see Table 1 below).

In some embodiments, said QTL on chromosome 6 conferring resistance to powdery mildew is identified by detection of the PE-0056543 marker on chromosome 6. In particular, detection of the PE-0056543 marker on chromosome 6 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 9, or a fragment thereof including the [G/A] polymorphism at position 151 of SEQ ID NO: 9. For example, the forward primer for detecting the susceptible allele of the PE-0056543 marker, by amplifying a nucleic acid consisting of sequence SEQ ID NO: 9 or its complementary sequence, may consist of the sequence 5′-GAAGGTCGGAGTCAACGGATTCATTAGAAGATCCAAATAATACTTTGAG-3′ (SEQ ID NO: 10), the forward primer for detecting the resistant allele of the PE-0056543 marker, by amplifying a nucleic acid consisting of sequence SEQ ID NO: 9 or its complementary sequence, may consist of the sequence 5′-GAAGGTGACCAAGTTCATGCTAGATTCATTAGAAGATCCAAATAATACTTTGAA-3′ (SEQ ID NO: 11), and the common reverse primer may consist of the sequence 5′-ATGAAACCTTCCGTCAGATTATCGAACTT-3′ (SEQ ID NO: 12). Using primers consisting of sequences SEQ ID NO: 10, SEQ ID NO: 11 and SEQ ID NO: 12, detection of an alanine (A) rather than a guanine (G) at position 151 of the sequence SEQ ID NO: 9, or a thymine (T) rather than a cytosine (C) in the complementary strand, indicates the presence of the QTL on chromosome 6 conferring resistance to powdery mildew (see Table 1 below).

In some embodiments, said QTL on chromosome 6 conferring resistance to powdery mildew is identified by detection of the PE-0056544 marker on chromosome 6. In particular, detection of the PE-0056544 marker on chromosome 6 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 13, or a fragment thereof including the [G/T] polymorphism at position 151 of SEQ ID NO: 13. For example, the forward primer for detecting the susceptible allele of the PE-0056544 marker, by amplifying a nucleic acid consisting of sequence SEQ ID NO: 13 or its complementary sequence, may consist of the sequence 5′-GAAGGTGACCAAGTTCATGCTGGTGCTGTATTTTTGGCCATCGG-3′ (SEQ ID NO: 14), the forward primer for detecting the resistant allele of the PE-0056544 marker, by amplifying a nucleic acid consisting of sequence SEQ ID NO: 13 or its complementary sequence, may consist of the sequence 5′-GAAGGTCGGAGTCAACGGATTAGGTGCTGTATTTTTGGCCATCGT-3′ (SEQ ID NO: 15), and the common reverse primer may consist of the sequence 5′-TTCCTCTTCCTGGATATCCTTCTCATATA-3′ (SEQ ID NO: 16). Using primers consisting of sequences SEQ ID NO: 14, SEQ ID NO: 15 and SEQ ID NO: 16, detection of a thymine (T) rather than a guanine (G) at position 151 of the sequence SEQ ID NO: 13, or an alanine (A) rather than a cytosine (C) in the complementary strand, indicates the presence of the QTL on chromosome 6 conferring resistance to powdery mildew (see Table 1 below).

In some embodiments, said QTL on chromosome 6 conferring resistance to powdery mildew is identified by detection of the PE-0056545 marker on chromosome 6. In particular, detection of the PE-0056545 marker on chromosome 6 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 17, or a fragment thereof including the [T/C] polymorphism at position 151 of SEQ ID NO: 17. For example, the forward primer for detecting the susceptible allele of the PE-0056545 marker, by amplifying a nucleic acid consisting of sequence SEQ ID NO: 17 or its complementary sequence, may consist of the sequence 5′-GAAGGTCGGAGTCAACGGATTCTGTCAGTGGCCTTCATAAGATCA-3′ (SEQ ID NO: 18), the forward primer for detecting the resistant allele of the PE-0056545 marker, by amplifying a nucleic acid consisting of sequence SEQ ID NO: 17 or its complementary sequence, may consist of the sequence 5′-GAAGGTGACCAAGTTCATGCTGTCAGTGGCCTTCATAAGATCG-3′ (SEQ ID NO: 19), and the common reverse primer may consist of the sequence 5′-GTTTGAGAAGTCTACTTCAAAAATGAGATT-3′ (SEQ ID NO: 20). Using primers consisting of sequences SEQ ID NO: 18, SEQ ID NO: 19 and SEQ ID NO: 20, detection of a cytosine (C) rather than a thymine (T) at position 151 of the sequence SEQ ID NO: 17, or a guanine (G) rather than an alanine (A) in the complementary strand, indicates the presence of the QTL on chromosome 6 conferring resistance to powdery mildew (see Table 1 below).

In some embodiments, said QTL on chromosome 6 conferring resistance to powdery mildew is identified by detection of the PE-0056546 marker on chromosome 6. In particular, detection of the PE-0056546 marker on chromosome 6 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 21, or a fragment thereof including the [A/C] polymorphism at position 151 of SEQ ID NO: 21. For example, the forward primer for detecting the susceptible allele of the PE-0056546 marker, by amplifying a nucleic acid consisting of sequence SEQ ID NO: 21 or its complementary sequence, may consist of the sequence 5′-GAAGGTGACCAAGTTCATGCTCCTTCACTCACCGAATGTAACCT-3′ (SEQ ID NO: 22), the forward primer for detecting the resistant allele of the PE-0056546 marker, by amplifying a nucleic acid consisting of sequence SEQ ID NO: 21 or its complementary sequence, may consist of the sequence 5′-GAAGGTCGGAGTCAACGGATTCCTTCACTCACCGAATGTAACCG-3′ (SEQ ID NO: 23), and the common reverse primer may consist of the sequence 5′-TTGGTCTACAAAGACATGCCAAATGGAA-3′ (SEQ ID NO: 24). Using primers consisting of sequences SEQ ID NO: 22, SEQ ID NO: 23 and SEQ ID NO: 24, detection of a cytosine (C) rather than an alanine (A) at position 151 of the sequence SEQ ID NO: 21, or a guanine (G) rather than a thymine (T) in the complementary strand, indicates the presence of the QTL on chromosome 6 conferring resistance to powdery mildew (see Table 1 below).

In some embodiments, said QTL on chromosome 6 conferring resistance to powdery mildew is identified by detection of the PE-0056547 marker on chromosome 6. In particular, detection of the PE-0056547 marker on chromosome 6 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 25, or a fragment thereof including the [A/C] polymorphism at position 151 of SEQ ID NO: 25. For example, the forward primer for detecting the susceptible allele of the PE-0056547 marker, by amplifying a nucleic acid consisting of sequence SEQ ID NO: 25 or its complementary sequence, may consist of the sequence 5′-GAAGGTGACCAAGTTCATGCTCAACTCTCCGGTGAATCTAAGCTTA-3′ (SEQ ID NO: 26), the forward primer for detecting the resistant allele of the PE-0056547 marker, by amplifying a nucleic acid consisting of sequence SEQ ID NO: 25 or its complementary sequence, may consist of the sequence 5′-GAAGGTCGGAGTCAACGGATTAACTCTCCGGTGAATCTAAGCTTC-3′ (SEQ ID NO: 27), and the common reverse primer may consist of the sequence 5′-CATTGTCATAACCTTTACCAGACAATGAAT-3′ (SEQ ID NO: 28). Using primers consisting of sequences SEQ ID NO: 26, SEQ ID NO: 27 and SEQ ID NO: 28, detection of a cytosine (C) rather than an alanine (A) at position 151 of the sequence SEQ ID NO: 25, or a guanine (G) rather than a thymine (T) in the complementary strand, indicates the presence of the QTL on chromosome 6 conferring resistance to powdery mildew (see Table 1 below).

In some embodiments, said QTL on chromosome 6 conferring resistance to powdery mildew is identified by detection of the PE-0056549 marker on chromosome 6. In particular, detection of the PE-0056549 marker on chromosome 6 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 29, or a fragment thereof including the [T/A] polymorphism at position 151 of SEQ ID NO: 29. For example, the forward primer for detecting the susceptible allele of the PE-0056549 marker, by amplifying a nucleic acid consisting of sequence SEQ ID NO: 29 or its complementary sequence, may consist of the sequence 5′-GAAGGTCGGAGTCAACGGATTCCGATATTTTATACTTAGCTATTCTAGCATAT-3′ (SEQ ID NO: 30), the forward primer for detecting the resistant allele of the PE-0056549 marker, by amplifying a nucleic acid consisting of sequence SEQ ID NO: 29 or its complementary sequence, may consist of the sequence 5′-GAAGGTGACCAAGTTCATGCTCCGATATTTTATACTTAGCTATTCTAGCATAA-3′ (SEQ ID NO: 31), and the common reverse primer may consist of the sequence 5′-GAGTTTCATGATTCATCTGTTTTGTTTCCC-3′ (SEQ ID NO: 32). Using primers consisting of sequences SEQ ID NO: 30, SEQ ID NO: 31 and SEQ ID NO: 32, detection of an alanine (A) rather than a thymine (T) at position 151 of the amplification product consisting of sequence SEQ ID NO: 29, or a thymine (T) rather than alanine (A) in the complementary strand, indicates the presence of the QTL on chromosome 6 conferring resistance to powdery mildew (see Table 1 below).

In some embodiments, said QTL on chromosome 6 conferring resistance to powdery mildew is identified by detection of the PE-0056550 marker on chromosome 6. In particular, detection of the PE-0056550 marker on chromosome 6 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 33, or a fragment thereof including the [C/T] polymorphism at position 151 of SEQ ID NO: 33. For example, the forward primer for detecting the susceptible allele of the PE-0056550 marker, by amplifying a nucleic acid consisting of sequence SEQ ID NO: 33 or its complementary sequence, may consist of the sequence 5′-GAAGGTGACCAAGTTCATGCTACATTAATTTTCCAGCATTATTTCGACATG-3′ (SEQ ID NO: 34), the forward primer for detecting the resistant allele of the PE-0056550 marker, by amplifying a nucleic acid consisting of sequence SEQ ID NO: 33 or its complementary sequence, may consist of the sequence 5′-GAAGGTCGGAGTCAACGGATTGAACATTAATTTTCCAGCATTATTTCGACATA-3′ (SEQ ID NO: 35), and the common reverse primer may consist of the sequence 5′-TAGGTAATATTTGAGTTGCACTTATGGTAT-3′ (SEQ ID NO: 36). Using primers consisting of sequences SEQ ID NO: 34, SEQ ID NO: 35 and SEQ ID NO: 36, detection of a thymine (T) rather than a cytosine (C) at position 151 of the sequence SEQ ID NO: 33, or an alanine (A) rather than a guanine (G) in the complementary strand, indicates the presence of the QTL on chromosome 6 conferring resistance to powdery mildew (see Table 1 below).

In some embodiments, said QTL on chromosome 6 conferring resistance to powdery mildew is identified by detection of the PE-0021476 marker on chromosome 6. In particular, detection of the PE-0021476 marker on chromosome 6 is performed using two forward primers, one being specific for the resistant allele and one being specific for the susceptible allele, and one common reverse primer. Said two forward primers may be selected so as to enable amplifying a nucleic acid comprising or consisting of SEQ ID NO: 37, or a fragment thereof including the [G/C] polymorphism at position 51 of SEQ ID NO: 37. For example, the forward primer for detecting the susceptible allele of the PE-0021476 marker, by amplifying a nucleic acid consisting of sequence SEQ ID NO: 37 or its complementary sequence, may consist of the sequence 5′-GAAGGTCGGAGTCAACGGATTGTTGGGATTTTTAATTGACTGACTAAAC-3′ (SEQ ID NO: 38), the forward primer for detecting the resistant allele of the PE-0021476 marker, by amplifying a nucleic acid consisting of sequence SEQ ID NO: 37 or its complementary sequence, may consist of the sequence 5′-GAAGGTGACCAAGTTCATGCTTGTTGGGATTTTTAATTGACTGACTAAAG-3′ (SEQ ID NO: 39), and the common reverse primer may consist of the sequence 5′-CTCCAAAACCACAGCTACTAATTTGTT-3′ (SEQ ID NO: 40). Using primers consisting of sequences SEQ ID NO: 38, SEQ ID NO: 39 and SEQ ID NO: 40, detection of a cytosine (C) rather than a guanine (G) at position 51 of the sequence SEQ ID NO: 37, or a guanine (G) rather than a cytosine (C) in the complementary strand, indicates the presence of the QTL on chromosome 6 conferring resistance to powdery mildew (see Table 1 below).

The alleles conferring the resistances to powdery mildew amplified by the markers defined here above are as described in Table 1.

TABLE 1
Markers linked to resistance to powdery mildew on chromosome 6, location and flanking sequences. R: Resistant, S: Susceptible

| | | | | | | Alternative | |
|---|---|---|---|---|---|---|---|
| | Chromo- | Position | | Position in | | alleles | SEQ ID |
| Marker | some | (CM334 v.1.55) | Sequence surrounding the marker | SEQ ID NO | Type | (R/S) | NO |
| PE- | 6 | 232 015 212 | AAGATACTCTGCATTGGGTCCGGGCTAGAAGAATAAGCACGAATAACCGC | 51 | SNP | A/G | 1 |
| 0014628 | | | [A/G]TTCCAAGAAAATAGATTTGGTTGAGGAATTTGATCGAACACTTGGTG | | | | |
| | | | GGC | | | | |
| PE- | 6 | 233 673 276 | ATTTTCTAGGTTACTGGATTTAACTCAGGAGAAAATGGATTTGTTTTTGCAA | 151 | SNP | C/T | 5 |
| 0056542 | | | TGCATTTTTCCGGGGGTGCCTAGTTTATAAAGACCTAAGATGTTTCAGTCA | | | | |
| | | | ACTGCTTAATTAGGACCTTTGACTGTTGTGGTTATGACTGGGCCCCT[T/C] | | | | |
| | | | GCCAAGATGGTTTTGGACAACAGAGCTCTTACCACACTTGCTGCTATTTTG | | | | |
| | | | ATCTACTTGATCTTGAGTTGCGCTGTTTGCTGTGCTGTTCAAAGCGACATT | | | | |
| | | | GACTGTCTGAGATCTATAAAAGATTCATTAGAAGATCCAAATAATACT | | | | |
| PE- | 6 | 233 673 121 | AGATGGTTTTGGACAACAGAGCTCTTACCACACTTGCTGCTATTTTGATCT | 151 | SNP | A/G | 9 |
| 0056543 | | | ACTTGATCTTGAGTTGCGCTGTTTGCTGTGCTGTTCAAAGCGACATTGACT | | | | |
| | | | GTCTGAGATCTATAAAAGATTCATTAGAAGATCCAAATAATACTTTGA[G/A] | | | | |
| | | | CTATACATGGAAGTTCGNATAATCTGACGGAAGGTTTCATTTGCAAGTTTG | | | | |
| | | | CTGGGATTCAGTGCTGGCATCCTGATGAAAATAAGGTTCTGAGTATCAGT | | | | |
| | | | CTTCCTGACATGGGTCTCAAGGGCAGGTTTCCACTTGGTCTTCGGAATTG | | | | |
| PE- | 6 | 233 672 530 | CTGTTCCTGCAGAGAGCTATGCCAATAATCCCGGACTGTGTGGAGAACCG | 151 | SNP | T/G | 13 |
| 0056544 | | | TTAGATCCTTGCCAGGGTACTTCTAAGAAACCCCCTACCGCAATCATTGTC | | | | |
| | | | GGTGCAGCTGTTGGTGGGGTAAGTTTAGGTGCTGTATTTTTGGCCATCG | | | | |
| | | | [G/T]TATGTTCTTCTATATGAGAAGGATATCCAGGAAGAGGAAGAAGGATG | | | | |
| | | | ATGATCCTGAAGGGAATAAATGGGCGAAGAGTATTAAGGGTAAAAAGGCA | | | | |
| | | | ATCCAGGTAAGAATTTTAACTTCTCTGAATCATTAAAAACTGTCTCGTTACT | | | | |
| | | | G | | | | |
| PE- | 6 | 233 671 922 | TCTTTTCATAGAAGCATTTAACTAATGAATTTTCACCTGCTCCGCTGCTCG | 151 | SNP | C/T | 17 |
| 0056545 | | | TGTGTGATTTTTCTCTTGTTCAAGTTTTCTTAATTGACTTAAGGTTCCTTATT | | | | |
| | | | TTCAGCTTTCAATGTTTGAGAAGTCTACTTCAAAAATGAGATTTAG[T/C]GA | | | | |
| | | | TCTTATGAAGGCCACTGACAGCTTCAACAAAAACAATATTATCGGGTCAG | | | | |
| | | | GAAGAACCGGGACTTTTTACAAAGCAGTACTTGATGATGGCACTTCACTTA | | | | |
| | | | TGGTTAAGAGGTTGCAGAATACTCAGCACTCAGAGAAAGAGTTTATG | | | | |
| PE- | 6 | 233 671 648 | ACTCAGCACTCAGAGAAAGAGTTTATGTCCGAGATGGCTACATTGGGAAA | 151 | SNP | C/A | 21 |
| 0056546 | | | TGTAAAGCATCGTAATCTGGTTCCTCTTTTAGGTTTCTGCATTGCCAAAAA | | | | |
| | | | AGAAAGGCTGTTGGTCTACAAAGACATGCCAAATGGAACCTTGCATGAT | | | | |
| | | | [A/C]GGTTACATTCGGTGAGTGAAGGCGAAAAAACTCTAGAGTGGCCTATAA | | | | |
| | | | GATTAAAAATAGGCATTGGAGCAGCCAAAGGATTTGCATGGCTTCACCAC | | | | |
| | | | AACTGCAATCCTCGTATCATTCACAGAAATATTAGTTCTAAATGCATCTTG | | | | |
| PE- | 6 | 233 671 204 | GCTACTCCAAAAGGTGATGTGTACAGTTTCGGTGTTGTACTTCTCGAGTTA | 151 | SNP | C/A | 25 |
| 0056547 | | | GTTACTGGTGAGAAACCGACCTCTGTCACCAAAGCTCCCGAGACCTTTAA | | | | |
| | | | GGGGAATTTGGTGGAATGGATCACACAACTCTCCGGTGAATCTAAGCTT | | | | |
| | | | [A/C]AAGATGCGATTGACCATTCATTGTCTGGTAAAGGTTATGACAATGAGAT | | | | |
| | | | CTTCCAGGTCCTTAAAGTTGCTTGTCGATGTGTGTTGTCTCCTGTTCCTAA | | | | |
| | | | GGAGAGGCCGACAATGTTTGAAGTTTACCAGCTTCTGAGAGCCATCGGA | | | | |
| | | | G | | | | |
| PE- | 6 | 233 493 124 | TAACTGATGGTTTCAGTTGAGCTGCAAAATGGAACATCAATATCAGTAGCT | 151 | SNP | A/T | 29 |
| 0056549 | | | TCTCGTTTCAACCATTTACTTCCTTTGCTCTTTAGTCACTTGCACTTTAGAG | | | | |
| | | | AAGAATGACGTGATTCCCGATATTTTATACTTAGCTATTCTAGCATA[T/A]G | | | | |
| | | | GGAAACAAAACAGATGAATCATGAAACTCTTCGGTAGTAAAAAAAATTATG | | | | |
| | | | TGCCAAAATGTTGGATGTATCTTTTTATGGACGATGATCCATACCATGTTC | | | | |
| | | | GGATTCAGGATCCACTTTTACGATTGCATCCTCCCAGCTCTTCCATT | | | | |
| PE- | 6 | 233 489 782 | TTTGATTTTGTTTAACTCATTTGCATGAAATGATATGTTTATATGTTTGTCTA | 151 | SNP | T/C | 33 |
| 0056550 | | | GAAATATCACAACTATTACATTTATAGGTAATATTTGAGTTGCACTTATGGT | | | | |
| | | | ATAGTGCATTTTTATTTCTATTTATGTGACATTATTTTTTAGATA[C/T]ATGTC | | | | |
| | | | GAAATAATGCTGGAAAATTAATGTTCAACTCTTTGAATACATGAAAGAACC | | | | |
| | | | GAAACTTCTACCCTATTTTTAAGGGATGGACGCGTGAATTAGACCTAATTA | | | | |
| | | | AGAAATATTTATCTTACACTTTTTGTTTTATCGAGAAGCTTTT | | | | |
| PE- | 6 | 235 235 179 | CTCCAAAACCACAGCTACTAATTTGTTAGATCCAAAACTTAAAAGCTTAT | 51 | SNP | C/G | 37 |
| 0021476 | | | [C/G]TTTAGTCAGTCAATTAAAAATCCCAACAATTTAAAAGCTAAGAAGTGAAG | | | | |

Accordingly, in some embodiments, the presence of the QTL on chromosome 6 conferring resistance to Leveillula taurica is characterized by the presence of:
allele A of PE-0014628 (SEQ ID NO: 1);
allele C of PE-0056542 (SEQ ID NO: 5);
allele A of PE-0056543 (SEQ ID NO: 9);
allele T of PE-0056544 (SEQ ID NO: 13);
allele C of PE-0056545 (SEQ ID NO: 17);
allele C of PE-0056546 (SEQ ID NO: 21);
allele C of PE-0056547 (SEQ ID NO: 25);
allele A of PE-0056549 (SEQ ID NO: 29);
allele T of PE-0056550 (SEQ ID NO: 33); and/or
allele C of PE-0021476 (SEQ ID NO: 37).

A plant of the invention which comprises said QTL on chromosome 6 preferably comprises any combination of the alleles as defined here above.

In particular, said QTL on chromosome 6 conferring said resistance to powdery mildew is characterized by at least one of the following allele combinations:
Allele combination “A” comprising allele A of PE-0056543 (SEQ ID NO: 9), allele T of PE-0056544 (SEQ ID NO: 13), allele C of PE-0056545 (SEQ ID NO: 17), and allele C of PE-0056546 (SEQ ID NO: 21);
Allele combination “B” comprising allele C of PE-0056542 (SEQ ID NO: 5), allele A of PE-0056543 (SEQ ID NO: 9), allele T of PE-0056544 (SEQ ID NO: 13), allele C of PE-0056545 (SEQ ID NO: 17), allele C of PE-0056546 (SEQ ID NO: 21) and allele C of PE-0056547 (SEQ ID NO: 25);
Allele combination “C” comprising allele C of PE-0056542 (SEQ ID NO: 5), allele A of PE-0056543 (SEQ ID NO: 9), allele T of PE-0056544 (SEQ ID NO: 13), allele C of PE-0056545 (SEQ ID NO: 17), allele C of PE-0056546 (SEQ ID NO: 21), allele C of PE-0056547 (SEQ ID NO: 25), allele A of PE-0056549 (SEQ ID NO: 29), and allele T of PE-0056550 (SEQ ID NO: 33);
Allele combination “D” comprising allele A of PE-0014628 (SEQ ID NO: 1), allele C of PE-0056542 (SEQ ID NO: 5), allele A of PE-0056543 (SEQ ID NO: 9), allele T of PE-0056544 (SEQ ID NO: 13), allele C of PE-0056545 (SEQ ID NO: 17), allele C of PE-0056546 (SEQ ID NO: 21), allele C of PE-0056547 (SEQ ID NO: 25), allele A of PE-0056549 (SEQ ID NO: 29), allele T of PE-0056550 (SEQ ID NO: 33), and allele C of PE-0021476 (SEQ ID NO: 37); and
Allele combination “E” comprising allele A of PE-0056549 (SEQ ID NO: 29) and allele T of PE-0056550 (SEQ ID NO:33).

In some embodiments, said QTL on chromosome 6 conferring said resistance to powdery mildew is characterized by one of the allele combinations “A”, “B”, “C” or “D”, in particular by allele combination “A”. Alternatively or in addition, said QTL on chromosome 6 conferring said resistance to powdery mildew is characterized by allele combination “E”.

Alternatively or in addition, said QTL is characterized by the presence of at least one of:
allele A of PE-0006634 (SEQ ID NO: 57)
allele A of PE-0006718 (SEQ ID NO: 58)
allele G of PE-0012892 (SEQ ID NO: 59)
allele A of PE-0004191 (SEQ ID NO: 60)
allele A of PE-0005575 (SEQ ID NO: 61)
allele A of PE-0006320 (SEQ ID NO: 62)
allele T of PE-0016499 (SEQ ID NO: 63)
allele A of PE-0013625 (SEQ ID NO: 64); and/or
allele A of PE-0022671 (SEQ ID NO: 65)

Said QTL on chromosome 6 can comprise at least one of said alleles, in particular more than one, such as 2, 3, 4, 5, 6, 7, 8 or 9 of said alleles, or all of said alleles.

In a preferred embodiment, said QTL is characterized by the presence of
allele A of PE-0006718 (SEQ ID NO: 58), and/or
allele G of PE-0012892 (SEQ ID NO: 59)

In one embodiment said QTL on chromosome 6 confers a resistance to powdery mildew, in particular a moderate to high resistance to powdery mildew, in a plant which does not comprise the QTL of resistance to Leveillula taurica on chromosome 4 as described in International patent publication WO2013/033210 and/or which does not comprise the QTL of resistance to Leveillula taurica on linkage group LG1/8 as described in International Patent publication WO2014/140331.

In one embodiment, the plant of the invention does not comprise the QTL of resistance to Leveillula taurica on chromosome 4 as described in International patent publication WO2013/033210. Alternatively or in addition, the plant according to the invention does not comprise the QTL of resistance to Leveillula taurica on linkage group LG1/8 as described in International Patent publication WO2014/140331.

The inventors have identified two genes CA06g26150 and CA06g26250, located within the QTL on chromosome 6 conferring resistance to powdery mildew. Said genes have been identified as candidate genes which may confer the resistance to powdery mildew.

In some embodiments, said QTL on chromosome 6 co-segregates with the gene CA06g26150. CA06g26150 encodes a MLO-like protein. CA06g26150 is located between positions 233490196 and 233495756 of chromosome 6 of pepper genome Capsicum annuum cv. CM334 v.1.55.

In some embodiments, said resistance to powdery mildew is linked to the CA06g26150 gene. In particular, said resistance to powdery mildew is caused by at least one mutation within the CA06g26150 gene, in comparison with the CA06g26150 gene of a susceptible Capsicum plant and/or line. A coding sequence of the CA06g26150 gene of a susceptible plant (Maor) is set forth in SEQ ID NO: 41. The mutation may consist in at least one nucleotide substitution, insertion or deletion in the sequence of the CA06g26150 gene, in particular its coding sequence as set forth in SEQ ID NO: 41, including the deletion of the full gene or a fragment thereof. Preferably, the mutation is a loss-of-function mutation. The mutation may induce one or more amino acid substitutions in the sequence of the MLO-like protein encoded by the CA06g26150 gene, and impair the function of the MLO-like protein. In one embodiment, the loss-of-function mutation in the CA06g26150 gene is a null mutation. A null mutation prevents expression of an active MLO-like protein, for instance by causing a premature stop in the translation of the mRNA into a protein, resulting into the expression of a truncated form of the MLO-like protein. In one embodiment, the CA06g26150 gene of a Capsicum plant according to the invention comprises allele A of marker PE-0056549 (SEQ ID NO: 29).

In one embodiment, the present invention thus relates to a Capsicum plant resistant to powdery mildew, wherein said plant comprises a mutant allele of the CA06g26150 gene encoding a MLO-like protein, wherein said mutant allele confers said resistance.

In some embodiments, said QTL on chromosome 6 co-segregates with the gene CA06g26250. CA06g26250 encodes a leucine-rich repeat receptor-like protein kinase. CA06g26250 is located between positions 233670962 and 233673290 of chromosome 6 of pepper genome Capsicum annuum cv. CM334 v.1.55.

In some embodiments, said resistance to powdery mildew is linked to the CA06g26250 gene. In particular, said resistance to powdery mildew is caused by at least one mutation within the CA06g26250 gene, in comparison with the CA06g26250 gene of a susceptible Capsicum plant and/or line. A coding sequence of the CA06g26250 gene of a susceptible plant (Maor) is set forth in SEQ ID NO: 42. The mutation may consist in at least one nucleotide substitution, insertion or deletion in the sequence of the CA06g26250 gene, in particular its coding sequence as set forth in SEQ ID NO: 42, including the deletion of the full gene or a fragment thereof. Preferably, the mutation is a loss-of-function mutation. The mutation may induce one or more amino acid substitutions in the sequence of the leucine-rich repeat receptor-like protein kinase encoded by the CA06g26250 gene, and impair the function of the leucine-rich repeat receptor-like protein kinase. In one embodiment, the loss-of-function mutation in the CA06g26250 gene is a null mutation. A null mutation prevents expression of an active leucine-rich repeat receptor-like protein kinase, for instance by causing a premature stop in the translation of the mRNA into a protein, resulting into the expression of a truncated form of the leucine-rich repeat receptor-like protein kinase.

In one embodiment, the CA06g26250 gene of a Capsicum plant according to the invention comprises one or more of allele C of PE-0056542 (SEQ ID NO: 5), allele A of PE-0056543 (SEQ ID NO: 9), allele T of PE-0056544 (SEQ ID NO: 13), allele C of PE-0056545 (SEQ ID NO: 17), allele C of PE-0056546 (SEQ ID NO: 21) and allele C of PE-0056547 (SEQ ID NO: 25), in particular one or more of allele A of PE-0056543 (SEQ ID NO: 9), allele T of PE-0056544 (SEQ ID NO: 13), allele C of PE-0056545 (SEQ ID NO: 17) and allele C of PE-0056546 (SEQ ID NO: 21).

In one embodiment, the present invention thus relates to a Capsicum plant resistant to powdery mildew caused by the fungal pathogen Leveillula taurica, wherein said plant comprises a mutant allele of the CA06g26250 gene encoding a leucine-rich repeat receptor-like protein kinase, wherein said mutant allele confers said resistance.

In some embodiments, said QTL on chromosome 6 co-segregates with the gene CA06g26150 and/or the gene CA06g26250.

Genes CA06g26150 and CA06g26250 are annotated in the pepper genome Capsicum annuum cv. CM334 v.1.55. CA06g26150 and CA06g26250 correspond, respectively to LOC107874591 and LOC107875376, as annotated in the pepper genome Capsicum annuum cv. Zunla v 1.0.

In some embodiments, said Capsicum plant according to the invention is a plant of C. annuum line ST17-1157 which seeds are deposited at NCIMB accession number NCIMB 43602.

In some embodiments, a plant according to the invention may be a progeny or offspring of a plant grown from the deposited seeds of C. annuum line ST17-1157, deposited at the NCIMB under the accession number NCIMB 43602. Plants grown from the deposited seeds are homozygously resistant to powdery mildew. They can be used to transfer said QTL on chromosome 6 in another background by crossing and selfing and/or backcrossing. A progeny of a plant obtained from the deposited seed can be identified by one skilled in the art, for example by using one or more of the markers PE-0014628 (SEQ ID NO: 1), PE-0056542 (SEQ ID NO: 5), PE-0056543 (SEQ ID NO: 9), PE-0056544 (SEQ ID NO: 13), PE-0056545 (SEQ ID NO: 17), PE-0056546 (SEQ ID NO: 21), PE-0056547 (SEQ ID NO: 25), PE-0056549 (SEQ ID NO: 29), PE-0056550 (SEQ ID NO: 33) and PE-0021476 (SEQ ID NO: 37). Preferably, such a progeny is identified by at least 2, in particular at least 3 of said markers. Such progeny can also be identified by using one or more of the markers PE-0006634 (SEQ ID NO: 57), PE-0006718 (SEQ ID NO: 58), PE-0012892 (SEQ ID NO: 59), PE-0004191 (SEQ ID NO: 60), PE-0005575 (SEQ ID NO: 61), PE-0006320 (SEQ ID NO: 62), PE-0016499 (SEQ ID NO: 63), PE-0013625 (SEQ ID NO: 64), and PE-0022671 (SEQ ID NO: 65), in particular markers PE-0006718 (SEQ ID NO: 58) and/or PE-0012892 (SEQ ID NO: 59). Sequences and alleles for these markers are provided in the present specification.

The resistance to powdery mildew is advantageously determined by comparison to a susceptible line, preferably a susceptible commercial line, for example one or more of the Maor, CPNT, Vania or BYP lines, in particular from the Maor line. The resistance is preferably determined as detailed in example 2, on the basis of an inoculation test of the plant. The test can be a test on plantlets, or a test on an adult plant.

In one embodiment, the plant according to the invention shows a sporulation covering less than 10% of the leaves surface, preferably less than 5% of the leaves surface, still preferably no sporulation on the leaves in conditions of infection by Leveillula taurica, particularly in the same environmental conditions in which a susceptible Capsicum plant shows a sporulation covering more than 60% of the leave surfaces, preferably more than 70% of the leaves surface, still preferably more than 75% of the leaves surface. The sporulation rate can be assessed on the entire plant or on only a part of the plant, e.g. the bottom part or the medium part of the plant. The susceptible Capsicum plant may be a susceptible (commercial) line, for example one or more of the Maor, CPNT, Vania or BYP lines, in particular from the Maor line. In one embodiment, these properties are assessed on a population of at least 5, in particular at least 10, more particularly at least 50, even more particularly at least 100 plants.

The Capsicum plant according to the invention can be from any species within the Capsicum genus. In particular, it may be a Capsicum annuum, Capsicum baccatum, Capsicum frutescens, Capsicum chinense, Capsicum pubescens or Capsicum chacoense plant. Preferably, the plant according to the invention is a Capsicum annuum plant, more preferably a sweet pepper or a hot pepper, even more preferably a bell pepper. The Capsicum plant can also be from any type. In particular, the Capsicum plant may be of one of the following types: Dulce Italiano, Lamuyo, Blocky Florida, Open field and blocky protected, Ancho, Anaheim, Marconi, Jalapeno, Cayenne, Charleston or Sivri.

In one embodiment, the QTL on chromosome 6 according to the invention provides resistance to more than one strains of Leveillula taurica.

The invention also relates to a population of Capsicum plants according to the invention, wherein said population comprises at least 5 plants, in particular at least 10 plants, more particularly at least 20 plants, even more particularly at least 50 or 100, or more particularly at least 1000 plants.

According to a second aspect, the present invention is directed to one or more parts of a plant according to the invention.

All the embodiments detailed in the preceding section in connection with the first aspect of the invention are also embodiments according to this second aspect of the invention. In particular, the different features of said QTL that have been defined in relation with the above aspects of the invention apply mutatis mutandis to this aspect of the invention.

In some embodiments, said QTL is present on chromosome 6 in the genome of a seed of Capsicum annuum ST17-1157, deposited at NCIMB accession number NCIMB 43602.

In some embodiments, a plant part according to the invention comprises a QTL conferring resistance to powdery mildew is located on chromosome 6 within the chromosomal region delimited by PE-0014628 (SEQ ID NO: 1) and PE-0021476 (SEQ ID NO: 37).

In some embodiments, a plant part according to the invention comprises one or more alleles conferring resistance to powdery mildew as described in Table 1 and/or Table 12.

In some embodiments, the plant part according to the invention comprises any one of the allele combinations “A” to “E” as defined in the first aspect of the invention.

In some embodiments, the combination of alleles as described here above is as found in the genome of a plant corresponding to the deposited material ST17-1157 (NCIMB accession number NCIMB 43602).

According to another embodiment, the plant part is an embryo, protoplast, meristematic cell, callus, pollen, leaf, anther, stem, petiole, root, root tip, fruit, seed, flower, cotyledon, and/or hypocotyl.

In some embodiments, a part of a plant is a plant cell. The invention thus provides a cell of a Capsicum plant according to the invention, i.e. a plant cell that comprises comprising introgressed in its genome a quantitative trait locus (QTL) on chromosome 6, conferring resistance to powdery mildew, wherein said QTL is present on chromosome 6 and is as defined in the present specification. The different features of said QTL that have been defined in relation with the above aspects of the invention apply mutatis mutandis to this aspect of the invention.

A plant cell of the invention may have the capacity to be regenerated into a whole plant.

Alternatively, the invention is also directed to plant cells which are not regenerable, and thus are not capable of giving rise to a whole plant.

Preferably, a plant cell according to the invention is derived from an embryo, protoplast, meristematic cell, callus, pollen, leaves, anther, stem, petiole, root, root tip, fruit, seed, flower, cotyledon, or hypocotyl.

Preferably, a plant cell according to the invention comprises a QTL conferring resistance to powdery mildew is located on chromosome 6 within the chromosomal region delimited by PE-0014628 (SEQ ID NO: 1) and PE-0021476 (SEQ ID NO: 37).

The invention is more particularly concerned with a seed of a Capsicum plant, giving rise when grown up to Capsicum plant resistant to powdery mildew as defined above. Such seed are thus ‘seed of a plant of the invention’, i.e. seed giving rise to a plant of the invention. The invention is also concerned with seed from a plant of the invention, i.e. obtained from such a plant after selfing or crossing, provided however that the plant obtained from said seed is resistant to powdery mildew due to the QTL on chromosome 6 as defined here above conferring said resistance.

The invention also relates to a population of Capsicum seeds according to the invention, wherein said population comprises at least 2 seeds, especially at least 10 seeds, particularly at least 100 seeds, even more particularly at least 1000 seeds.

The present invention is also directed to a tissue culture of regenerable cells of the plant as defined above according invention, and thus comprises in their genome the QTL on chromosome 6 conferring resistance to powdery mildew as described here above.

The tissue culture will preferably be capable of regenerating plants having the physiological and morphological charac [symbol] invention.

The invention also provides a protoplast of the plant defined above, or from the tissue culture defined above, said proto

[symbol]

All the embodiments detailed above in connection with the first aspect of the invention are also embodiments according

[symbol]

According to another aspect, the present invention is also directed to the use of a Capsicum plant as detailed according to the first aspect of the invention, resistant to powdery mildew, as a breeding partner in a breeding program for obtaining Capsicum plants resistant to powdery mildew. Indeed, such a Capsicum plant according to the first aspect harbors in its genome the QTL as defined here above conferring said resistance. By crossing this plant with susceptible or less resistant plants, it is thus possible to transfer this QTL, conferring the desired phenotype, to the progeny. A plant according to the invention can thus be used as a breeding partner for introgressing the QTL conferring the desired phenotype into a Capsicum plant or germplasm. The invention is also directed to the same use with plants or seeds of ST17-1157 as deposited at NCIMB under accession number NCIMB 43602. Said plants are also suitable as introgression partners in a breeding program aiming at conferring the desired phenotype to a Capsicum plant or germplasm.

In such a breeding program, the selection of the progeny displaying the desired phenotype, or bearing sequences linked to the desired phenotype, can advantageously be carried out on the basis of the allele of the markers disclosed here above. The progeny is preferably selected on the presence of one or more of the following specific alleles: allele A of PE-0014628 (SEQ ID NO: 1); allele C of PE-0056542 (SEQ ID NO: 5); allele A of PE-0056543 (SEQ ID NO: 9); allele T of PE-0056544 (SEQ ID NO: 13); allele C of PE-0056545 (SEQ ID NO: 17); allele C of PE-0056546 (SEQ ID NO: 21); allele C of PE-0056547 (SEQ ID NO: 25); allele A of PE-0056549 (SEQ ID NO: 29); allele T of PE-0056550 (SEQ ID NO: 33); and/or allele C of PE-0021476 (SEQ ID NO: 37). The progeny is preferably selected on the presence of one or more of the allele combinations “A” to “E” as defined in the first aspect of the invention. The progeny may also be selected based on the presence of one or more of allele A of PE-0006634 (SEQ ID NO: 57), allele A of PE-0006718 (SEQ ID NO: 58), allele G of PE-0012892 (SEQ ID NO: 59), allele A of PE-0004191 (SEQ ID NO: 60), allele A of PE-0005575 (SEQ ID NO: 61), allele A of PE-0006320 (SEQ ID NO: 62), allele T of PE-0016499 (SEQ ID NO: 63), allele A of PE-0013625 (SEQ ID NO: 64) and/or allele A of PE-0022671 (SEQ ID NO: 65), in particular allele A of PE-0006718 (SEQ ID NO: 58) and/or allele G of PE-0012892 (SEQ ID NO: 59).

The selection of the progeny having the desired phenotype can also be made on conditions of a Leveillula taurica infection, as disclosed inter alia in Example 2, or with other tests well-known to the skilled reader.

A plant according to the invention, in particular a plant as deposited under accession number NCIMB 43602, is thus particularly valuable in a marker assisted selection program for obtaining commercial Capsicum lines and varieties resistant to powdery mildew.

The invention is also directed to the use of said plants in a program aiming at identifying, sequencing and/or cloning the genetic sequences conferring the desired phenotype.

Any specific embodiment described for the previous aspect of the invention is also applicable to this aspect of the invention, especially with regard to the features of the QTL conferring the phenotype of interest.

According to another aspect, the invention also concerns methods for the production of Capsicum plants resistant to powdery mildew, especially commercial plants. A method or process for the production of a plant having these features comprises the following steps:
(a) crossing a plant according to the first aspect of the invention (e.g. a plant corresponding to the deposited seeds (NCIMB 43602)) and a susceptible or less resistant Capsicum plant, in which the desired phenotype is to be imported or improved,
(b) selecting one plant resistant to powdery mildew in the progeny thus obtained, or one plant bearing the QTL on chromosome 6 as defined in the present invention conferring a resistance to powdery mildew,
(c) optionally self-pollinating one or several times the resistant plant obtained at step b) and selecting a plant resistant to powdery mildew,
(d) backcrossing the resistant plant selected in step b) or c) with a Capsicum plant susceptible to powdery mildew
(e) selecting a plant resistant to powdery mildew.

Alternatively, the method or process may comprise the following steps:
(a1) crossing a plant according to the first aspect of the invention (e.g. a plant corresponding to the deposited se
(a2) selfing the F1 population to create F2 population,
(b) selecting resistant individuals in the progeny thus obtained,
(c) optionally self-pollinating one or several times the resistant plant obtained at step b) and selecting a resistant plant in the progeny thus obtained,
(d) backcrossing the resistant plant selected in step b) or c) with a Capsicum plant susceptible to powdery mildew,
(e) selecting a plant resistant to powdery mildew.

In some embodiments, the plant resistant to powdery mildew caused by Leveillula taurica can be selected at steps b), c) and e).

The plant selected at step e) is preferably a commercial plant. In some embodiments, the selected plant is a bell pepper. In some embodiments, the selected plant is one of the following types Dulce Italiano, Lamuyo and blocky in China, Blocky Florida, Open field and blocky protected, Ancho, Anaheim, Marconi, Jalapeno, Cayenne, Charleston or Sivri.

Preferably, steps d) and e) are repeated at least twice and preferably three times, not necessarily with the same susceptible Capsicum plant. Said susceptible Capsicum plant is preferably a breeding line.

The self-pollination and backcrossing steps may be carried out in any order and can be intercalated, for example a backcross can be carried out before and after one or several self-pollinations, and self-pollinations can be envisaged before and after one or several backcrosses.

In some embodiments, such a method is advantageously carried out by using markers as described here above for one or more of the selections carried out at steps b), c) and/or e) for selecting plants resistant to powdery mildew.

In some embodiments, the markers for selecting plants resistant to powdery mildew are one or more of the markers PE-0014628 (SEQ ID NO: 1), PE-0056542 (SEQ ID NO: 5), PE-0056543 (SEQ ID NO: 9), PE-0056544 (SEQ ID NO: 13), PE-0056545 (SEQ ID NO: 17), PE-0056546 (SEQ ID NO: 21), PE-0056547 (SEQ ID NO: 25), PE-0056549 (SEQ ID NO: 29), PE-0056550 (SEQ ID NO: 33) and PE-0021476 (SEQ ID NO: 37). Further markers which can be used are one or more of PE-0006634 (SEQ ID NO: 57), PE-0006718 (SEQ ID NO: 58), PE-0012892 (SEQ ID NO: 59), PE-0004191 (SEQ ID NO: 60), PE-0005575 (SEQ ID NO: 61), PE-0006320 (SEQ ID NO: 62), PE-0016499 (SEQ ID NO: 63), PE-0013625 (SEQ ID NO: 64), and PE-0022671 (SEQ ID NO: 65), in particular markers PE-0006718 (SEQ ID NO: 58) and/or PE-0012892 (SEQ ID NO: 59).

In some embodiments, the plant selected at any one of steps b), c) and/or e) is preferably selected on the presence of one of the allele combinations “A” to “E” as defined in the first aspect of the invention.

The selection carried out at steps b), c) and/or e) can also be made using any type of genetic marker, in particular restriction fragment length polymorphisms (RFLPs), amplified fragment length polymorphisms (AFLPs), simple sequence repeats (SSRs), simple sequence length polymorphisms (SSLPs), single nucleotide polymorphisms (SNPs), insertion/deletion polymorphisms (Indels), variable number tandem repeats (VNTRs), and random amplified polymorphic DNA (RAPD), isozymes, and other markers known to those skilled in the art.

The selection of the progeny having the desired phenotype can also be made on conditions of disease infection, as disclosed inter alia in the Examples or with other tests well-known to the skilled reader.

The method used for allele detection can be based on any technique allowing the distinction between two different alleles of a marker, on a specific chromosome. Detection of a polymorphism can be made by electrophoretic techniques including a single strand conformational polymorphism (Orita, et al. (1989) Genomics, 8(2), 271-278), denaturing gradient gel electrophoresis (Myers (1985) EPO 0273085), or cleavage fragment length polymorphisms (Life Technologies, Inc., Gaithersburg, Md.), but the widespread availability of DNA sequencing often makes it easier to simply sequence amplified products directly. Once the polymorphic sequence difference is known, rapid assays for the detection of a polymorphism can be designed for progeny testing, generally involving some version of PCR amplification of specific alleles (PASA; Sommer, et al. (1992) Biotechniques 12(1), 82-87), or PCR amplification of multiple specific alleles (PAMSA; Dutton and Sommer (1991) Biotechniques, 11(6), 700-7002). In particular examples, PCR detection and quantification is carried out using two labeled fluorogenic oligonucleotide forward primers and an unlabeled common reverse primer, for example, KASPar™ (KBiosciences). Detection of a polymorphism can also be made by electrophoretic techniques including a single strand conformational polymorphism (Orita, et al. (1989) Genomics, 8(2), 271-278), denaturing gradient gel electrophoresis (Myers (1985) EPO 0273085), or cleavage fragment length polymorphisms (Life Technologies, Inc., Gaithersburg, Md.). The widespread availability of DNA sequencing often also enables to sequence amplified products directly.

The present invention also concerns a Capsicum plant obtained or obtainable by the methods described herein. Such a plant is indeed a Capsicum plant that is resistant to powdery mildew according to the first aspect of the invention.

According to a further aspect, the present invention is also directed to hybrid Capsicum plants obtainable by crossing a resistant plant according to the first aspect of the invention, such as a plant ST17-1157, a representative sample of seeds which have been deposited under the NCIMB accession number NCIMB 43602, or a resistant plant obtainable by the methods disclosed above, with a second Capsicum plant, for example a plant susceptible to powdery mildew infection, or a plant with a different, e.g. a lower, level of resistance to powdery mildew infection. A particularly preferred hybrid Capsicum plant, is a plant which displays any trait or phenotype of agronomical interest.

The invention is also directed to a method for obtaining commercial Capsicum plants that are resistant to powdery mildew, said method comprising the steps of:
backcrossing a plant obtained by germinating the deposited seeds ST17-1157 (NCIMB accession number NCIMB 43602) or a Capsicum plant according to the first aspect of the invention, with a Capsicum plant, for example a Capsicum plant susceptible to powdery mildew,
selecting a plant resistant to powdery mildew in the progeny thus obtained.

The selection in the second step is preferably carried out as detailed above for the other methods of the invention. Said selection is preferably carried out on the presence of one or more of the specific alleles of the markers for the QTL on chromosome 6 as described here above, as found in line ST17-1157.

The plant selected is preferably a commercial variety, cultivar or type of pepper. In some embodiments, the selected plant is a bell pepper. In some embodiments, the selected plant is one of the following types Dulce Italiano, Lamuyo and blocky in China, Blocky Florida, Open field and blocky protected, Ancho, Anaheim, Marconi, Jalapeno, Cayenne, Charleston or Sivri.

Also provided are methods for producing Capsicum plants seeds. In some embodiments, the methods comprise crossing the Capsicum plant according to the invention with itself or with another Capsicum plant, and harvesting the resultant seeds.

In addition to introgression of the QTL associated to resistance to powdery mildew, as detailed in the methods of the invention, said sequences can also be introduced into Capsicum background by genetic engineering in order to obtain a commercial Capsicum plant resistant to powdery mildew. The identification and cloning of the introgressed QTL from Capsicum conferring the desired phenotype, inter alia from the deposit, are routine for the skilled person.

It is noted that the seeds or plants of the invention may be obtained by different processes, in particular technical processes such as mutagenesis, e.g. chemical mutagenesis or UV mutagenesis, or genetic engineering such as guided recombination, and are not exclusively obtained by means of an essentially biological process.

According to such an aspect, the invention relates to a Capsicum plant or seed, preferably a non-naturally occurring Capsicum plant or seed, which may comprise one or more mutations in its genome, which provides the mutant plant a resistance to powdery mildew, which mutation is as present, for example, in the genome of plants of which a representative sample was deposited with the NCIMB under deposit number NCIMB 43602.

The mutations can have a natural cause (spontaneous mutations) or can be induced via methods such as mutagenesis. Mutagenesis methods are known in the art and include chemical mutagenesis using ethyl methanesulfonate (EMS). Other chemical mutagenic agents include but are not limited to, diethyl sufate (des), ethyleneimine (ei), propane sultone, N-methyl-N-nitrosourethane (mnu), N-nitroso-N-methylurea (NMU), N-ethyl-N-nitrosourea (enu), and sodium azide.

Alternatively, the mutations can be induced by means of irradiation, which is for example selected from x-rays, fast neutrons, UV radiation.

Mutagenesis techniques can be followed by an identification method such as TILLING. TILLING (Targeting Induced Local Lesions IN Genomes) is a general reverse genetics technique that uses traditional chemical mutagenesis methods to create libraries of mutagenized individuals that are later subjected to high throughput screens for the discovery of mutations. TILLING combines chemical mutagenesis with mutation screens of pooled PCR products, resulting in the isolation of missense and non-sense mutant alleles of the targeted genes. Thus, TILLING uses traditional chemical mutagenesis (e.g. EMS or MNU mutagenesis) or other mutagenesis methods (e.g. radiation such as UV) followed by high-throughput screening for mutations in specific target genes, such as cl, wt and/or y according to the invention. S1 nucleases, such as CEL1 or ENDO1, are used to cleave heteroduplexes of mutant and wild type target DNA and detection of cleavage products using e.g. electrophoresis such as a LI-COR gel analyzer system, see e.g. Henikoff et al. Plant Physiology 2004, 135: 630-636. TILLING has been applied in many plant species, including pepper (Kang, H. S., Kim, S. H., Lee, S. W. et al. Hortic. Environ. Biotechnol. (2018) 59: 447). Also EcoTILLING, whereby mutants in natural populations are detected, has been widely used, see Till et al. 2006 (Nat Protoc 1: 2465-77) and Comai et al. 2004 (Plant J 37: 778-86).

Preferably, the mutations are the integration of one QTL conferring resistance to powdery mildew, wherein said at least one QTL is present on chromosome 6, in replacement of the homologous sequences of a Capsicum plant. Even more preferably, the mutation is the substitution of the sequence delimited by marker PE-0014628 (SEQ ID NO: 1) and marker PE-0021476 (SEQ ID NO: 37) on chromosome 6 of a Capsicum genome, or a fragment thereof, by the homologous sequence on chromosome 6 present in the genome of a plant of which a representative sample was deposited with the NCIMB under deposit number NCIMB 43602, wherein the sequences or fragments thereof confer when combined resistance to powdery mildew.

In an embodiment, the invention relates to a method for obtaining a Capsicum plant or seed carrying one or more mutations in its genome, which provides the plant with a resistance to powdery mildew. Such a method is illustrated in Example 12 and may comprise:
a) treating M0 seeds of a Capsicum plant to be modified with a mutagenic agent to obtain M1 seeds;
b) growing plants from the thus obtained M1 seeds to obtain M1 plants;
c) producing M2 seeds by self-fertilisation of M1 plants; and
d) optionally repeating step b) and c) n times to obtain M2+n seeds.

The M2+n seeds are grown into plants and submitted to Leveillula taurica infection. The surviving plants, or those with the milder symptoms of Leveillula taurica infection, are multiplied one or more further generations while continuing to be selected for their resistance to powdery mildew. In this method, the M1 seeds of step a) can be obtained via chemical mutagenesis such as EMS mutagenesis. Other chemical mutagenic agents include but are not limited to, diethyl sufate (des), ethyleneimine (ei), propane sultone, N-methyl-N-nitrosourethane (mnu), N-nitroso-N-methylurea (NMU), N-ethyl-N-nitrosourea (enu), and sodium azide. Alternatively, the mutations are induced by means of irradiation, which is for example selected from x-rays, fast neutrons, UV radiation.

In another embodiment of the invention, the mutations are induced by means of genetic engineering. Such mutations also include the integration of sequences conferring the resistance to powdery mildew, as well as the substitution of residing sequences by alternative sequences conferring the resistance to powdery mildew.

The genetic engineering means which can be used include the use of all such techniques called New Breeding Techniques which are various new technologies developed and/or used to create new characteristics in plants through genetic variation, the aim being targeted mutagenesis, targeted introduction of new genes or gene silencing (RdDM). Example of such new breeding techniques are targeted sequence changes facilitated thru the use of Zinc finger nuclease (ZFN) technology (ZFN-1, ZFN-2 and ZFN-3, see U.S. Pat. No. 9,145,565, incorporated by reference in its entirety), Oligonucleotide directed mutagenesis (ODM), Cisgenesis and intragenesis, RNA-dependent DNA methylation (RdDM, which does not necessarily change nucleotide sequence but can change the biological activity of the sequence), Grafting (on GM rootstock), Reverse breeding, Agro-infiltration (agro-infiltration “sensu stricto”, agro-inoculation, floral dip), Transcription Activator-Like Effector Nucleases (TALENs, see U.S. Pat. Nos. 8,586,363 and 9,181,535, incorporated by reference in their entireties), the CRISPR/Cas system (see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference), engineered meganuclease re-engineered homing endonucleases, DNA guided genome editing (Gao et al., Nature Biotechnology (2016), doi: 10.1038/nbt.3547, incorporated by reference in its entirety), and Synthetic 5 genomics). A major part of today's targeted genome editing, another designation for New Breeding Techniques, is the applications to induce a DNA double strand break (DSB) at a selected location in the genome where the modification is intended. Directed repair of the DSB allows for targeted genome editing. Such applications can be utilized to generate mutations (e.g., targeted mutations or precise native gene editing) as well as precise insertion of genes (e.g., cisgenes, intragenes, or transgenes). The applications leading to mutations are often identified as site-directed nuclease (SDN) technology, such as SDN1, SDN2 and SDN3. For SDN1, the outcome is a targeted, non-specific genetic deletion mutation: the position of the DNA DSB is precisely selected, but the DNA repair by the host cell is random and results in small nucleotide deletions, additions or substitutions. For SDN2, a SDN is used to generate a targeted DSB and a DNA repair template (a short DNA sequence identical to the targeted DSB DNA sequence except for one or a few nucleotide changes) is used to repair the DSB: this results in a targeted and predetermined point mutation in the desired gene of interest. As to the SDN3, the SDN is used along with a DNA repair template that contains new DNA sequence (e.g. gene). The outcome of the technology would be the integration of that DNA sequence into the plant genome. The most likely application illustrating the use of SDN3 would be the insertion of cisgenic, intragenic, or transgenic expression cassettes at a selected genome location. A complete description of each of these techniques can be found in the report made by the Joint Research Center (JRC) Institute for Prospective Technological Studies of the European Commission in 2011 and titled “New plant breeding techniques—State-of-the-art and prospects for commercial development”, which is incorporated by reference in its entirety.

The present invention also provides methods for detecting and/or selecting a Capsicum plant that is resistant to powdery mildew, wherein said method comprises the step of detecting the presence of at least one QTL conferring resistance to powdery mildew, wherein said at least one QTL is present on chromosome 6.

Preferably, said QTL conferring resistance to powdery mildew that is present on chromosome 6 is located within a chromosomal region that is delimited by marker PE-0014628 and PE-0021476. In some embodiments, said QTL that is present on chromosome 6 can be identified by amplifying any one of the following markers: PE-0014628 (SEQ ID NO: 1), PE-0056542 (SEQ ID NO: 5), PE-0056543 (SEQ ID NO: 9), PE-0056544 (SEQ ID NO: 13), PE-0056545 (SEQ ID NO: 17), PE-0056546 (SEQ ID NO: 21), PE-0056547 (SEQ ID NO: 25), PE-0056549 (SEQ ID NO: 29), PE-0056550 (SEQ ID NO: 33) and PE-0021476 (SEQ ID NO: 37); or any other markers within the chromosomal region delimited by markers PE-0014628 (SEQ ID NO: 1) and PE-0021476 (SEQ ID NO: 37). Additionally, or alternatively, said QTL that is present on chromosome 6 can be identified by amplifying any one of the following markers: PE-0006634 (SEQ ID NO: 57), PE-0006718 (SEQ ID NO: 58), PE-0012892 (SEQ ID NO: 59), PE-0004191 (SEQ ID NO: 60), PE-0005575 (SEQ ID NO: 61), PE-0006320 (SEQ ID NO: 62), PE-0016499 (SEQ ID NO: 63), PE-0013625 (SEQ ID NO: 64) and PE-0022671 (SEQ ID NO: 65), in particular markers PE-0006718 (SEQ ID NO: 58) and/or PE-0012892 (SEQ ID NO: 59).

In some embodiments, a plant is selected if any one of the allele combinations “A”” to “E”, as defined in the first aspect material sample of the plant to be selected.

In some embodiments, detection of the markers described in the application, in particular one or more of PE-0014628 (SEQ ID NO: 1), PE-0056542 (SEQ ID NO: 5), PE-0056543 (SEQ ID NO: 9), PE-0056544 (SEQ ID NO: 13), PE-0056545 (SEQ ID NO: 17), PE-0056546 (SEQ ID NO: 21), PE-0056547 (SEQ ID NO: 25), PE-0056549 (SEQ ID NO: 29), PE-0056550 (SEQ ID NO: 33) and PE-0021476 (SEQ ID NO: 37), or one or more of PE-0006634 (SEQ ID NO: 57), PE-0006718 (SEQ ID NO: 58), PE-0012892 (SEQ ID NO: 59), PE-0004191 (SEQ ID NO: 60), PE-0005575 (SEQ ID NO: 61), PE-0006320 (SEQ ID NO: 62), PE-0016499 (SEQ ID NO: 63), PE-0013625 (SEQ ID NO: 64) and PE-0022671 (SEQ ID NO: 65), is performed by amplification, e.g. by PCR, using, for each marker, one forward primer which can be used for amplifying the resistant allele, one forward primer which can be used for amplifying the susceptible allele and one common reverse primer, for example using the KASPar (KBiosciences) technology. In particular, the primers for amplifying each of said markers may have the sequences as described in the first aspect of the invention, and detailed in table 11.

In a preferred embodiment, the amplification is as described in the examples. In a still preferred embodiment, the amplification is performed using a two-step touchdown method in which the elongation and annealing steps are incorporated into a single step. The temperature used for the annealing stage determines the specificity of the reaction and hence the ability of the primers to anneal to the DNA template. A touchdown PCR involves a first step of Taq polymerase activation, followed by a second step called the touchdown step that involves a high annealing temperature and incrementally decreasing the annealing temperature in each PCR cycle, and a third step of DNA amplification. The higher annealing temperatures in the early cycles of a touchdown ensure that only very specific base pairing will occur between the DNA and the primer, hence the first sequence to be amplified is most likely to be the sequence of interest. The annealing temperature is gradually decreased to increase the efficiency of the reaction. The regions that were originally amplified during the highly specific early touchdown cycles will be further amplified and outcompete any non-specific amplification that may occur at the lower temperatures.

In another embodiment, the amplification of SNP markers is performed as recommended in the KASPar assay and illustrated in the examples, namely by PCR cycles, comprising a first denaturation step at 94° C. during around 15 minutes, at least 10 cycles of around 20 seconds at 94° C. followed by around 60 second at a decreasing temperature from 65° C. for the 1^{st} cycle to 57° C. for the last cycle, and around 35 cycles of around 20 seconds at 94° C. followed by around 60 seconds at 57° C. This protocol can easily be adapted by a skilled person, depending on the type of primers used.

According to a further aspect, the present invention also provides molecular markers that are linked to the QTL on chromosome 6 as defined here above conferring the resistance to powdery mildew.

In some embodiments, said molecular markers linked to the QTL conferring the resistance to powdery mildew on chromosome 6 are any one of the markers PE-0014628 (SEQ ID NO: 1), PE-0056542 (SEQ ID NO: 5), PE-0056543 (SEQ ID NO: 9), PE-0056544 (SEQ ID NO: 13), PE-0056545 (SEQ ID NO: 17), PE-0056546 (SEQ ID NO: 21), PE-0056547 (SEQ ID NO: 25), PE-0056549 (SEQ ID NO: 29), PE-0056550 (SEQ ID NO: 33) and PE-0021476 (SEQ ID NO: 37), or all the markers PE-0014628 (SEQ ID NO: 1), PE-0056542 (SEQ ID NO: 5), PE-0056543 (SEQ ID NO: 9), PE-0056544 (SEQ ID NO: 13), PE-0056545 (SEQ ID NO: 17), PE-0056546 (SEQ ID NO: 21), PE-0056547 (SEQ ID NO: 25), PE-0056549 (SEQ ID NO: 29), PE-0056550 (SEQ ID NO: 33) and PE-0021476 (SEQ ID NO: 37), or a combination of the markers PE-0014628 (SEQ ID NO: 1), PE-0056542 (SEQ ID NO: 5), PE-0056543 (SEQ ID NO: 9), PE-0056544 (SEQ ID NO: 13), PE-0056545 (SEQ ID NO: 17), PE-0056546 (SEQ ID NO: 21), PE-0056547 (SEQ ID NO: 25), PE-0056549 (SEQ ID NO: 29), PE-0056550 (SEQ ID NO: 33) and PE-0021476 (SEQ ID NO: 37) such as a combination corresponding to any one of allele combinations A to E, or any other markers within the chromosomal region delimited by marker PE-0014628 (SEQ ID NO: 1) and marker PE-0021476 (SEQ ID NO: 37). Alternatively, or in addition, said molecular markers linked to the QTL conferring the resistance to powdery mildew on chromosome 6 are any one of the markers PE-0006634 (SEQ ID NO: 57), PE-0006718 (SEQ ID NO: 58), PE-0012892 (SEQ ID NO: 59), PE-0004191 (SEQ ID NO: 60), PE-0005575 (SEQ ID NO: 61), PE-0006320 (SEQ ID NO: 62), PE-0016499 (SEQ ID NO: 63), PE-0013625 (SEQ ID NO: 64) and PE-0022671 (SEQ ID NO: 65).

The sequences of the markers as mentioned above are described in Table 1 above and Table 12 below.

Further provided is the use of one or more of the molecular markers PE-0014628 (SEQ ID NO: 1), PE-0056542 (SEQ ID NO: 5), PE-0056543 (SEQ ID NO: 9), PE-0056544 (SEQ ID NO: 13), PE-0056545 (SEQ ID NO: 17), PE-0056546 (SEQ ID NO: 21), PE-0056547 (SEQ ID NO: 25), PE-0056549 (SEQ ID NO: 29), PE-0056550 (SEQ ID NO: 33) and PE-0021476 (SEQ ID NO: 37) or all the markers PE-0014628 (SEQ ID NO: 1), PE-0056542 (SEQ ID NO: 5), PE-0056543 (SEQ ID NO: 9), PE-0056544 (SEQ ID NO: 13), PE-0056545 (SEQ ID NO: 17), PE-0056546 (SEQ ID NO: 21), PE-0056547 (SEQ ID NO: 25), PE-0056549 (SEQ ID NO: 29), PE-0056550 (SEQ ID NO: 33) and PE-0021476 (SEQ ID NO: 37), or a combination of the markers PE-0014628 (SEQ ID NO: 1), PE-0056542 (SEQ ID NO: 5), PE-0056543 (SEQ ID NO: 9), PE-0056544 (SEQ ID NO: 13), PE-0056545 (SEQ ID NO: 17), PE-0056546 (SEQ ID NO: 21), PE-0056547 (SEQ ID NO: 25), PE-0056549 (SEQ ID NO: 29), PE-0056550 (SEQ ID NO: 33) and PE-0021476 (SEQ ID NO: 37), or any other markers within the chromosomal region delimited by marker PE-0014628 (SEQ ID NO: 1) and marker PE-0021476 (SEQ ID NO: 37), for detecting a Capsicum plant that is resistant to powdery mildew.

The invention is also directed to the use of at least one of the markers PE-0014628 (SEQ ID NO: 1), PE-0056542 (SEQ ID NO: 5), PE-0056543 (SEQ ID NO: 9), PE-0056544 (SEQ ID NO: 13), PE-0056545 (SEQ ID NO: 17), PE-0056546 (SEQ ID NO: 21), PE-0056547 (SEQ ID NO: 25), PE-0056549 (SEQ ID NO: 29), PE-0056550 (SEQ ID NO: 33) and PE-0021476 (SEQ ID NO: 37) or all the markers PE-0014628 (SEQ ID NO: 1), PE-0056542 (SEQ ID NO: 5), PE-0056543 (SEQ ID NO: 9), PE-0056544 (SEQ ID NO: 13), PE-0056545 (SEQ ID NO: 17), PE-0056546 (SEQ ID NO: 21), PE-0056547 (SEQ ID NO: 25), PE-0056549 (SEQ ID NO: 29), PE-0056550 (SEQ ID NO: 33) and PE-0021476 (SEQ ID NO: 37), associated with a QTL on chromosome 6 conferring the resistance to powdery mildew according to the invention, for identifying alternative molecular markers associated with said QTLs, wherein said alternative molecular markers are in the chromosomal region delimited on chromosome 6 by marker PE-0014628 (SEQ ID NO: 1) and marker PE-0021476 (SEQ ID NO: 37).

The alternative molecular markers are preferably associated with said QTL with a p-value of 0.05 or less, preferably less than 0.01. The QTL is to be found in the deposited seeds NCIMB 43602.

The invention is also directed to a method for identifying a molecular marker associated with a QTL conferring resistance to powdery mildew when present heterozygously or homozygously, comprising:
(a) identifying a molecular marker in the chromosomal region delimited on chromosome 6 by marker PE-0014628 (SEQ ID NO: 1) and marker PE-0021476 (SEQ ID NO: 37),
(b) determining whether said molecular marker is associated with or linked to the resistance to resistance to powdery mildew in a segregating population issued from a plant exhibiting said resistance.

The population is preferably derived from a plant grown from the deposited seeds NCIMB 43602 or from a progeny thereof, exhibiting the resistance to powdery mildew as described in the invention.

In particular, the QTL on chromosome 6 mentioned above, conferring the resistance to powdery mildew according to the invention, is the QTL present on chromosome 6 in ST17-1157 (NCIMB 43602).

Genetic association or linkage is as defined above. Preferably the association or linkage is with a p-value of preferably less than 0.05, and most preferably less than 0.01 or even less.

A molecular marker and the resistance phenotype are inherited together in preferably more than 90% of the meiosis, preferably more than 95%.

In a further aspect, the invention relates to method for the production of Capsicum plantlets or plants resistant to powdery mildew, which method comprises:
i. culturing in vitro an isolated cell or tissue of the Capsicum plant according to the invention to produce Capsicum micro-plantlets resistant to powdery mildew, and
ii. optionally further subjecting the Capsicum micro-plantlets to an in vivo culture phase to develop into Capsicum plants resistant to powdery mildew.

The isolated cell or tissue used to produce a micro-plantlet is an explant obtained under sterile conditions from a Capsicum parent plant of the invention to be propagated. The explant comprises or consists, for instance, of a cotyledon, hypocotyl, stem tissue, leaf, embryo, meristem, node bud, shoot apice, or protoplast. The explant can be surface sterilized before being placed on a culture medium for micropropagation.

Conditions and culture media that can be suitably used in plant micropropagation are well known to those skilled in the art of plant cultivation and are described, for example, in “Plant Propagation by Tissue Culture, Handbook and Directory of Commercial Laboratories, eds. Edwin F George and Paul D Sherrington, Exegetics Ltd, 1984”.

Micropropagation typically involves:
1. axillary shoot production: axillary shoot proliferation is induced by adding cytokinin to the shoot culture medium, to produce shoots preferably with minimum callus formation;
ii. adventitious shoot production: addition of auxin to the medium induces root formation, in order to produce plantlets that are able to be transferred into the soil. Alternatively, root formation can be induced directly into the soil.

Plantlets can be further subjected an in vivo culture phase, by culture into the soil under lab conditions, and then progressive adaptation to natural climate, to develop into Capsicum plant resistant to powdery mildew.

In view of the ability of the resistant plants of the invention to restrict the damages caused by powdery mildew, they are advantageously grown in an environment infested or likely to be infested or infected by Leveillula taurica; in these conditions, the resistant plants of the invention produce more marketable peppers than susceptible plants. The invention is thus also directed to a method for improving the yield of Capsicum plants and/or fruits or for increasing the number of harvestable Capsicum fruits, in an environment infested by powdery mildew comprising growing in said environment Capsicum plants resistant to powdery mildew as defined, comprising on chromosome 6 the QTLs or sequences according to the invention and conferring to said plants resistance to powdery mildew. The invention is also directed to the use of the Capsicum plants of the invention for improving the yield of Capsicum plants and/or fruits, and/or for increasing the number of harvestable Capsicum fruits, in an environment infested by powdery mildew.

Preferably, the method comprises a first step of choosing or selecting a Capsicum plant comprising said sequences of interest conferring to said plants resistance to powdery mildew. The method can also be defined as a method of increasing the productivity of a Capsicum field, tunnel or glasshouse, or as a method of reducing the intensity or number of chemical or fungicide applications in the production of peppers.

The invention is also directed to a method for reducing the loss on Capsicum production in condition of Leveillula taurica infection, comprising growing a Capsicum plant as defined above.

The resistant plants of the invention are also able to restrict the growth of the pathogens responsible for powdery mildew, thus limiting the infection of further plants and the propagation of the pathogens. Accordingly, the invention is also directed to a method for protecting a field, tunnel or glasshouse, or any other type of plantation, from powdery mildew infection, or of at least limiting the level of infection or limiting the spread of powdery mildew. Such a method preferably comprises the step of growing a resistant or tolerant plant of the invention, i.e. a plant comprising on chromosome 6 the sequences conferring resistance to powdery mildew.

The invention also concerns the use of a Capsicum plant resistant to powdery mildew, according to the invention, in a field, tunnel or glasshouse, or other plantation. Preferably, the invention concerns the use of a Capsicum plant resistant to powdery mildew according to the invention for controlling infestation in a field, tunnel, glasshouse or any other plantation, by Leveillula taurica.

All the preferred features of the QTL are as defined in connection with the other aspects of the invention, in particular it is preferably present in the seeds of ST17-1157 (NCIMB accession number NCIMB 43602), and it is identifiable by the markers as defined according to the present invention.

The present invention is also directed to a method for improving the yield of Capsicum plants in an environment infested by powdery mildew comprising:
(a) identifying Capsicum plants resistant to powdery mildew comprising in their genome at least one QTL conferring resistance to powdery mildew, wherein said at least one QTL is present on chromosome 6, and
(b) growing said resistant Capsicum plants in said infested environment.

By this method, the yield of the Capsicum plants is increased, inter alia more marketable peppers can be harvested, or more commercial peppers are produced, or more seeds are obtained.

In still a further aspect, the invention also relates to a method of producing pepper fruits comprising:
(a) growing a Capsicum plant of the invention, as defined previously;
(b) allowing said plant to set fruit; and
(c) harvesting fruit of said plant, preferably at maturity and/or before maturity.

All the preferred embodiments regarding the Capsicum plant are already disclosed in the context of the previous aspects of the invention.

The method may advantageously comprise a further step of processing said peppers into a processed food.

The present invention also relates to a method of producing a food product, comprising mixing a pepper fruit of the invention, or part thereof, with one or more food ingredients. Optionally, the method comprises cooking and/or processing the pepper fruit of the invention, alone or in mixture with the one or more food ingredients. Examples of food products that comprise pepper in raw, cooked or otherwise processed form include powders, soups, sauces, salsas, pastas, condiments, pastries, sweets and salads.

The present invention also relates to a food product made of a pepper fruit of the invention or parts thereof, optionally in processed form.

In another aspect, the invention relates to the use of a Capsicum plant according to the invention or a fruit thereof in the fresh cut market or for food processing. Techniques for using pepper in food processing are well known from the skilled person, e.g. as an ingredient in a food product such as powders, soups, sauces, salsas, pastas, condiments, pastries, sweets and salads, and described, for instance, in Handbook of Food Science, Technology and Engineering, vol. 4, Y. H. Hui, Frank Sherkat. CRC Press.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

### Seed Deposit

A representative sample of seeds from the Capsicum plant according to the invention (i.e. seeds from Capsicum annuum ST17-1157 plant) has been deposited by HM-Clause, S. A., Rue Louis Saillant, Z. I. La Motte, BP83, 26802 Portes-lès-Valence cedex, France, pursuant to, and in satisfaction of, the requirements of the Budapest Treaty on the International Recognition of the Deposit of Microorganisms for the Purposes of Patent Procedure (the “Budapest Treaty”) with the National Collection of Industrial, Food and Marine Bacteria (NCIMB), 23 St Machar Drive, Aberdeen, Scotland, AB21 9YA, United Kingdom, on Apr. 22, 2020, under accession number NCIMB 43602.

A deposit of the ST17-1157 seeds is maintained by HM-Clause, S. A., Rue Louis Saillant, Z. I. La Motte, BP83, 26802 Portes-18s-Valence cedex, France.

### EXAMPLES

### Example 1: Identification of Resistance Source to Pepper Powdery Mildew

Several Capsicum annuum lines were grown in a greenhouse when a natural infection of Leveillula taurica started to build up. At the end of the growing cycle, certain plants from one of the lines, LT17, appeared to be symptomless while the entire greenhouse had Leveillula symptoms. Cultivar Maor, known as a susceptible control for Leveillula taurica was present on either side of the resistant plants and was fully infected by Leveillula taurica. The inventors thus decided to further evaluate the resistance level of LT17.

### Example 2: Evaluation of Resistance Level of the LT17 Line

Three seed lots of LT17 were evaluated according to an “artificial” test consisting in the artificial inoculation of the fungus Leveillula taurica on 4-6 leaf stage plantlets.

Artificial inoculation was conducted on several plantlets per lot. To prepare the inoculum, spores of Leveillula taurica were collected by rinsing leaves of sensible pepper varieties infected with the fungus, with distilled water. The spores were counted using a Nageotte counting cell (concentration of about 10⁴ c/ml). 1 mL/L Triton X 100 was added (Molot, Jp Leroux, M Diop-Bruckler. Agronomie, EDP Sciences, 1990, 10 (7), pp. 551-559. ffhal-00885316f.

The inoculum was sprayed on pepper plantlets at the 4-6 leaves stage. For each plantlet, the 4 oldest leaves were sprayed, on both faces. The inoculum was sprayed two times on each plantlet, one week apart. 4 weeks after the 1^{st} inoculation, the severity of the infection on plants was scored according to the following rating scale, based on percentages of infected area:

TABLE 2
scoring scale used in the artificial test

| | Percentage of | |
|---|---|---|
| Score | infected area | Descriptions |
| 9 | 0% | — |
| 8 | 0% | No sporulation but “small |
| | | spots” present |
| 7 | <10% | — |
| 6 | 10-25% | |
| 5 | 25-50% | non-infected leaves are lightly |
| | | affected only on the lower face |
| 3 | 50-75% | presence on the upper face of |
| | | the leaves |
| 1 | 75-100% | strong presence on both faces |

The following lines were used as control: Jaune Dupoux, Pipo, Piperade (susceptible); OCP32, OAP35, ODS200, ODS257 (intermediate) and HV12 (resistant).

The artificial test reveals that the three seed lots segregate for Leveillula taurica resistance. Specifically, plants with the highest level of resistance (9) are present within the three seed lots.

In order to confirm the level of resistance in tunnel and on adult plants, plants from artificial test with a resistance score of 9 were then transplanted in tunnel for a second stage of resistance evaluation.

In the same tunnel, three different lots of LT17, as well as plants from the Maor line and an F1 [Maor*LT17] were sown and planted. The sensitive plants were positioned at the entry and exit of the tunnel, as well as at regular intervals throughout the tunnel in order to ensure a homogenous and intense Leveillula pressure during the assay.

2 artificial inoculations (2 weeks interval) were carried out, by spraying the median part of the plants with inoculum with Leveillula taurica and evaluations were performed at the end of the test (8 weeks after the first inoculation). A specific rating scale for tunnel evaluation was used. Each plant was scored for three different notes: Note bot=percentage of infected leaves area in the bottom part of the plant (calculated as the mean of the 5 more infected leaves); Note med=percentage of infected leaves area in the medium part of the plant (calculated as the mean of the 5 more infected leaves)”; and Note Glo=percentage of infected leaves per plants (whatever the percentage of sporulation).

For each plant, an Index 1 (I1) was also calculated, corresponding to the mean of Note bot, Note med and Note glo.

Notes were attributed according to the following scale:

TABLE 3
Scoring scale for “Note bot” and “Note med” in the tunnel test
Scale for Note bot and Note med

| | Percentage | |
|---|---|---|
| | of plants with | |
| Note | mycelium | Descriptions |
| 9 | 0 | nothing |
| 8 | 0* | Very few spots of |
| | | penetration (and not |
| | | mycelium) |
| 7 | 1-10% | % of the infected leaves |
| 6 | 10-20% | area (Mean of the more |
| 5 | 20-30% | infected leaves) |
| 4 | 30-40% | |
| 3 | 40-50% | |
| 2 | 50-60% | |
| 1 | >60% | |

TABLE 4
Scoring scale for “Note glo” in the tunnel test
Scale for Note Glo

| | Percentage | |
|---|---|---|
| | of plants with | |
| Note | mycelium | Descriptions |
| 9 | 0% | Percentage of infected |
| 8 | 0.1-10% | leaves per plant |
| 7 | 10-20% | (whatever the percentage |
| 6 | 20-30% | of sporulation) |
| 5 | 30-40% | |
| 4 | 40-50% | |
| 3 | 50-60% | |
| 2 | 60-70% | |
| 1 | >70% | |

The plants were also tested for the presence of the RZ marker. This marker is disclosed in International Patent publication WO2014/140331 as a marker linked to a QTL of resistance to Leveillula taurica on linkage group LG1/8 of pepper genome. In the marker linked to QTL on LG1/8, the A in susceptible plants is replaced with a G in resistant plants. For detection of the RZ marker, the Kaspar technology was used. Plant DNA was amplified using the 3 primers CATGTGAGCGACTCCTCTGACAA (primer for the susceptible allele, SEQ ID NO: 43), ATGTGAGCGACTCCTCTGACAG (primer for the resistant allele, SEQ ID NO: 44) and CGAGGAGAATTTCCTGCTAAAATTGAGTT (Common primer, SEQ ID NO: 45).

TABLE 5
Resistance levels and RZ marker screening of
plants of the LT17 line in the tunnel test

| | | L. taurica Resistance |
|---|---|---|
| Plant | RZ marker | level |
| 1 | A:G | 9 |
| 2 | G:G | 9 |
| 3 | A:A | 7 |
| 4 | A:G | 9 |

Table 5 above shows the resistance levels for four plants of the LT17 line planted in tunnel. A high level of resistance to L. taurica is present in several plants of the LT17 line, which confirms the results obtained using the artificial test. The screening experiment on the RZ marker suggests that the QTL on linkage group LG1/8 is not responsible for the resistance.

Progenies of these plants obtained by selfing were tested for Leveillula taurica resistance in an artificial test, as described above. 2*10 plants were tested per progeny. The RZ marker was screened on individual plants.

TABLE 6
Resistance levels and RZ marker screening of progeny
plants of the LT17 line in the artificial test.

| | | L. taurica Resistance level |
|---|---|---|
| Plant number | RZ marker | in artificial test |
| 1_17LT05-1 | A:A | 9 |
| 1_17LT05-3 | A:A | 9 |
| 1_17LT05-4 | A:A | 9 |
| 1_17LT05-5 | A:A | 9 |
| 1_17LT05-6 | A:A | 9 |
| 1_17LT05-7 | A:A | 9 |
| 2_17LT05-9 | A:A | 9 |
| 2_17LT05-10 | A:A | 9 |
| 2_17LT06-1 | A:G | 9 |
| 2_17LT06-2 | A:G | 9 |
| 3_17LT06-8 | A:G | 9 |
| 5_17LT09-5 | G:G | 9 |
| 6_17LT09-6 | G:G | 9 |
| 7_17LT05-17 | A:A | 9 |
| 7_17LT05-20 | A:A | 9 |
| 8_17LT06-12 | A:A | 9 |
| 10_17LT08-12 | A:G | 9 |
| 10_17LT08-15 | A:G | 9 |

The results in Table 6 shows that resistant plants comprise a diversity of genotypes at the RZ markers, i.e. homozygo [symbol] is not implied in the resistance to powdery mildew as found in certain plants of the LT17 lines and their progeny.

These plants have been transplanted in tunnel for another Leveillula taurica resistance evaluation in autumn.

### Example 3: Genetic Analysis

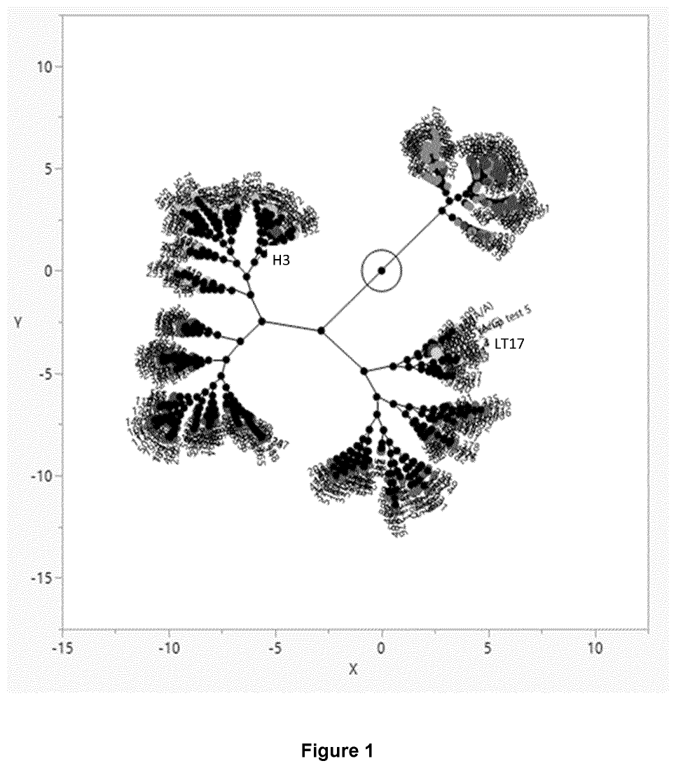

Genetic distance analysis of line LT17 was carried out on a high density Affymetrix SNP chip array, using 590 accessions and 5005 SNPs. The genetic distance tree in FIG. 1 shows that LT17 belongs to a Capsicum annuum cluster, but is clustered in a different group than line H3 (Daubeze, 1995; Lefebvre 2003). The source of the resistance, LT17 is thus genetically unrelated to the powdery mildew resistant lines H3 and its progeny HV12.

A plant from the LT17 line was also genotyped on a high density genotyping chip (Metachip), together with several plants from another source of Leveillula taurica resistance, the PBC167 line (International patent application WO2013/033210). About 36% of the 5360 analyzed SNPs exhibited a different allele between the LT17 line and the PBC167 line, showing that the two lines are genetically unrelated and distinct.

### Example 4: Development of F2 Resistance Segregating Population

A segregating F2 population from the cross between the susceptible line Maor and the LT17 line displaying resistance was developed to generate a linkage map and detect QTLs involved in the resistance (QTLs mapping).

179 F2 plants were genotyped with 2565 SNP markers evenly spaced along the full genome and being polymorphic between both Maor and LT17 parental lines.

The same 179 F2 plants as well as the 2 parental lines and the hybrid F1 Maor×LT17 were evaluated for Leveillula taurica resistance in tunnel under natural and artificial pressure create through two artificial inoculations performed with a Leveillula taurica strain (see Example 2)

The notation was made using the same three notes (Note bot, Note med and Note Glo) and scale than in Example 2 but was performed at 3 different times with 3 weeks interval for each notation (the first notation was done 2 weeks after the first inoculation).

This kinetic allows to calculate an AUDPC (Area under the disease progress curve) which is a powerful quantitative index to perform QTLs analysis. The AUDPC was calculated for each note and for the Index 1 (I1) by using the following formula:

$$AUDPC {\sum }_{i = 1}^{n - 1} ( \frac{{y}_{i} + {y}_{i + 1}}{2} ) ( {t}_{i + 1} - {t}_{i} )$$

“n” is the number of symptom assessments, “y” the symptom intensity and “t” the time in dpi (days post inoculation).

AUDPC was calculated plant by plant, for each note.

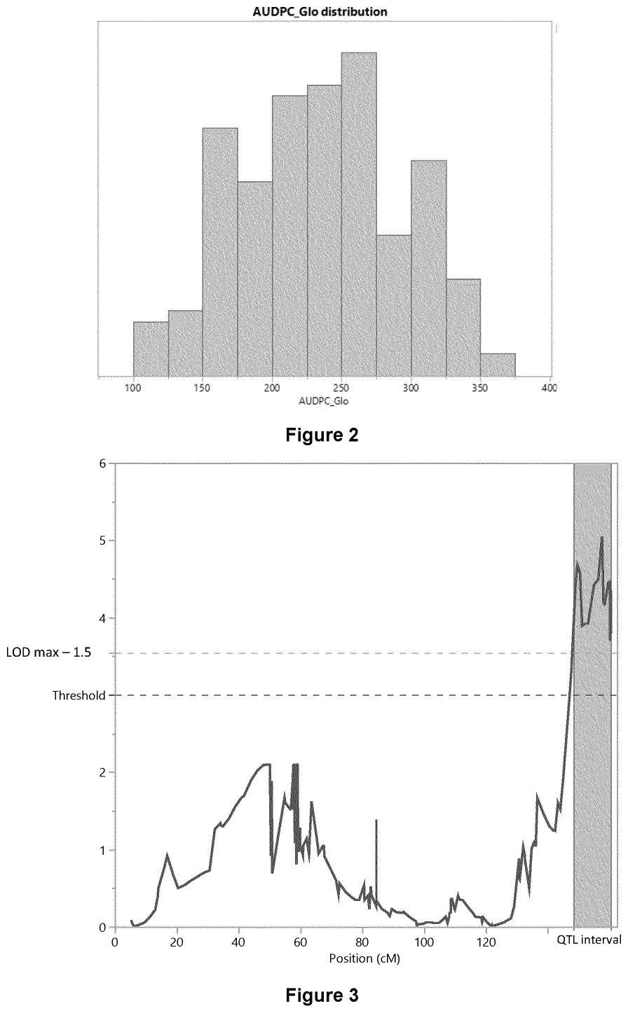

FIG. 2 presents the distribution of the “AUDPC Glo” in the F2 population. This figure shows that the segregation of the trait follows a “normal distribution” which highlights a complex inheritance of the trait. According to this segregation, these data were used to carry out QTL mapping analyses.

### Example 5: QTL Mapping Analyses in F2 Population Segregating Population

Segregation of each SNP within the F2 population was evaluated using the Chi-square test X². SNP markers exhibiting distortion of segregation tested against the Normal Mendelian expectation ratios −1:2:1 for an F2 were removed from the analyses.

Significance levels used is from P-value<0.01

A genetic linkage map was then performed with the JoinMap V4 software to assign genetic position of each SNP. Linkage groups were then assigned to chromosome using the physical position of the SNPs on a reference genome of Maor, version v3.0. QTL analyses (the association between genotypic variation and the trait variation) were performed with MapQTL V5 software.

We determined the number of genomic regions involved in the quantitative trait as well as their position and their effect, using the H0 hypothesis: no segregating QTL (locus with no genetic effect) and the alternative hypothesis H1: presence of a segregating QTL.

Putative QTLs were first detected using the Interval mapping module of the software (single-QTL model). A QTL likelihood map was determined: for each position on the genome—in our case each 2 cM—the likelihood for the presence of a segregating QTL was calculated.

Then the likelihood under H0 and H1 hypothesis were compared with a likelihood ratio statistic (LOD score): A QTL was detected when the LOD score exceeds the significance threshold, in a Linkage group. The estimated position of the QTL on the map correlates with the highest LOD value on the LG or chromosome. The significance threshold of the LOD score was determined to be 3.0, with the permutation test with a P-value of 0.05.

Then the multiple QTL mapping (MQM mapping, or composite interval mapping) module was performed: use of markers close to QTLs as cofactors to obtain the best possible final position of the QTLs.

One major QTL was identified on linkage group 6 with the favorable allele originated from the LT17 source (FIG. 3). No QTL was identified on chromosome 4, known to harbor a major QTL for Leveillula taurica resistance in other sources (PBC167).

The results in Example 2 have shown that the known QTL1/8 for resistance to Leveillula taurica does not seem to be implied in the resistance to powdery mildew in the LT17 plants. The QTL mapping experiments also show that the known major QTL4 is not present in LT17. Accordingly, the newly identified QTL on linkage group 6 seems to provide resistance to Leveillula taurica, even in the absence of two known major QTLs for resistance to Leveillula taurica on linkage group 1/8 and linkage group 4.

TABLE 7
Data for the QTL identified on chromosome 6

| | QTL | | | |
|---|---|---|---|---|
| | position | | Confidence | % of |
| Linkage | (LOD max) | | Interval in | explained |
| group | (cM) | LOD Max | cM (LOD-1.5) | variance |
| 6 | 157.4 | 5.04 | 147-160 | 9.6 |

Confidence interval was estimated based on the maximum LOD value minus 1.5.

### Example 6: Validation of QTL Effect in F2:3 Families

Based on molecular profile at QTL position on chromosome 6, F2 plants of the cross (Maor×LT17) were selected and selfed to produce F2:3 families. A total of 25 F2 plants were selected, 12 being homozygous for favorable allele inherited from LT17 and 13 being homozygous for unfavorable allele inherited from Maor at the region of the QTL located on chromosome 6.

Three different SNP markers were used to identify molecular profile at chromosome 6 QTL position. The three SNPs were selected within the interval of the QTL on chromosome 6. One SNP was selected at the left side of the interval, one SNP corresponding to the SNP with the highest significance and one SNP at the right side of the QTL interval.

SNP information are provided in Table 8 below.

TABLE 8
SNP markers for the QTL identified on chromosome 6

| | | | |
|---|---|---|---|
| SNP Name | PE-0014628 | PE-0056542 | PE-0021476 |
| Linkage | 6 | 6 | 6 |
| Group | | | |
| Position (cM) | 150.97 | 157.42 | 158.15 |
| LOD | 3.89 | 5.04 | 4.16 |
| Physical | 6 | 6 | 6 |
| chromosome | | | |
| on CM334 | | | |
| v.1.55 | | | |
| Physical | 232 015 212 | 233 673 276 | 235 235 179 |
| position on | | | |
| CM334 v.1.55 | | | |
| Sequence | AAGATACTCTG | ATTTTCTAGGTT | CTCCAAAACCA |
| | CATTGGGTCCG | ACTGGATTTAAC | CAGCTACTAATT |
| | GGCTAGAAGAA | TCAGGAGAAAA | TGTTAGATCCAA |
| | TAAGCACGAAT | TGGATTTGTTTT | AACTTAAAAGCT |
| | AACCGC[A/G]TT | TGCAATGCATTT | TAT[C/G]TTTAGT |
| | CCAAGAAAATA | TTCCGGGGGTG | CAGTCAATTAAA |
| | GATTTGGTTGA | CCTAGTTTATAA | AATCCCAACAAT |
| | GGAATTTGATC | AGACCTAAGAT | TTAAAAGCTAAG |
| | GAACACTTGGT | GTTTCAGTCAA | AAGTGAAG |
| | GGGC (SEQ ID | CTGCTTAATTAG | (SEQ ID NO: 37) |
| | NO: 1) | GACCTTTGACT | |
| | | GTTGTGGTTAT | |
| | | GACTGGGCCCC | |
| | | T[T/C]GCCAAGA | |
| | | TGGTTTTGGAC | |
| | | AACAGAGCTCT | |
| | | TACCACACTTG | |
| | | CTGCTATTTTGA | |
| | | TCTACTTGATCT | |
| | | TGAGTTGCGCT | |
| | | GTTTGCTGTGC | |
| | | TGTTCAAAGCG | |
| | | ACATTGACTGT | |
| | | CTGAGATCTATA | |
| | | AAAGATTCATTA | |
| | | GAAGATCCAAA | |
| | | TAATACT (SEQ | |
| | | ID NO: 5) | |
| Forward | GAAGGTGACCA | GAAGGTGACCA | GAAGGTGACCA |
| primer- | AGTTCATGCTA | AGTTCATGCTG | AGTTCATGCTT |
| resistant | GAAGAATAAGC | TGGTTATGACT | GTTGGGATTTTT |
| allele | ACGAATAACCG | GGGCCCCTC | AATTGACTGACT |
| | CA (SEQ ID NO: | (SEQ ID NO: 7) | AAAG (SEQ ID |
| | 3) | | NO: 39) |
| Forward | GAAGGTCGGAG | GAAGGTCGGAG | GAAGGTCGGAG |
| primer- | TCAACGGATTA | TCAACGGATTG | TCAACGGATTG |
| susceptible | GAAGAATAAGC | TGGTTATGACT | TTGGGATTTTTA |
| allele | ACGAATAACCG | GGGCCCCTT | ATTGACTGACTA |
| | CG (SEQ ID NO: | (SEQ ID NO: 6) | AAC (SEQ ID NO: |
| | 2) | | 38) |
| Common | AATTCCTCAACC | TAAGAGCTCTG | CTCCAAAACCA |
| Reverse | AAATCTATTTTC | TTGTCCAAAAC | CAGCTACTAATT |
| unlabelled | TTGGAA (SEQ ID | CATCTT (SEQ ID | TGTT (SEQ ID |
| | NO: 4) | NO: 8) | NO: 40) |
| Allele LT17 | A/A | C/C | C/C |
| (resistant) | | | |
| Allele Maor | G/G | T/T | G/G |
| (susceptible) | | | |

The 25 F2:3 families were evaluated in tunnel under artificial inoculation in 2 different geographical locations, using two different local strains, SRY and Fondi. In both location each F2:3 family was evaluated in 2 replicated plots of 5 plants each.

In the first location, evaluation of disease was performed using a simplified scoring scale.

TABLE 9
simplified scoring scale
Simplified scoring scale: 1 note by plants: bottom/medium leaves

| | Percentage | |
|---|---|---|
| | of plants with | |
| Note | mycelium | Descriptions |
| 9 | 0 | nothing |
| 8 | 0* | Very few spots of penetration (and not |
| | | mycelium) |
| 7 | 1-10% | % of the infected leaves area (Mean of the |
| 6 | 10-20% | more infected leaves) |
| 5 | 20-30% | |
| 4 | 30-40% | |
| 3 | 40-50% | |
| 2 | 50-60% | |
| 1 | >60% | |

Each plant was scored at 29 dpi, 50 dpi and 71 dpi and AUDPC was calculated as described in Example 3.

In the second location, evaluation of disease was performed only once according to the same simplified scoring scale.

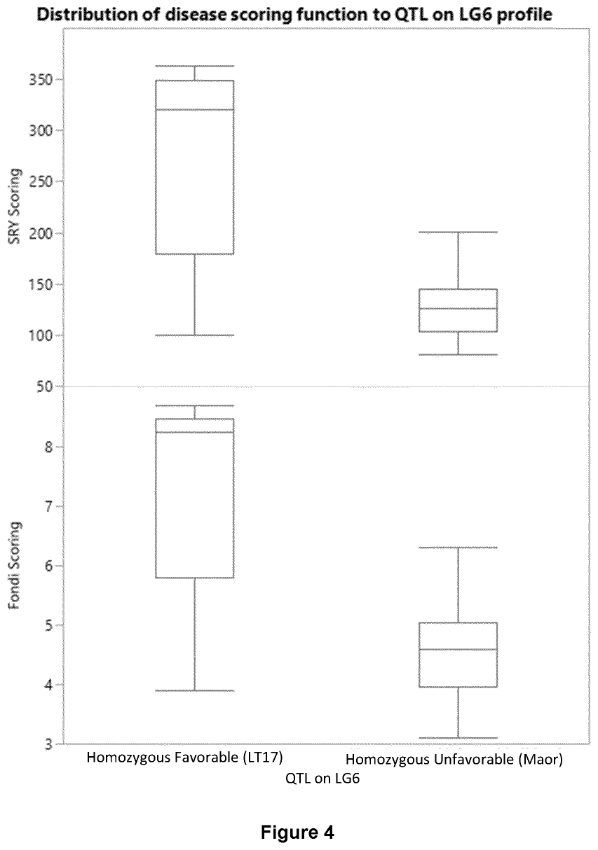

FIG. 4 represents the distribution of disease scoring at the 2 locations for the F2:3 families according to their genotype profile at the QTL location on chromosome 6 (homozygous for favorable allele versus homozygous for the unfavorable allele).

At both location, significant differences were observed between means of disease scoring according to QTL profile. This validates the QTL effect within the genetic background in which it was initially identified. These experiments also show that the identified QTL provides resistance to different strains of Leveillula taurica.

### Example 7: Candidate Genes Identification within QTL Interval

The confidence interval of the QTL corresponds to 3.3 Mb on the reference genome CM334 v1.55 between 232.0 Mb and 235.3 Mb on the chromosome 6. Based on physical genome gene annotation from gene prediction tools, a total of 410 genes were found within the interval.

Among all the genes found in the QTL interval, two genes have been found as good candidate genes based on their molecular functions and their physical position proximity with the peak value of the QTL (maximum LOD value).

One MLO-like protein gene (MLO-like protein, gene name=CA06g26150 in C. annuum cv. CM334 v.1.55 genome) and one receptor-like kinase (probably inactive leucine-rich repeat receptor-like protein kinase gene, name=CA06g26250 in C. annuum cv. CM334 v.1.55 genome).

Implication of other Mlo genes or Mlo-like sequences in the resistance of fungus and in particular for Leveillula in pepper has been described (Zheng Zheng et al 2013, Lim and Lee (2014), Kim and Hwang (2012)).

### Example 8: Markers Development within Candidate Genes

The 2 candidates genes were sequenced by targeted sequencing by in solution fragment capture (Gnirke et al., Nat Biotechnol. 2009). Based on sequences data for a set of 6 genotypes carrying or not the full favorable segment at QTL interval, SNP mining was conducted.

Several genotypes were sequenced for the 2 candidates genes: Maor, CPNT, Vania, BYP, LT17, F3 (LT17*Maor) and HV12.

A total of 8 SNP were identified within or close to the 2 genes, with respectively 6 SNPs in CA06g26250 and 2 SNPs in CA06g26150.

TABLE 10
SNPs identified in CA06g26250 and CA06g26150.

| | | Position | Ref | | | | | | | | |
|---|---|---|---|---|---|---|---|---|---|---|---|
| | | CM334 | Allele | Alt | | | | | | | |
| Gene | Chromosome | v1.55 | (=Maor) | Allele | Maor | CPNT | Vania | BYP | LT17 | F3 | HV12 |
| CA06g26250 | 6 | 233 673 | T | C | . | . | . | . | C/C | C/C | C/C |
| | | 276 | | | | | | | | | |
| CA06g26250 | 6 | 233 673 | G | A | . | . | . | . | A/A | A/A | . |
| | | 121 | | | | | | | | | |
| CA06g26250 | 6 | 233 672 | G | T | . | . | . | . | T/T | T/T | . |
| | | 530 | | | | | | | | | |
| CA06g26250 | 6 | 233 671 | T | C | . | . | . | . | C/C | C/C | . |
| | | 922 | | | | | | | | | |
| CA06g26250 | 6 | 233 671 | A | C | . | . | . | . | C/C | C/C | . |
| | | 648 | | | | | | | | | |
| CA06g26250 | 6 | 233 671 | A | C | . | . | . | . | C/C | C/C | C/C |
| | | 204 | | | | | | | | | |
| CA06g26150 | 6 | 233 493 | T | A | . | . | . | . | A/A | A/A | . |
| | | 124 | | | | | | | | | |
| CA06g26150 | 6 | 233 489 | C | T | . | . | . | . | T/T | T/T | . |
| | | 782 | | | | | | | | | |

“.” means that the plant homozygously comprises the Ref Allele.

For each of these SNP, Kaspar markers were developed, as shown in Table 11 below.

TABLE 11
SNP markers in or near genes CA06g26250 and CA06g26250

| | | Physical | | | Forward | | | |
|---|---|---|---|---|---|---|---|---|
| SNP | | position | | Forward primer | primer | Common | | |
| marker | | CM334 | | (susceptible | (resistant | reverse | Susceptible | Resistant |
| Name | Gene | v.1.55 | Sequence surrounding the marker | allele) | allele) | primer | allele | allele |
| PE- | CA06g26502 | 233 673 | ATTTTCTAGGTTACTGGATTTAACTCAGGAGAAAATGGAT | GAAGGTCGGA | GAAGGTGA | TAAGAGCT | T/T | C/C |
| 0056542 | | 276 | TTGTTTTTGCAATGCATTTTTCCGGGGGTGCCTAGTTTAT | GTCAACGGATT | CCAAGTTC | CTGTTGTC | | |
| | | | AAAGACCTAAGATGTTTCAGTCAACTGCTTAATTAGGACC | GTGGTTATGAC | ATGCTGTG | CAAAACCA | | |
| | | | TTTGACTGTTGTGGTTATGACTGGGCCCCT[T/C]GCCAAG | TGGGCCCCTT | GTTATGACT | TCTT (SEQ | | |
| | | | ATGGTTTTGGACAACAGAGCTCTTACCACACTTGCTGCTA | (SEQ ID NO: 6) | GGGCCCCT | ID NO: 8) | | |
| | | | TTTTGATCTACTTGATCTTGAGTTGCGCTGTTTGCTGTGC | | C (SEQ ID | | | |
| | | | TGTTCAAAGCGACATTGACTGTCTGAGATCTATAAAAGAT | | NO: 7) | | | |
| | | | TCATTAGAAGATCCAAATAATACT (SEQ ID NO: 5) | | | | | |
| PE- | CA06g26250 | 233 673 | AGATGGTTTTGGACAACAGAGCTCTTACCACACTTGCTG | GAAGGTCGGA | GAAGGTGA | ATGAAACC | G/G | A/A |
| 0056543 | | 121 | CTATTTTGATCTACTTGATCTTGAGTTGCGCTGTTTGCTG | GTCAACGGATT | CCAAGTTC | TTCCGTCA | | |
| | | | TGCTGTTCAAAGCGACATTGACTGTCTGAGATCTATAAAA | CATTAGAAGAT | ATGCTAGA | GATTATCG | | |
| | | | GATTCATTAGAAGATCCAAATAATACTTTGA[G/A]CTATAC | CCAAATAATAC | TTCATTAGA | AACTT (SEQ | | |
| | | | ATGGAAGTTCGATAATCTGACGGAAGGTTTCATTTGCAAG | TTTGAG (SEQ | AGATCCAA | ID NO: 12) | | |
| | | | TTTGCTGGGATTCAGTGCTGGCATCCTGATGAAAATAAG | ID NO: 10) | ATAATACTT | | | |
| | | | GTTCTGAGTATCAGTCTTCCTGACATGGGTCTCAAGGGC | | TGAA (SEQ | | | |
| | | | AGGTTTCCACTTGGTCTTCGGAATTG (SEQ ID NO: 9) | | ID NO: 11) | | | |
| PE- | CA06g26250 | 233 672 | CTGTTCCTGCAGAGAGCTATGCCAATAATCCCGGACTGT | GAAGGTGACCA | GAAGGTCG | TTCCTCTTC | G/G | T/T |
| 0056544 | | 530 | GTGGAGAACCGTTAGATCCTTGCCAGGGTACTTCTAAGA | AGTTCATGCTG | GAGTCAAC | CTGGATAT | | |
| | | | AACCCCCTACCGCAATCATTGTCGGTGCAGCTGTTGGTG | GTGCTGTATTT | GGATTAGG | CCTTCTCAT | | |
| | | | GGGTAAGTTTAGGTGCTGTATTTTTGGCCATCG[G/T]TAT | TTGGCCATCGG | TGCTGTATT | ATA (SEQ | | |
| | | | GTTCTTCTATATGAGAAGGATATCCAGGAAGAGGAAGAA | (SEQ ID NO: 14) | TTTGGCCAT | ID NO: 16) | | |
| | | | GGATGATGATCCTGAAGGGAATAAATGGGCGAAGAGTAT | | CGT (SEQ | | | |
| | | | TAAGGGTAAAAAGGCAATCCAGGTAAGAATTTTAACTTCT | | ID NO: 15) | | | |
| | | | CTGAATCATTAAAAACTGTCTCGTTACTG (SEQ ID NO: 13) | | | | | |
| PE- | CA06g26250 | 233 671 | TCTTTTCATAGAAGCATTTAACTAATGAATTTTCACCTGCT | GAAGGTCGGA | GAAGGTGA | GTTTGAGA | T/T | C/C |
| 0056545 | | 922 | CCGCTGCTCGTGTGTGATTTTTCTCTTGTTCAAGTTTTCT | GTCAACGGATT | CCAAGTTC | AGTCTACTT | | |
| | | | TAATTGACTTAAGGTTCCTTATTTTCAGCTTTCAATGTTTG | CTGTCAGTGGC | ATGCTGTC | CAAAAATG | | |
| | | | AGAAGTCTACTTCAAAAATGAGATTTAG[T/C]GATCTTATG | CTTCATAAGAT | AGTGGCCT | AGATT (SEQ | | |
| | | | AAGGCCACTGACAGCTTCAACAAAAACAATATTATCGGGT | CA (SEQ ID NO: | TCATAAGAT | ID NO: 20) | | |
| | | | CAGGAAGAACCGGGACTTTTTACAAAGCAGTACTTGATG | 18) | CG (SEQ ID | | | |
| | | | ATGGCACTTCACTTATGGTTAAGAGGTTGCAGAATACTCA | | NO: 19) | | | |
| | | | GCACTCAGAGAAAGAGTTTATG (SEQ ID NO: 17) | | | | | |
| PE- | CA06g26250 | 233 671 | ACTCAGCACTCAGAGAAAGAGTTTATGTCCGAGATGGCT | GAAGGTGACCA | GAAGGTCG | TTGGTCTAC | A/A | C/C |
| 0056546 | | 648 | ACATTGGGAAATGTAAAGCATCGTAATCTGGTTCCTCTTT | AGTTCATGCTC | GAGTCAAC | AAAGACAT | | |
| | | | TAGGTTTCTGCATTGCCAAAAAAGAAAGGCTGTTGGTCTA | CTTCACTCACC | GGATTCCTT | GCCAAATG | | |
| | | | CAAAGACATGCCAAATGGAACCTTGCATGAT[A/C]GGTTA | GAATGTAACCT | CACTCACC | GAA (SEQ | | |
| | | | CATTCGGTGAGTGAAGGCGAAAAAACTCTAGAGTGGCCT | (SEQ ID NO: 22) | GAATGTAA | ID NO: 24) | | |
| | | | ATAAGATTAAAAATAGGCATTGGAGCAGCCAAAGGATTTG | | CCG (SEQ | | | |
| | | | CATGGCTTCACCACAACTGCAATCCTCGTATCATTCACAG | | ID NO: 23) | | | |
| | | | AAATATTAGTTCTAAATGCATCTTGC (SEQ ID NO: 21) | | | | | |
| PE- | CA06g26250 | 233 671 | GCTACTCCAAAAGGTGATGTGTACAGTTTCGGTGTTGTA | GAAGGTGACCA | GAAGGTCG | CATTGTCAT | A/A | C/C |
| 0056547 | | 204 | CTTCTCGAGTTAGTTACTGGTGAGAAACCGACCTCTGTC | AGTTCATGCTC | GAGTCAAC | AACCTTTAC | | |
| | | | ACCAAAGCTCCCGAGACCTTTAAGGGGAATTTGGTGGAA | AACTCTCCGGT | GGATTAAC | CAGACAAT | | |
| | | | TGGATCACACAACTCTCCGGTGAATCTAAGCTT[A/C]AAG | GAATCTAAGCT | TCTCCGGT | GAAT (SEQ | | |
| | | | ATGCGATTGACCATTCATTGTCTGGTAAAGGTTATGACAA | TA (SEQ ID NO: | GAATCTAA | ID NO: 28) | | |
| | | | TGAGATCTTCCAGGTCCTTAAAGTTGCTTGTCGATGTGTG | 26) | GCTTC | | | |
| | | | TTGTCTCCTGTTCCTAAGGAGAGGCCGACAATGTTTGAA | | (SEQ ID NO: | | | |
| | | | GTTTACCAGCTTCTGAGAGCCATCGGAG (SEQ ID NO: 25) | | 27) | | | |
| PE- | CA06g26150 | 233 493 | TAACTGATGGTTTCAGTTGAGCTGCAAAATGGAACATCAA | GAAGGTCGGA | GAAGGTGA | GAGTTTCAT | T/T | A/A |
| 0056549 | | 124 | TATCAGTAGCTTCTCGTTTCAACCATTTACTTCCTTTGCTC | GTCAACGGATT | CCAAGTTC | GATTCATCT | | |
| | | | TTTAGTCACTTGCACTTTAGAGAAGAATGACGTGATTCCC | CCGATATTTTAT | ATGCTCCG | GTTTTGTTT | | |
| | | | GATATTTTATACTTAGCTATTCTAGCATA[T/A]GGGAAACA | ACTTAGCTATT | ATATTTTAT | CCC (SEQ | | |
| | | | AAACAGATGAATCATGAAACTCTTCGGTAGTAAAAAAAAT | CTAGCATAT | ACTTAGCTA | ID NO: 32) | | |
| | | | TATGTGCCAAAATGTTGGATGTATCTTTTTATGGACGATG | (SEQ ID NO: 30) | TTCTAGCAT | | | |
| | | | ATCCATACCATGTTCGGATTCAGGATCCACTTTTACGATT | | AA (SEQ ID | | | |
| | | | GCATCCTCCCAGCTCTTCCATT (SEQ ID NO: 29) | | NO: 31) | | | |
| PE- | CA06g26150 | 233 489 | TTTGATTTTGTTTAACTCATTTGCATGAAATGATATGTTTA | GAAGGTGACCA | GAAGGTCG | TAGGTAATA | C/C | T/T |
| 0056550 | | 782 | TATGTTTGTCTAGAAATATCACAACTATTACATTTATAGGT | AGTTCATGCTA | GAGTCAAC | TTTGAGTTG | | |
| | | | AATATTTGAGTTGCACTTATGGTATAGTGCATTTTTATTTC | CATTAATTTTCC | GGATTGAA | CACTTATG | | |
| | | | TATTTATGTGACATTATTTTTTAGATA[C/T]ATGTCGAAATA | AGCATTATTTC | CATTAATTT | GTAT (SEQ | | |
| | | | ATGCTGGAAAATTAATGTTCAACTCTTTGAATACATGAAA | GACATG (SEQ | TCCAGCATT | ID NO: 36) | | |
| | | | GAACCGAAACTTCTACCCTATTTTTAAGGGATGGACGCG | ID NO :34) | ATTTCGACA | | | |
| | | | TGAATTAGACCTAATTAAGAAATATTTATCTTACACTTTTT | | TA (SEQ ID | | | |
| | | | GTTTTATCGAGAAGCTTTT (SEQ ID NO :33) | | NO: 35) | | | |

### Example 9: SNP Analysis on Chromosome 6

Example 8 shows that the LT17 line has distinct alleles at the identified QTL when compared with the reference Maor line, but also the CPNT, Vania and BYP lines. The LT17 line is also distinct from the resistant HV12 line at the identified QTL on chromosome 6. In order to further characterize the LT17 lines, the inventors performed a SNP metachip analysis on chromosome 6 of the CM334 v.1.55 reference genome. The inventors identified 31 SNPs for which the LT17 line has a different allelic state in comparison to the Leveillula taurica resistant PBC167 line. This confirms that the QTL identified on chromosome 6 is unique compared to the known resistant lines.

TABLE 12
SNP analysis on chromosome 6

| | | | | Position in | | |
|---|---|---|---|---|---|---|
| SNP | | Sequence | | bp (CM334 | | |
| marker | Sequence | Orientation | Chromosome | v. 1.55) | LT17 | PBC167 |
| PE- | TTGAAACCCTCCTTCCTTTCATACATTGAAAGAAATGGTGGATGGAGACT[C/G]AG | Top | 6 | 219 426 890 | C/C | G/G |
| 0011447 | CAAATCATTGGTCACATCATACAACCTCAAGAGCATACTAGAAGCTGA (SEQ ID | | | | | |
| | NO: 46) | | | | | |
| PE- | AGTTGCCTTGGTACCAATGAAATTGACTCTTCTTTTCCTTCCAAGTCCAC[A/G]GC | Top | 6 | 219 425 984 | A/A | G/G |
| 0011448 | TCCAGGACCTGCATTCAGATCCAGACCTTGCCGGCTTCGGCTTCTGTT (SEQ ID | | | | | |
| | NO: 47) | | | | | |
| PE- | GGTGGTAGAATCCTCCCTCGTCGCTCTGACGGCACGCTCCGTTACATCGG[A/C]G | Top | 6 | 220 448 196 | C/C | A/A |
| 0019751 | GTTTTACTAGAGTCCTCTCCGTTGATCGGTCTATTTCTTATTCAGAGTT (SEQ ID | | | | | |
| | NO: 48) | | | | | |
| PE- | TGGTTCTAATGTGGTGACAAAGGTTCCATCAAGTGAATGTTATGGTGATG[G/T]TG | Bottom | 6 | 219 581 697 | T/T | G/G |
| 0021027 | GTGTAAGTGCTAACGTGCAGAAGAGTAAAGCAAATGAAGCAAACGGGG (SEQ ID | | | | | |
| | NO: 49) | | | | | |
| PE- | GAGATAGAAAATGAAGGTGAACAAAACTTTGGATCTCCAGCTGCACCAGT[C/T]CA | Bottom | 6 | 219 545 686 | T/T | C/C |
| 0022402 | GGAACGACCAATTCAGAGATCATGGGTTCCTCCCCAGCCTCCTCCAGT (SEQ ID | | | | | |
| | NO: 50) | | | | | |
| PE- | AAAAGAATCCGCCCTAACTGCTTTCTGCTCTTCTTCTTCACATTGACTTC[A/C]TCG | Top | 6 | 224 014 874 | C/C | A/A |
| 0018388 | GCATCATCTAGAACCAAATTCATGTACTCATCAAAACCAATAATACG (SEQ ID NO: | | | | | |
| | 51) | | | | | |
| PE- | ATAATGATGTCACCAGTCTGAACTACCGAAGACAGTTCTTTGCTTCAATC[A/G]AT | Top | 6 | 225 012 078 | G/G | A/A |
| 0028409 | ATGCTACAAGAACAAGCTGAGAAATTACTTGAAGAGATGAAGCCTAAG (SEQ ID | | | | | |
| | NO: 52) | | | | | |
| PE- | GTCCCTCTTTCAAGAGTTAAAGAAGTGGTGTGTGGGATTTGCTTTGACTACTACC | Top | 6 | 225 782 942 | G/G | A/A |
| 0006573 | CGCCT[G/A]ATGACATAATGTCTGTTGGTTGTGGTCATCCTTTTTGTACTATTTGCT | | | | | |
| | GGAAAGCTTACA (SEQ ID NO: 53) | | | | | |
| PE- | AKTAAAGAGAACACCTGTATTCACCAGTACTACTACCCAGAATGGGTCAATTTAC | Bottom | 6 | 227 119 514 | T/T | G/G |
| 0001206 | ACC[T/G]ATCTTGGACAAAATGATGACATCCATCCCTCCTATCTTATTGAAGCATG | | | | | |
| | CTACATTCAGTCTTGCACTTTGACAGATAAGTGTTTCACCACATATCCAATAAGAT | | | | | |
| | GCACACCATATAACATTGATGCTGGCAAGAGTATGCACCTGCAG (SEQ ID NO: | | | | | |
| | 54) | | | | | |
| PE- | GGAGGCAACAACACAAAGTATCCCACTACCCCCCACTAAGATGGGGATAGTGAA | Top | 6 | 227 195 529 | C/C | A/A |
| 0005987 | ACAAGG[A/C]ATAGTGTCCATTCTTGGATCAGACTCTGACAAAAGAAACAAGGCTG | | | | | |
| | CTGCAGCTGTTTCA (SEQ ID NO: 55) | | | | | |
| PE- | GAACGCTTATGGGATTGTTCCAGGGGTAAAACATTACGCTTGCTTAGTTGATATA | Bottom | 6 | 232 016 457 | T/T | C/C |
| 0005367 | CTTGG[C/T]CGTGCAGGTGAACTGGAAGAAGCTGAAGAACTGATAAATAATATGC | | | | | |
| | CAGTAACCCCTGGT (SEQ ID NO: 56) | | | | | |
| PE- | AAGATACTCTGCATTGGGTCCGGGCTAGAAGAATAAGCACGAATAACCGC[A/G]T | Top | 6 | 232 015 212 | A/A | G/G |
| 0014628 | TCCAAGAAAATAGATTTGGTTGAGGAATTTGATCGAACACTTGGTGGGC (SEQ ID | | | | | |
| | NO: 1) | | | | | |
| PE- | CGGATATTGGAGAAGATGAAGGCTCCTAAAAAGCCAGATGATGTGCCTGAGATC | Top | 6 | 232 005 084 | A/A | G/G |
| 0006634 | ACGCCT[G/A]AGCAGCTCTTGGAAGCTGATGGTTTCATCTTTGGTTTCCCTTCGCG | | | | | |
| | TTTTGGTGTGATGG (SEQ ID NO: 57) | | | | | |
| PE- | TCGGTTGCTTGCAGGTTATTGCAGTATTGGTGAGACCAGACAAGGAATCAAAAAT | Top | 6 | 233 577 814 | A/A | G/G |
| 0006718 | AAGAA[G/A]ATACTGGAATTGGTACCATTACACAGCAGGAAGAATTTTGATATTATT | | | | | |
| | GGCATCAATAAA (SEQ ID NO: 58) | | | | | |
| PE- | ATGTCTTACAAACCTCCATTGACAACAAAGATTACATTGCGGGCAAACTC[C/G]TC | Bottom | 6 | 235 354 680 | G/G | C/C |
| 0012892 | CACGCCCATATACTCCGACTTGGCCTTTCCTCTCATACACTCCTCCTA (SEQ ID | | | | | |
| | NO: 59) | | | | | |
| PE- | TATACAAGASAATGTTGGAAGGGGAATGGGAAAATGGAATATCAGCTGGCACAAT | Bottom | 6 | 235 302 145 | A/A | T/T |
| 0004191 | TGTCC[T/AJAGCAGCAGTAGCTTTATTGCTTGATGGAACATCATTGTATAGATTCAT | | | | | |
| | GAAGGCAGAGGG (SEQ ID NO: 60) | | | | | |
| PE- | TGTGTAGTTGTTGGCATTAAAATTTGCTTCTAGCCTTTCAGAAAAGACATTACGTT | Top | 6 | 235 289 831 | A/A | C/C |
| 0005575 | GTAA[A/C]CTTGTAGTGTGTATTTAATTCTCTTGACATGAAATGAAGTCGAGTGGA | | | | | |
| | CACAACAAAAAA (SEQ ID NO: 61) | | | | | |
| PE- | CTAGGCCTAGTTGATGGGACTACACAATGGTATGCATGCAAACTAAGCAAAAGAT | Top | 6 | 235 287 024 | A/A | G/G |
| 0006320 | GGGGC[A/G]CGATGGAGGAAAAAGATCTGCATACCAGCAACTTGTTGGGCATATA | | | | | |
| | TTTTAGTCATGCTA (SEQ ID NO: 62) | | | | | |
| PE- | GAATGTTTTTGCGTTTTGTGGTACCACACTCTCTTGATCATGCTCAAATG[C/T]TTC | Bottom | 6 | 235 290 056 | T/T | C/C |
| 0016499 | AAAAGCATACATGCATTTAAAGAATCCTTCAGCAGCAAATCCAGAAG (SEQ ID | | | | | |
| | NO: 63) | | | | | |
| PE- | TGAATCCAAACAGATTTGGGACACTGACTGGGACTGATGAACTCATAAGT[A/C]TT | Top | 6 | 235 238 619 | A/A | C/C |
| 0013625 | GCCTTTCTTTGCCTCCTTTTGTTTCGTTTTCTTCCTTTCTGTTGGTTA (SEQ ID NO: | | | | | |
| | 64) | | | | | |
| PE- | TGATCATAATATCAAATCTGATGTTCAACAATACAATATGCACAAGAGGA[A/G]AAA | Top | 6 | 235 238 219 | A/A | G/G |
| 0022671 | AACTGGTTCATAAATGGTGAATCAAGAAGGCAATACGACGAGCCTTT (SEQ ID | | | | | |
| | NO: 65) | | | | | |
| PE- | CTCCCGTCATCTGTTAAATGCTTCATTGCTTCAGACATTATTAAGTGCTAGAACTT | Bottom | 6 | 234 323 803 | C/C | T/T |
| 0005787 | CAGG[C/T]GTTCGTTCACCTTGTTATATTGTACATGACAATGTTGATGCATGTGGT | | | | | |
| | AGCATTTGTTTG (SEQ ID NO: 66) | | | | | |
| PE- | TACTCCTTCAGGTGGATCTAAGGGTGAACCAAGTGGAAATTCAGGATACA[C/G]A | Top | 6 | 234 848 256 | C/C | G/G |
| 0020909 | GTCAAGCTGCCATATTCTCTCTTGTTTACCCTTGTAGTTGCAGCATCAT (SEQ ID | | | | | |
| | NO: 67) | | | | | |
| PE- | TTGAATTAGCTGGTAATGCAAGCAAGGATCTTAAAGTGAAGAGAATCACCCCCAG | Top | 6 | 235 746 196 | A/A | G/G |
| 0000401 | GCACTTGCAACTGGCCATTCGCGGAGACGAGGA[A/G]CTTGACACACTTATCAAA | | | | | |
| | GGGACCATTGCTGGTGGTGGTGTCATCCCTCACATTCACAAGTCCCTAATAAACA | | | | | |
| | AAACCTCCAAGGAGTGAATTTCTCCTTTTTtATCTTAGATGTATCATG (SEQ ID NO: | | | | | |
| | 68) | | | | | |
| PE- | TTAAATGTTCAACAAGAGTCCATTCTACAGCACAATGAGCAAGTTTGGTAAAGGG | Bottom | 6 | 235 888 699 | T/T | C/C |
| 0003984 | CCAA[C/T]GCGATGAGCCTGTTTGCTTTATATACTTACGGAGGAAAACTCAAACAT | | | | | |
| | GTGATAAGTACA (SEQ ID NO: 69) | | | | | |
| PE- | GTTGCTTAGTCTAAGAGAGACAATAGCTCTTTTTAAGCTTAATTCACCAC[C/T]ATT | Bottom | 6 | 236 289 525 | C/C | T/T |
| 0014672 | ATCTACTTGCACTTGAAAAGTCCAAAGAGAAAAAAGGGACCAAAACT (SEQ ID | | | | | |
| | NO: 70) | | | | | |
| PE- | ACAATAGTTTTATCTCCAGCACTAAAATGGTTCAGCACCTGTGTTCTGTGAAGTAA | Top | 6 | 236 546 851 | G/G | A/A |
| 0005702 | TGTG[A/G]ACAATTTTGTGTAGGATTTCCATGTACCTGCAATTGAATCACATATTAC | | | | | |
| | AGGCGGAGTTA (SEQ ID NO: 71) | | | | | |
| PE- | CTAATAATCAAGATGAGAATATTAATATGGTGCAATCAGCGGATGATGAT[A/T]TAC | Top | 6 | 236 574 405 | A/A | T/T |
| 0021582 | AAATGTTGCAGCAGCAGCCTACTGATTTTGACCCAATGGAGGATACT (SEQ ID | | | | | |
| | NO: 72) | | | | | |
| PE- | GTGAAGTTATGGAAATAAATGAGTAGAGGATCTGATGTCGGAGCAAGTAGGGTA | Top | 6 | 236 762 040 | A/A | C/C |
| 0007248 | AAGAGT[C/A]TCGCGTCTCATGTTGTAGTGTATTGTCATCATAAATTTCGATTTTTC | | | | | |
| | TGCTTCTTAGTCA (SEQ ID NO: 73) | | | | | |
| PE- | CTCAAATAGCCGGGAATTCATTACATTTTCCCCTTGTTATAACATTATCAATACTG | Bottom | 6 | 236 880 605 | T/T | C/C |
| 0003669 | AGCT[C/T]CTATTCTTGACTGACTATGCACTCAGGTAGGAATTCATACATACAAACT | | | | | |
| | CCCCAAAAAGG(SEQ ID NO: 74) | | | | | |
| PE- | GTTGCTTAGTCTAAGAGAGACAATAGCTCTTTTTAAGCTTAATTCACCAC[C/T]ATT | Bottom | 6 | 236 289 525 | C/C | T/T |
| 0014672 | ATCTACTTGCACTTGAAAAGTCCAAAGAGAAAAAAGGGACCAAAACT (SEQ ID | | | | | |
| | NO: 75) | | | | | |

### Example 10: Validation of the Effects of QTL on Chromosome 6 in Susceptible Background Though QTL Introgression and Evaluation of Dominance Effects

A three-way hybrid F2 population was created as follow: The LT17 source was crossed by an intermediate genotype (HPG117) and then the obtained hybrid was crossed again by a susceptible bell pepper line (BYP). This 3-way hybrid was then selfed to obtain a F2 population segregating for the QTL on chromosome 6 within susceptible background.

Based on the markers described in examples 6 and 8, a total of 180 F2 plants were genotyped to select different plants with different genotype profile in the QTL region. A total of 20 F2 plants were selected for each of the 3 following QTL profile: homozygous favorable allele, homozygous unfavorable allele and heterozygous.

All selected F2 plants were evaluated in artificial test following the protocol described in Example 2. Selected plants having the same QTL profile were grouped in two plots of 10 plants organized in 2 repetitions of 5 plants each, leading to a total of 6 F2 plots of 10 plants. In addition to the selected F2s, 1 F4 (Maor*LT17) genotype being homozygous for favorable alleles was included in the test, as described below in Example 11.

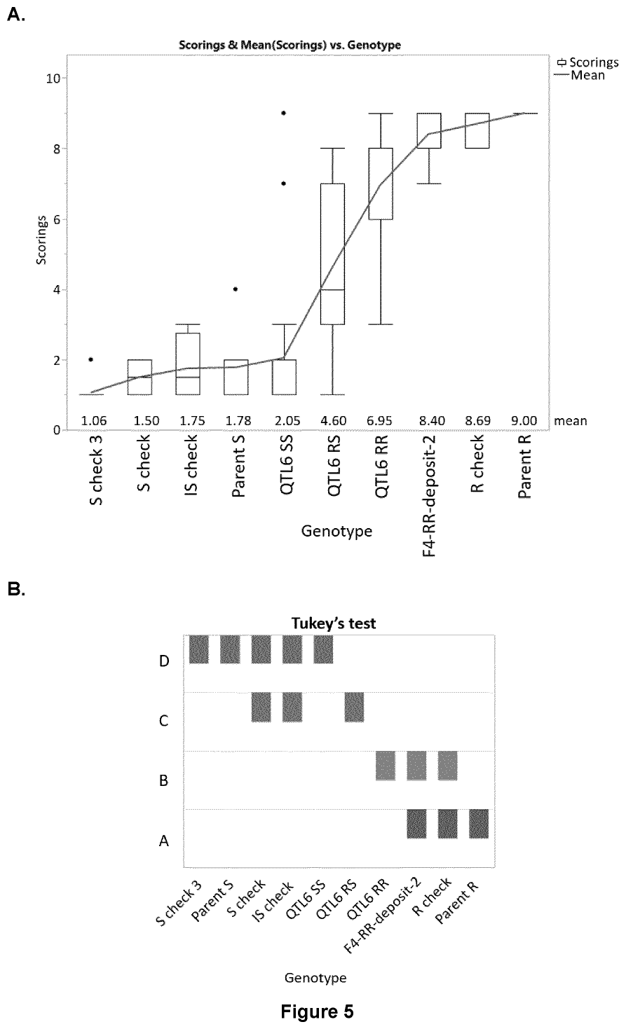

Results are presented in FIG. 5. Results show a clear effect of QTL6 with an additive effect. For the F4 (Maor*LT17) the seed lot F4 (ST117F04-1157/BLK) is well resistant. Seeds of the seed lot F4 (ST117F04-1157/BLK) were thus selected for deposit at the NCIMB under accession number NCIMB 43602.

### Example 11: Validation of Candidate Genes within QTL6 Interval

F2 seeds were produced from a cross between Maor (used as female) and LT17 (used as male). F2 plants have been evaluated under artificial inoculation (as described in Example 6). F3 seeds from genotypically selected F2 plants harboring QTL6 were harvested then selfed to obtain F4 (Maor×LT17) seeds (ST117F04-1157/BLK) that were used as females pollinated with Pipo (Susceptible genotype) pollen. F1 [F4 (Maor×LT17)*Pipo] seeds were used to generate the population F2[F4 (Maor×LT17)*Pipo]. A total of 5940 F2 plants derived from this population were genotyped with markers developed within the 2 candidate genes as described in examples 6 and 8. Out of 5940 F2 plants genotyped, a total of 131 F2 plants were selected according to their genotypic profile for the 2 candidate genes in order to evaluate gene effects in artificial test. The artificial test was conducted according to the protocol described in Example 2. Plants selection were organized in plots of 10 plants to conduct the artificial test.

Below are molecular profiles of the plants, and the results obtained from artificial test scoring:

| | | | Number | |
|---|---|---|---|---|
| | Gene | Gene | of tested | Mean |
| NAME | CA06g26250 | CA06g26150 | F2 plants | score |
| F2[(Maor × | Homozygous | Homozygous | 1 | 8.0 |
| LT17)*Pipo | unfavorable | favorable | | |
| F2[(Maor × | Homozygous | Homozygous | 60 | 6.3 |
| LT17)*Pipo | favorable | favorable | | |
| F2[(Maor × | Heterozygous | Homozygous | 6 | 3.6 |
| LT17)*Pipo | | unfavorable | | |
| F2[(Maor × | Homozygous | Heterozygous | 4 | 3.5 |
| LT17)*Pipo | unfavorable | | | |
| F2[(Maor × | Homozygous | Homozygous | 60 | 2.6 |
| LT17)*Pipo | unfavorable | unfavorable | | |

These results confirm the effect of QTL and suggest a dominant mechanism involving the CA06g26150 gene encoding an MLO-like protein.

### Example 12: Genetic Modification of Capsicum Seeds by Ethyl Methane Sulfonate (EMS)

Seeds of Capsicum plants are to be treated with EMS by submergence of approximately 2000 seeds into an aerated solution of either 0.5% (w/v) or 0.7% EMS for 24 hours at room temperature.

Approximately 1500 treated seeds per EMS dose are germinated and the resulting plants are grown, preferably in a greenhouse to produce seeds.

Following maturation, M2 seeds are harvested and bulked in one pool per variety per treatment. The resulting pools of M2 seeds are used as starting material to identify the individual M2 seeds and the plants resistant to powdery mildew.

### BIBLIOGRAPHY

Daubeze, A. M. et al (1995). Resistance to Leveillula taurica in pepper (Capsicum annuum) is oligogenically controlled and stable in Mediterranean regions. Plant Breeding, 114(4), 327-332.
George, E. F. and Sherrington, P. D. (1984) Plant Propagation by Tissue Culture, Handbook and Directory of Commercial Laboratories, Exegetics Ltd
Gnirke, A. et al (2009). Solution hybrid selection with ultra-long oligonucleotides for massively parallel targeted sequencing. Nature biotechnology, 27(2), 182-189.
Hui (Y. H.), Sherkat F., Handbook of Food Science, Technology and Engineering, vol. 4, CRC Press
Kim, D. S., & Hwang, B. K. (2012). The pepper MLO gene, CaMLO2, is involved in the susceptibility cell-death response and bacterial and oomycete proliferation. The Plant Journal, 72(5), 843-855.
Lefebvre, V., Daubèze, A. M., van der Voort, J. R., Peleman, J., Bardin, M., & Palloix, A. (2003). QTLs for resistance to powdery mildew in pepper under natural and artificial infections. Theoretical and Applied Genetics, 107(4), 661-666.
Lim, C. W., & Lee, S. C. (2014). Functional roles of the pepper MLO protein gene, CaMLO2, in abscisic acid signaling and drought sensitivity. Plant molecular biology, 85(1-2), 1-10.
Molot, Jp et al. Leveillula taurica (Lév) Arn: cultures axéniques, biologie et spécificité parasitaire. Agronomie, EDP Sciences, 1990, 10 (7), pp. 551-559.
Zheng, Z. et al (2013). Loss of function in Mlo orthologs reduces susceptibility of pepper and tomato to powdery mildew disease caused by Leveillula taurica. PloS one. 8(7), e70723.

1-16. (canceled)

17. A Capsicum plant resistant to powdery mildew caused by the fungal pathogen Leveillula taurica, or a cell or plant part thereof, wherein the plant comprises introgressed in its genome a quantitative trait locus (QTL) conferring resistance to powdery mildew, wherein the QTL is located on chromosome 6 within the genomic interval delimited by the marker PE-0014628 (SEQ ID NO: 1) and the marker PE-0021476 (SEQ ID NO: 37).

18. The plant according to claim 17, wherein the QTL is present on chromosome 6 in the genome of a seed of Capsicum annuum ST17-1157, deposited at NCIMB accession number NCIMB 43602.

19. The plant according to claim 17, wherein the presence of the QTL comprises the presence of one or more of:
allele A of PE-0014628 (SEQ ID NO: 1);
allele C of PE-0056542 (SEQ ID NO: 5);
allele A of PE-0056543 (SEQ ID NO: 9);
allele T of PE-0056544 (SEQ ID NO: 13);
allele C of PE-0056545 (SEQ ID NO: 17);
allele C of PE-0056546 (SEQ ID NO: 21);
allele C of PE-0056547 (SEQ ID NO: 25);
allele A of PE-0056549 (SEQ ID NO: 29);
allele T of PE-0056550 (SEQ ID NO: 33); and/or
allele C of PE-0021476 (SEQ ID NO: 37).

20. The plant according to claim 17, wherein the presence of the QTL comprises following allele combinations:
allele combination “A” comprising allele A of PE-0056543 (SEQ ID NO: 9), allele T of PE-0056544 (SEQ ID NO: 13), allele C of PE-0056545 (SEQ ID NO: 17), and allele C of PE-0056546 (SEQ ID NO: 21);
allele combination “B” comprising allele C of PE-0056542 (SEQ ID NO: 5), allele A of PE-0056543 (SEQ ID NO: 9), allele T of PE-0056544 (SEQ ID NO: 13), allele C of PE-0056545 (SEQ ID NO: 17), allele C of PE-0056546 (SEQ ID NO: 21) and allele C of PE-0056547 (SEQ ID NO: 25);
allele combination “C” comprising allele C of PE-0056542 (SEQ ID NO: 5), allele A of PE-0056543 (SEQ ID NO: 9), allele T of PE-0056544 (SEQ ID NO: 13), allele C of PE-0056545 (SEQ ID NO: 17), allele C of PE-0056546 (SEQ ID NO: 21), allele C of PE-0056547 (SEQ ID NO: 25), allele A of PE-0056549 (SEQ ID NO: 29), and allele T of PE-0056550 (SEQ ID NO: 33);
allele combination “D” comprising allele A of PE-0014628 (SEQ ID NO: 1), allele C of PE-0056542 (SEQ ID NO: 5), allele A of PE-0056543 (SEQ ID NO: 9), allele T of PE-0056544 (SEQ ID NO: 13), allele C of PE-0056545 (SEQ ID NO: 17), allele C of PE-0056546 (SEQ ID NO: 21), allele C of PE-0056547 (SEQ ID NO: 25), allele A of PE-0056549 (SEQ ID NO: 29), allele T of PE-0056550 (SEQ ID NO: 33), and allele C of PE-0021476 (SEQ ID NO: 37); and
allele combination “E” comprising allele A of PE-0056549 (SEQ ID NO: 29) and allele T of PE-0056550 (SEQ ID NO:33).

21. The plant according to claim 17, wherein the QTL is homozygously present in the genome of the plant.

22. The plant according to claim 17, wherein the QTL is heterozygously present in the genome of the plant.

23. The plant according to claim 17, wherein the QTL co-segregates with the gene CA06g26150 and/or the gene CA06g26250.

24. The plant according to claim 23, wherein the resistance is caused by at least one loss-of-function mutation in the sequence of the gene CA06g26150 or in the sequence of the gene CA06g26250.

25. The plant according to claim 17, wherein the plant is a progeny of, or is derived from a Capsicum annuum plant of ST17-1157, representative seeds of which have been deposited under NCIMB accession number NCIMB 43602.

26. The plant according to claim 17, which is a Capsicum annuum, Capsicum baccatum, Capsicum frutescens, Capsicum chinense, Capsicum pubescens or Capsicum chacoense plant

27. The plant according to claim 26, which is a Capsicum annuum plant.

28. The plant according to claim 17, which is a bell pepper.

29. A Capsicum seed, which can be grown into a Capsicum plant according to claim 17.

30. A method for producing a Capsicum plant according to claim 17, or a plant part, cell or seed thereof, comprising:
(i) using a Capsicum plant resistant to powdery mildew caused by the fungal pathogen Leveillula taurica, comprising introgressed in its genome a quantitative trait locus (QTL) conferring resistance to powdery mildew, wherein the QTL is located on chromosome 6 within the genomic interval delimited by the marker PE-0014628 (SEQ ID NO: 1) and the marker PE-0021476 (SEQ ID NO: 37), as a breeding partner in a breeding program, thereby transferring the QTL to a progeny Capsicum plant, wherein the progeny Capsicum plant is resistant to powdery mildew caused by the fungal pathogen Leveillula taurica, or
(ii) introducing by mutagenesis or genome editing one or more mutations in the genome of Capsicum plants, at a locus within the genomic interval delimited by the marker PE-0014628 (SEQ ID NO: 1) and the marker PE-0021476 (SEQ ID NO: 37) and selecting a mutant plant, wherein the plant is resistant to powdery mildew caused by the fungal pathogen Leveillula taurica.

31. The method of claim 30, wherein step (i) comprises:
(i) crossing the Capsicum plant resistant to powdery mildew and a susceptible or less resistant Capsicum plant, thus generating a F1 population,
(ii) selfing the F1 population to create a F2 population, and
(iii) selecting resistant individuals in the progeny thus obtained.

32. The method of claim 31, further comprising self-pollinating and/or backcrossing one or several times the resistant plant obtained at step c) and selecting a resistant plant in the progeny thus obtained,

33. A method for detecting and/or selecting Capsicum plants comprising in their genome a QTL on chromosome 6, the QTL conferring resistance to powdery mildew, comprising detecting a molecular marker linked to the QTL within the chromosomal region delimited by marker PE-0014628 (SEQ ID NO: 1) and marker PE-0021476 (SEQ ID NO: 37).

34. The method of claim 33, comprising the detection of at least one of the following alleles: A of PE-0014628 (SEQ ID NO: 1), allele C of PE-0056542 (SEQ ID NO: 5), allele A of PE-0056543 (SEQ ID NO: 9), allele T of PE-0056544 (SEQ ID NO: 13), allele C of PE-0056545 (SEQ ID NO: 17), allele C of PE-0056546 (SEQ ID NO: 21), allele C of PE-0056547 (SEQ ID NO: 25), allele A of PE-0056549 (SEQ ID NO: 29), allele T of PE-0056550 (SEQ ID NO: 33), and allele C of PE-0021476 (SEQ ID NO: 37).

35. The method of claim 34, comprising the detection of at least one of the following alleles: allele A of PE-0056543 (SEQ ID NO: 9), allele T of PE-0056544 (SEQ ID NO: 13), allele C of PE-0056545 (SEQ ID NO: 17), and allele C of PE-0056546 (SEQ ID NO: 21).

36. The method of claim 34, comprising detecting one of the following allele combinations:
allele combination “A” comprising allele A of PE-0056543 (SEQ ID NO: 9), allele T of PE-0056544 (SEQ ID NO: 13), allele C of PE-0056545 (SEQ ID NO: 17), and allele C of PE-0056546 (SEQ ID NO: 21);
allele combination “B” comprising allele C of PE-0056542 (SEQ ID NO: 5), allele A of PE-0056543 (SEQ ID NO: 9), allele T of PE-0056544 (SEQ ID NO: 13), allele C of PE-0056545 (SEQ ID NO: 17), allele C of PE-0056546 (SEQ ID NO: 21) and allele C of PE-0056547 (SEQ ID NO: 25);
allele combination “C” comprising allele C of PE-0056542 (SEQ ID NO: 5), allele A of PE-0056543 (SEQ ID NO: 9), allele T of PE-0056544 (SEQ ID NO: 13), allele C of PE-0056545 (SEQ ID NO: 17), allele C of PE-0056546 (SEQ ID NO: 21), allele C of PE-0056547 (SEQ ID NO: 25), allele A of PE-0056549 (SEQ ID NO: 29), and allele T of PE-0056550 (SEQ ID NO: 33);
allele combination “D” comprising allele A of PE-0014628 (SEQ ID NO: 1), allele C of PE-0056542 (SEQ ID NO: 5), allele A of PE-0056543 (SEQ ID NO: 9), allele T of PE-0056544 (SEQ ID NO: 13), allele C of PE-0056545 (SEQ ID NO: 17), allele C of PE-0056546 (SEQ ID NO: 21), allele C of PE-0056547 (SEQ ID NO: 25), allele A of PE-0056549 (SEQ ID NO: 29), allele T of PE-0056550 (SEQ ID NO: 33), and allele C of PE-0021476 (SEQ ID NO: 37); and
allele combination “E” comprising allele A of PE-0056549 (SEQ ID NO: 29) and allele T of PE-0056550 (SEQ ID NO:33).
